(19) United States
(12) Patent Application Publication
(10) Pub. No.: US 2021/0115517 A1
(43) Pub. Date: Apr. 22, 2021

(54) METHODS OF TREATING PROSTATE CANCER BASED ON MOLECULAR SUBTYPES

(71) Applicant: Janssen Pharmaceutica NV

(72) Inventors: Clemente Aguilar, Spring House, PA (US); Michael Gormley, Spring House, PA (US); Shibu Thomas, Spring House, PA (US)

(21) Appl. No.: 16/776,734

(22) Filed: Jan. 30, 2020

Related U.S. Application Data
(60) Provisional application No. 62/938,318, filed on Nov. 20, 2019.
(60) Provisional application No. 62/825,001, filed on Mar. 27, 2019.
(60) Provisional application No. 62/824,968, filed on Mar. 27, 2019.
(60) Provisional application No. 62/801,610, filed on Feb. 5, 2019.
(60) Provisional application No. 62/801,609, filed on Feb. 5, 2019.
(60) Provisional application No. 62/799,037, filed on Jan. 30, 2019.
(60) Provisional application No. 62/799,036, filed on Jan. 30, 2019.

(51) Int. Cl. C12Q 1/6886; A61K 31/4439; A61K 45/06

(52) U.S. Cl. CPC C12Q 1/6886; A61K 31/4439; C12Q 2600/158; C12Q 2600/106; A61K 45/06

(57) ABSTRACT

Provided are methods of treating prostate cancer in a human male comprising administration of apalutamide and androgen deprivation therapy to a human male having prostate cancer (e.g., nmCRPC) if a biological sample obtained from the human male is determined to have a specific molecular subtype of prostate cancer, a specific classifier score, or increased or decreased expression of a signature class. The molecular subtypes include luminal-like or basal-like molecular subtype. Also provided are methods of using molecular signatures and genomic classifier scores, such as four co-regulated signature classes, metastasis risk based on a genomic classifier score, or a combination thereof, as prognostic indicators of apalutamide and androgen deprivation therapy in human males having prostate cancer, for improved treatment benefit.

### CROSS REFERENCE TO RELATED APPLICATIONS

This application claims the benefit of U.S. Provisional Application No. 62/799,036, filed on Jan. 30, 2019, U.S. Provisional Application No. 62/799,037, filed on Jan. 30, 2019, U.S. Provisional Application No. 62/801,609, filed on Feb. 5, 2019, U.S. Provisional Application No. 62/801,610, filed on Feb. 5, 2019, U.S. Provisional Application No. 62/824,968, filed on Mar. 27, 2019, U.S. Provisional Application No. 62/825,001, filed on Mar. 27, 2019, and U.S. Provisional Application No. 62/938,318, filed on Nov. 20, 2019. The entire contents of the above applications are incorporated herein by reference.

### BACKGROUND

Prostate cancer is the second most frequently diagnosed cancer and the sixth leading cause of cancer death in males worldwide. Prostate cancer rates are higher in developed countries than in the rest of the world, where many of the risk factors for prostate cancer are more common, including longer life expectancy and diets high in red meat. Also, there is a higher detection rate in developed countries where there is more access to screening programs. In patients who undergo treatment, the most important clinical prognostic indicators of disease outcome are the stage, pretherapy PSA level, and Gleason score. In general, the higher the grade and the stage, the poorer the prognosis. While treatment can be curative at early stages, with treatment in later stages of prostate cancer, however, biochemical recurrence in some patients will occur. Androgen deprivation therapy (ADT) is the main treatment for prostate cancer, and although ADT is initially effective, disease progression to castration-resistance prostate cancer (CRPC) eventually occurs in almost all patients. There is a need for improved methods of treating prostate cancer.

### SUMMARY

In some embodiments, the invention relates to molecular signatures as prognostic indicators of an androgen-receptor inhibitor (e.g., apalutamide (APA) and an androgen deprivation therapy (ADT) (APA+ADT)) in human males having prostate cancer (e.g., non-metastatic castration resistant prostate cancer (nmCRPC)).

In one aspect, the present invention provides methods of providing improved treatment benefit of prostate cancer (e.g., nmCRPC) in a human male using an androgen-receptor inhibitor (e.g., APA) and an androgen deprivation therapy (ADT) (e.g., APA+ADT), comprising, consisting of and/or consisting essentially of:

administering a therapeutically effective amount of the androgen-receptor inhibitor (e.g., APA) and a therapeutically effective amount of the ADT to the human male if a biological sample obtained from the human male is determined to have:
a) a luminal-like or a basal-like molecular subtype of prostate cancer;
b) a genomic classifier score of greater than about 0.6;
c) an increased expression of at least one signature of Class One co-regulated signatures;
d) an increased expression of at least one signature of Class Two co-regulated signatures;
e) a decreased expression of at least one signature of Class Three co-regulated signatures;
f) an increased expression of at least one signature of Class Four co-regulated signatures; or a combination thereof.

In another aspect, the present invention provides methods of treating prostate cancer (e.g., nmCRPC) in a human male, said method comprising, consisting of and/or consisting essentially of:

administering a therapeutically effective amount of an androgen-receptor inhibitor (e.g., APA) and a therapeutically effective amount of an androgen deprivation therapy (ADT) to the human male if a biological sample obtained from the human male is determined to have:
a) a luminal-like or a basal-like molecular subtype of prostate cancer;
b) a genomic classifier score of greater than about 0.6;
c) an increased expression of at least one signature of Class One co-regulated signatures;
d) an increased expression of at least one signature of Class Two co-regulated signatures;
e) a decreased expression of at least one signature of Class Three co-regulated signatures;
f) an increased expression of at least one signature of Class Four co-regulated signatures; or a combination thereof.

In another aspect, the present invention provides methods of predicting a human male having prostate cancer (e.g., nmCRPC) to have an improved benefit from administration of a therapeutically effective amount of an androgen-receptor inhibitor (e.g., APA) and a therapeutically effective amount of an androgen deprivation therapy (ADT) (e.g., APA+ADT) relative to sole administration of a therapeutically effective amount of the ADT, said method comprising, consisting of and/or consisting essentially of:
a) determining if a biological sample obtained from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof, and
b) predicting that the human male to have an improved benefit from administration of the therapeutically effective amount of the androgen-receptor inhibitor (e.g., APA) and the therapeutically effective amount of the ADT relative to sole administration of the therapeutically effective amount of the ADT based on:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof.

In another aspect, the present invention provides methods of improving response to treating non-metastatic castration resistant prostate cancer (nmCRPC) in a human male using a combined administration of a therapeutically effective amount of an androgen-receptor inhibitor (e.g., APA) and a therapeutically effective amount of an androgen deprivation therapy (ADT) (e.g., APA+ADT) relative to sole administration of a therapeutically effective amount of the ADT, the method comprising, consisting of and/or consisting essentially of:
a) determining if a biological sample obtained from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof, and
b) improving response to combined administration of the therapeutically effective amount of the androgen-receptor inhibitor (e.g., APA) and the therapeutically effective amount of the ADT relative to sole administration of the therapeutically effective amount of the ADT, based on:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof.

In another aspect, the present invention provides methods of identifying a human male diagnosed with prostate cancer (e.g., nmCRPC) predicted to have an improved treatment benefit from a therapeutically effective amount of an androgen-receptor inhibitor (e.g., APA) and a therapeutically effective amount of an androgen deprivation therapy (ADT) (e.g., APA+ADT) relative to sole administration of a therapeutically effective amount of the ADT, comprising, consisting of and/or consisting essentially of:
a) determining if a biological sample obtained from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof, and
b) predicting that the human male to have an improved benefit from administration of the therapeutically effective amount of the androgen-receptor inhibitor (e.g., APA) and the therapeutically effective amount of the ADT relative to sole administration of the therapeutically effective amount of the ADT based on:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof.

In yet another aspect, the present invention provides methods of predicting an improvement of treatment response of prostate cancer (e.g., nmCRPC) to a therapeutically effective amount of an androgen-receptor inhibitor (e.g., APA) and a therapeutically effective amount of an androgen deprivation therapy (ADT) (e.g., APA+ADT) relative to sole administration of a therapeutically effective amount of the ADT in a human male, comprising, consisting of and/or consisting essentially of:
a) determining if a biological sample from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures,
   or a combination thereof, and
b) predicting an improvement of response to the therapeutically effective amount of the androgen-receptor inhibitor (e.g., APA) and the therapeutically effective amount of the ADT relative to sole administration of the therapeutically effective amount of the ADT, based on:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures,
   or a combination thereof.

In some embodiments, metastasis-free survival (MFS) of combined administration of APA+ADT is improved by at least about 6 months relative to sole administration of ADT alone.

In some embodiments, second progression-free survival (PFS2) of combined administration of APA+ADT is improved by at least about 6 months relative to sole administration of ADT alone (i.e., sole administration of ADT).

In some embodiments, the method further comprises obtaining the biological sample from the human male.

In some embodiments, the biological sample is determined to have a luminal-like molecular subtype of prostate cancer.

In some embodiments, the human male is determined to have a high risk of metastasis based on the genomic classifier score of greater than about 0.6. In some embodiments, the human male is determined to have a high risk of metastasis based on the genomic classifier score of greater than 0.6.

In some embodiments, the biological sample is determined to have an increased expression of at least one signature of the Class One co-regulated signatures.

In some embodiments, the biological sample is determined to have an increased expression of at least one signature of the Class Two co-regulated signatures.

In some embodiments, the biological sample is determined to have a decreased expression of at least one signature of the Class Three co-regulated signatures.

In some embodiments, the biological sample is determined to have an increased expression of at least one signature of the Class Four co-regulated signatures.

In some embodiments, the prostate cancer is nmCRPC.

In some embodiments of the invention, the metastasis-free survival is improved relative to administration of ADT alone. In some embodiments of the invention, second progression-free survival is improved relative to administration of ADT alone.

In some embodiments, the human male has undergone a prostatectomy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1B compare luminal-like and basal-like subtypes of prostate cancer. FIG. 1A (modified from Smith et al., PNAS 112(47): E6544-52 (2013), FIG. 4A) shows that the basal-like subtype of prostate cancer is enriched in metastasis compared to local disease. FIG. 1B (adapted from Zhang et al., Nat Commun. 7:10718 (2016), FIG. 1G) compares the functional differences between luminal-like and basal-like subtypes in the prostate.

FIG. 2 (adapted from Zhao et al., JAMA Oncol., 3(12):1663-72 (2017)) depicts the frequencies of molecular subtypes of prostate tumors as reported by Zhao et al., JAMA Oncol., 3(12):1663-72 (2017) (hereinafter “Zhao et al.” or “PAM50”) and Zhang et al. Nature Communications 7: 10798 (2016) (hereinafter “Zhang et al.”). Both references are incorporated herein in their entirety.

FIG. 3 shows that the basal-like subtype of prostate cancer is enriched in patients in the SPARTAN trial. The top panel of FIG. 3 is based on Zhao et al., JAMA Oncol., 3(12):1663-72 (2017); and the bottom panel of FIG. 3 is based on Zhang et al., Nat Commun. 7:10718 (2016) and Smith et al., PNAS 112(47): E6544-52 (2013).

FIG. 4 illustrates that basal-like tumors have a worse prognosis compared to luminal-like tumors in the SPARTAN trial patients.

FIG. 5 depicts the SPARTAN study design and sample collection and analysis.

FIG. 6 depicts a heat map for differentially expressed genes in the SPARTAN biomarker population.

FIGS. 7A and 7B depict metastasis-free survival (MFS) by treatment arm in patients with luminal-like (FIG. 7A) and basal-like (FIG. 7B) subtypes. Both luminal-like tumors and basal-like tumors show an improved benefit to apalutamide (APA) and androgen deprivation therapy (ADT) (APA+ADT) compared to ADT alone (PBO+ADT) in the SPARTAN trial patients.

FIGS. 8A and 8B depict MFS by basal-like and luminal-like subtypes in the ADT alone (PBO+ADT) (FIG. 8A) and APA+ADT (FIG. 8B) treatment arms of SPARTAN. Luminal-like tumors show a maximal benefit in MFS to APA+ADT compared to ADT alone (PBO+ADT) in the SPARTAN trial patients.

FIGS. 9A-9B depict results on luminal-like and basal-like tumors. FIGS. 9A and 9B depict second progression-free survival (PFS2) by treatment arm in patients with luminal-like (FIG. 9A) and basal-like (FIG. 9B) subtypes. Both luminal-like tumors and basal-like tumors show an improved benefit to apalutamide (APA) and androgen deprivation therapy (ADT) (APA+ADT) compared to ADT alone in the SPARTAN trial patients. FIGS. 9C and 9D depict PFS2 with luminal-like and basal-like subtypes in the ADT (FIG. 9C) and APA+ADT (FIG. 9D) treatment arms of SPARTAN.

FIG. 10 depicts the biological pathways associated with the basal-like molecular subtype.

FIG. 11 shows that DECIPHER® GCs are associated with metastasis. The top panel is based on Karnes et al., J Urol. 190(6): 2047-53 (2013), FIG. 3.

FIGS. 12A and 12B depict MFS by DECIPHER® GC score in the ADT alone (PBO+ADT) (FIG. 12A) and APA+ADT (FIG. 12B) treatment arms of SPARTAN. FIG. 12A shows that DECIPHER® GC high risk patients are associated with poor prognosis when treated with ADT in the SPARTAN cohort. FIG. 12B shows that DECIPHER® GC high and low-to-average risk patients have similar metastasis-free survival (WS) when treated with APA+ADT in the SPARTAN cohort.

FIGS. 13A and 13B depict MFS by treatment arm in patients with high (FIG. 13A) and low-to-average (FIG. 13B) DECIPHER® GC score. DECIPHER® GC high risk patients show maximal benefit in MFS when treated with APA+ADT compared to ADT in the SPARTAN cohort.

FIGS. 14A-14K depict the methods of Example 2. FIG. 14A depicts the overall method steps. FIG. 14B depicts the hierarchical clustering heatmap. Each row represents a signature, and each column represents a patient sample. FIGS. 14C and 14D are boxplots of raw data and ranked data, respectively. FIG. 14E depicts quantile normalized data of the 160 signatures. Value ranges from 1 to 233. FIG. 14F depicts the selection of the cluster number (k=4) based on the relative change in the area under the empirical cumulative distribution. FIGS. 14G-14J depict pairwise Pearson correlation between matrices. Diagonal indicates x and y axis labels (e.g., signature 2 is 75% correlated with signature 3 in FIG. 14I). Top right: correlation coefficient. Bottom left: scatterplot of correlation between the two signatures. FIG. 14K depicts the signature expression patterns of the 233 SPARTAN samples. The tumor samples were divided into three subtypes (1: High Basal/NE Like, 51.7%; 2: High-Risk and Steroid Homeogenesis, 33.9%; and 3: High Immune, 15.2%). The 160 signatures were divided into four Classes (Class One: 24.38%; Class Two: 31.87%, Class Three: 25%, and Class Four: 18.75%).

FIGS. 15A-15E depict results on genomic_gleason_grade_2, a representative Class One signature. FIGS. 15A and 15B depict Metastasis-free survival (MFS) by expression of genomic_gleason_grade_2 in the ADT (FIG. 15A) and APA+ADT (FIG. 15B) treatment arms of SPARTAN. FIGS. 15C and 15D depict MFS by treatment arm in patients with high (FIG. 15C) and low (FIG. 15D) expression of genomic_gleason_grade_2. FIG. 15E depicts association of expression of genomic_gleason_grade_2 with relative risk by treatment arm.

FIGS. 16A-16E depict results on hallmark_cholesterol_homeostasis, a representative Class Two signature. FIGS. 16A and 16B depict MFS by expression of hallmark_cholesterol_homeostasis in the ADT (FIG. 16A) and APA+ADT (FIG. 16B) treatment arms of SPARTAN. FIGS. 16C and 16D depict MFS by treatment arm in patients with high (FIG. 16C) and low (FIG. 16D) expression of hallmark_cholesterol_homeostasis. FIG. 16E depicts association of expression of hallmark_cholesterol_homeostasis with relative risk by treatment arm.

FIGS. 17A-17E depict results on beltran2016_1, a representative Class Three signature. FIGS. 17A and 17B depict MFS by expression of beltran2016_1 in the ADT (FIG. 17A) and APA+ADT (FIG. 17B) treatment arms of SPARTAN. FIGS. 17C and 17D depict MFS by treatment arm in patients with high (FIG. 17C) and low (FIG. 17D) expression of beltran2016_1. FIG. 17E depicts association of expression of beltran2016_1 with relative risk by treatment arm.

FIGS. 18A-18E depict results on hallmark_IL2_JAK_STAT5_signaling, a representative Class Four signature. FIGS. 18A and 18B depict MFS by expression of hallmark_IL2_JAK_STAT5_signaling in the ADT (FIG. 18A) and APA+ADT (FIG. 18B) treatment arms of SPARTAN. FIGS. 18C and 18D depict MFS by treatment arm in patients with high (FIG. 18C) and low (FIG. 18D) expression of hallmark_IL2_JAK_STAT5_signaling. FIG. 18E depicts association of expression of hallmark_IL2_JAK_STAT5_signaling with relative risk by treatment arm.

### DETAILED DESCRIPTION

A description of example embodiments follows.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the invention, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

The terminology used herein is for the purpose of describing particular embodiments only and is not intended to be limiting. As used herein, the articles “a,” “an” and “the” should be understood to include plural reference unless the context clearly indicates otherwise.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

The terms “human male” and “patient” can be used interchangeably herein. A “human male” includes a male human whose prostate cancer is being treated.

The term “cancer” as used herein refers to an abnormal growth of cells which tend to proliferate in an uncontrolled way and, in some cases, to metastasize (spread).

The term “prostate cancer” as used herein refers to histologically or cytologically confirmed adenocarcinoma of the prostate.

The term “locally advanced prostate cancer” refers to prostate cancer where all actively cancerous cells appear to be confined to the prostate and the associated organs or neighbor organs (e.g., seminal vesicle, bladder neck, and rectal wall).

The term “high-risk localized prostate cancer” refers to locally advanced prostate cancer that has a probability of developing metastases or recurrent disease after primary therapy with curative intent.

The term “castration-sensitive prostate cancer” refers to cancer that is responsive to androgen-deprivation therapy (ADT) either as localized disease or biochemical relapse.

The terms “non-metastatic castration-sensitive prostate cancer” “nmCRPC,” or “NM-CRPC,” as used interchangeably herein refer to prostate cancer that has not spread (metastasized) in a male, and that is responsive to androgen-deprivation therapy (ADT). In some embodiments, non-metastatic castration-sensitive prostate cancer is assessed with bone scan and computed tomography (CT) or magnetic resonance imaging (MRI) scans.

Patients with nmCRPC can have rising prostate-specific antigen and castrate testosterone levels, with no radiological findings of metastatic disease on computed tomography and bone scan.

The term “CRPC” as used herein refers to castration-resistant prostate cancer. CRPC is prostate cancer that continues to grow despite the suppression of male hormones that fuel the growth of prostate cancer cells.

The term “chemotherapy naive metastatic castration-resistant prostate cancer” refers to metastatic castration-resistant prostate cancer that has not been previously treated with a chemotherapeutic agent.

The terms “luminal-like” and “luminal” are used interchangeably herein.

The terms “basal-like” and “basal” are used interchangeably herein.

The term “high risk nmCRPC” refers to probability of a man with nmCRPC developing metastases.

As used herein, the terms “Class One co-regulated signatures,” “Class One signatures,” “signatures related to prognosis,” “prognosis related signatures,” “risk signatures,” and “high-risk signatures” are interchangeable, and comprise the signatures provided in Table 4. These signatures were found to predict higher risk for metastasis.

As used herein, the terms “Class Two co-regulated signatures,” “Class Two signatures,” “signatures related to steroid homeostasis,” “steroid homeostasis related signatures,” and “steroid homeostasis signatures” are interchangeable, and comprise the signatures provided in Table 5. These signatures were found to be related to steroid homeostasis.

As used herein, the terms “Class Three co-regulated signatures,” “Class Three signatures,” “Neuroendocrine signature,” “NE signatures” “Neuroendocrine-Basal signatures,” “Adeno with NE like features,” and “hormonal therapy non-responsive basal and neuroendocrine like signatures” are interchangeable, and comprise the signatures provided in Table 6. These signatures were found to be associated to prostate cancers resistant to androgen receptor (AR) directed therapy (Beltran et al, Divergent clonal evolution of castration-resistant neuroendocrine prostate cancer, Nat Med. 2016; 22(3)298-305).

As used herein, the terms “Class Four co-regulated signatures,” “Class Four signatures,” “Hallmark gene sets,” “stromal/immune signatures,” “immune/stromal signatures,” and “immune and stromal IL2/ IL-6-JAK-STATS like signatures” are interchangeable, and comprise the signatures provided in Table 7.

The term “metastasis-free survival” or “MFS” refers to the percentage of human males in a study who have survived without cancer spread for a defined period of time or death.

S is usually reported as time from the beginning of enrollment, randomization or treatment in the study. MFS is reported for an individual or a study population. In the context of treatment of CRPC with an androgen-receptor inhibitor, an increase in the metastasis-free survival is the additional time that is observed without cancer having spread or death, whichever occurs first, as compared to treatment with placebo. Specifically, it is the time from randomization to the first detection of distant metastasis on imaging or death.

The term “time to metastasis” is the time from randomization to the time of the scan that shows first evidence of BICR-confirmed radiographically detectable bone or soft tissue distant metastasis.

The phrases “second progression-free survival”, “progression-free survival with the first subsequent therapy,” or “PFS2,” used interchangeably herein, are defined as the time from randomization to investigator-assessed disease progression (PSA, radiographic, symptomatic, or any combination) during first subsequent anti-cancer therapy or death (any cause) prior to the start of the second subsequent anti-cancer therapy, whichever occurs first. Progression data for human males without documented progression after subsequent therapy is censored at the last date known to be progression-free or date of death. In some embodiments, administration of a safe and effective amount of an androgen-receptor inhibitor provides improved anti-tumor activity as measured progression-free survival with the first subsequent therapy.

The term “progression-free survival with the first subsequent therapy (PFS2)” is defined as the time from randomization to investigator-assessed disease progression (PSA, radiographic, symptomatic, or any combination) during first subsequent anti-cancer therapy or death (any cause) prior to the start of the second subsequent anti-cancer therapy, whichever occurs first.

Progression data for human males without documented progression after subsequent therapy is censored at the last date known to be progression-free or date of death. In some embodiments, administration of a safe and effective amount of an androgen-receptor inhibitor provides improved anti-tumor activity as measured by progression-free survival with the first subsequent therapy.

Prostate specific antigen response and time to PSA progression is assessed at the time of the primary analysis of WS according to the Prostate Cancer Working Group (PCWG2) criteria. (H. I. Scher, M. J. Morris, E. Basch, G. Heller, 2011, J. Clin Oncol.) The time to PSA progression is calculated as the time from randomization to the time when the criteria for PSA progression according to PCWG2 are met.

The term “progression-free survival” is based on RECIST v1.1 and is defined in LH Schwartz, 2016, Euro J of Cancer 2016, incorporated herein by reference.

For human males with at least one measurable lesion, progressive disease is defined as at least a 20% increase in the sum of diameters of target lesions taking as reference the smallest sum on study (this includes the baseline sum if that is the smallest on study). In addition to the relative increase of 20%, the sum must also demonstrate an absolute increase of at least 5 mm. Furthermore, the appearance of one or more new lesions is also considered progression. For human males with only non-measurable disease observed on CT or MRI scans, unequivocal progression (representative of overall disease status change) or the appearance of one or more new lesions was considered progression. For new bone lesions detected on bone scans, a second imaging modality (e.g., CT or MRI) was required to confirm progression. In some embodiments, administration of a safe and effective amount of an androgen-receptor inhibitor provides improved anti-tumor activity as measured by progression-free survival rate.

The term “time to symptomatic progression” is defined as the time from randomization to documentation in the CRF of any of the following (whichever occurs earlier): (1) development of a skeletal-related event (SRE): pathologic fracture, spinal cord compression, or need for surgical intervention or radiation therapy to the bone; (2) pain progression or worsening of disease related symptoms requiring initiation of a new systemic anti-cancer therapy; or (3) development of clinically significant symptoms due to loco-regional tumor progression requiring surgical intervention or radiation therapy. In some embodiments, administration of a safe and effective amount of an androgen-receptor inhibitor provides improved anti-tumor activity as measured by time to symptomatic progression.

The term “overall survival” is defined as the time from randomization to the date of death due to any cause. Survival data for human males who are alive at the time of the analysis was to be censored on the last known date that they were alive. In addition, for human males with no postbaseline information survival, data was to be censored on the date of randomization; for human males who are lost to follow-up or who withdraw consent, data is censored on the last known date that they were alive. In some embodiments, administration of a safe and effective amount of an antiandrogen provides improved anti-tumor activity as measured by overall survival.

The term “time to initiation of cytotoxic chemotherapy” is defined as the time from randomization to documentation of a new cytotoxic chemotherapy being administered to the human male (e.g., survival follow-up CRF). Time to initiation of cytotoxic chemotherapy for human males who do not start a cytotoxic chemotherapy is censored on the date of last contact. In some embodiments, administration of a safe and effective amount of an androgen-receptor inhibitor provides improved anti-tumor activity as measured by time to cytotoxic chemotherapy.

The term “survival benefit” as used herein means an increase in survival of the patient from time of randomization on the trial of administered drug to death. In some embodiments, the survival benefit is about 1, about 2, about 3, about 4, about 5, about 6, about 7, about 8, about 9, about 10, about 15, about 20, about 25, about 30, about 35, about 40, about 45, about 50, about 55, about 60, about 80, about 100 months or greater than 100 months.

The term “delay in symptoms related to disease progression” as used herein means an increase in time in the development of symptoms such as pain, urinary obstruction and quality of life considerations from the time of randomization on the trial of administered drug.

The term “randomization” as it refers to a clinical trial refers to the time when the patient is confirmed eligible for the clinical trial and gets assigned to a treatment arm.

Androgen-Receptor Inhibitors

As used herein, the term “androgen-receptor inhibitor” refers to active pharmaceutical ingredients that are capable of preventing or inhibiting the biologic effects of androgens on normally responsive tissues in the body.

As used herein, the term “AR antagonist” or “AR inhibitor” are used interchangeably herein and refer to an agent that inhibits or reduces at least one activity of an AR polypeptide. Example AR activities include, but are not limited to, co-activator binding, DNA binding, ligand binding, or nuclear translocation.

As used herein, a “full antagonist” refers to an antagonist which, at an effective concentration, essentially completely inhibits an activity of an AR polypeptide. “Essentially completely” means at least about 80%, at least about 90%, at least about 95%, at least about 96%, at least about 97%, at least about 98%, at least about 99%, or greater inhibition of the activity of an AR polypeptide.

As used herein, a “partial antagonist” refers an antagonist that is capable of partially inhibiting an activity of an AR polypeptide, but that, even at a highest concentration is not a full antagonist.

Example androgen-receptor inhibitors include, but are not limited to, flutamide, nilutamide, bicalutamide, 4-[7-(6-cyano-5-trifluoromethylpyridin-3-yl)-8-oxo-6-thioxo-5,7-diazaspiro[3.4]oct-5-yl]-2-fluoro-N-methylbenzamide (also known as apalutamide or ARN-509), 4-(3-(4-cyano-3-(trifluoromethyl)phenyl)-5,5-dimethyl-4-oxo-2-thioxoimidazolidin-1-yl)-2-fluoro-N-methylbenzamide (also known as MDV3100 or enzalutamide), and darolutamide.

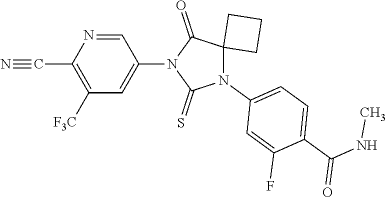 [SMILES: CNC(=O)c1ccc(N2C(=S)N(c3cnc(C#N)c(C(F)(F)F)c3)C(=O)C23CCC3)cc1F]

4-[7-(6-cyano-5-trifluoromethylpyridin-3-yl)-8-oxo-6-thioxo-5,7-diazaspiro[3.4]oct-5-yl]-2-fluoro-N-methylbenzamide (apalutamide).

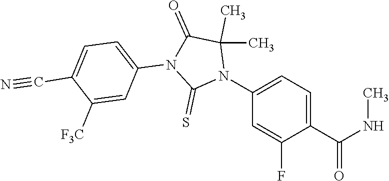 [SMILES: CNC(=O)c1ccc(N2C(=S)N(c3ccc(C#N)c(C(F)(F)F)c3)C(=O)C2(C)C)cc1F]

4-(3-(4-cyano-3-(trifluoromethyl)phenyl)-5,5-dimethyl-4-oxo-2-thioxoimidazolidin-1-yl)-2-fluoro-N-methylbenzamide (enzalutamide).

In some embodiments, an androgen-receptor inhibitor binds to an AR polypeptide at or near the ligand binding site of the AR polypeptide.

In some embodiments, an androgen-receptor inhibitor contemplated in the methods described herein inhibits AR nuclear translocation, such as darolutamide, DNA binding to androgen response elements, and coactivator recruitment. In some embodiments, an androgen-receptor inhibitor contemplated in the methods described herein exhibits no agonist activity in AR-overexpressing prostate cancer cells.

Apalutamide is a second next-generation androgen-receptor inhibitor that binds directly to the ligand binding domain of AR, impairing nuclear translocation, AR binding to DNA and AR target gene modulation, thereby inhibiting tumor growth and promoting apoptosis. Apalutamide binds AR with greater affinity than bicalutamide, and induces partial or complete tumor regression in noncastrate hormone-sensitive and bicalutamide -resistant human prostate cancer xenograft models (Clegg et al. Cancer Res. Mar. 15, 2012 72; 1494). Apalutamide lacks the partial agonist activity seen with bicalutamide in the context of AR overexpression. Apalutamide is the active ingredient of ERLEADA®. Additional information regarding apalutamide can be found, for example, in the prescribing information product insert for ERLEADA® (apalutamide) tablets, http://www_janssenlabels.com/package-insert/product-monograph/prescribing-information/ERLEADA-pi_pdf, which is incorporated herein by reference.

Darolutamide, BAY1841788 or ODM-201, is an AR antagonist that includes two diastereomers—ORM-16497 and ORM-16555. It has activity against known AR mutants that confer resistance to other second-generation antiandrogens. Darolutamide binds to the AR with high affinity, and impairs subsequent androgen-induced nuclear translocation of AR and transcription of AR gene target. Matsubara, N., Mukai, H., Hosono, A. et al., Cancer Chemother Pharmacol 80: 1063 (2017).

Castration-resistant prostate cancer is categorized as non-metastatic or metastatic, depending on whether or not the prostate cancer has metastasized to other parts of the body.

The term “androgen-deprivation therapy (ADT)” refers to the reduction of androgen levels in a prostate cancer patient to castrated levels of testosterone (<50 ng/dL). Such treatments can include orchiectomy or the use of gonadotropin-releasing hormone agonists or antagonists. ADT includes surgical castration (orchiectomy) and/or the administration of luteinizing hormone-releasing hormone (“LHRH”) agonists to a human. Examples of LHRH agonists include goserelin acetate, histrelin acetate, leuprolide acetate, and triptorelin palmoate.

The terms “co-administration” or the like, as used herein, encompass administration of the selected therapeutic agents to a single patient, and are intended to include treatment regimens in which the agents are administered by the same or different route of administration and/or at the same or different time.

The term “pharmaceutical combination” as used herein, means a product that results from the mixing or combining of more than one active ingredient and includes both fixed and non-fixed combinations of the active ingredients.

The term “FDHT-PET” refers to 18F-16P-fluoro-5a-dihydrotestosterone Positron Emission Tomography and is a technique that uses a tracer based on dihydrotestosterone, and allows for a visual assessment of ligand binding to the androgen receptor in a patient. It may be used to evaluate pharmacodynamics of an androgen receptor directed therapy.

The term “continuous daily dosing schedule” refers to the administration of a particular therapeutic agent without any drug holidays from the particular therapeutic agent. In some embodiments, a continuous daily dosing schedule of a particular therapeutic agent comprises administration of a particular therapeutic agent every day at roughly the same time each day.

The terms “treat” and “treatment” refer to the treatment of a cancer in a human afflicted with a pathological condition and refers to an effect that alleviates the condition by killing the cancerous cells, but also to an effect that results in the inhibition of the progress of the condition, and includes a reduction in the rate of progress, a halt in the rate of progress, amelioration of the condition, and cure of the condition. Treatment as a prophylactic measure (i.e., prophylaxis) is also included.

The term, “drug product” or “approved drug product” is product that contains an active pharmaceutical ingredient that has been approved for marketing for at least one indication by a governmental authority, e.g., the Food and Drug Administration or the similar authority in other countries.

One aspect of the invention relates to a method of providing improved treatment benefit to prostate cancer (e.g., nmCRPC) in a human male with an approved drug product that contains an androgen-receptor inhibitor (e.g., apalutamide (APA)) and an approved drug product that contains an androgen deprivation therapy (ADT) (e.g., APA+ADT), in separate or the same dosage form, comprising, consisting of and/or consisting essentially of:

administering a therapeutically effective amount of the androgen-receptor inhibitor and a therapeutically effective amount of the ADT to the human male if a biological sample obtained from the human male is determined to have:
a) a luminal-like or a basal-like molecular subtype of prostate cancer;
b) a genomic classifier score of greater than about 0.6;
c) an increased expression of at least one signature of Class One co-regulated signatures;
d) an increased expression of at least one signature of Class Two co-regulated signatures;
e) a decreased expression of at least one signature of Class Three co-regulated signatures;
f) an increased expression of at least one signature of Class Four co-regulated signatures; or a combination thereof.

Another aspect of the invention relates to methods of treating prostate cancer (e.g., nmCRPC) in a human male, comprising, consisting of and/or consisting essentially of:

administering a therapeutically effective amount of an androgen-receptor inhibitor (e.g., APA) and a therapeutically effective amount of an approved drug product that contains an androgen deprivation therapy (ADT) (e.g., APA+ADT) to the human male if a biological sample obtained from the human male is determined to have:
a) a luminal-like or a basal-like molecular subtype of prostate cancer;
b) a genomic classifier score of greater than about 0.6;
c) an increased expression of at least one signature of Class One co-regulated signatures;
d) an increased expression of at least one signature of Class Two co-regulated signatures;
e) a decreased expression of at least one signature of Class Three co-regulated signatures;
f) an increased expression of at least one signature of Class Four co-regulated signatures; or a combination thereof.

Another aspect of the invention relates to methods of predicting a human male having a non-metastatic castration resistant prostate cancer (nmCRPC) to have an improved benefit from administration of a therapeutically effective amount of an androgen-receptor inhibitor (e.g., APA) and a therapeutically effective amount of an approved drug product that contains an androgen deprivation therapy (ADT) (e.g., APA+ADT) relative to sole administration of a therapeutically effective amount of the ADT, said method comprising, consisting of and/or consisting essentially of:
a) determining if a biological sample obtained from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof, and
b) predicting that the human male to have an improved benefit from administration of the therapeutically effective amount of the androgen-receptor inhibitor (e.g., APA) and the therapeutically effective amount of the ADT relative to sole administration of the therapeutically effective amount of the ADT based on:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof.

Another aspect of the invention relates to methods of improving response to treating non-metastatic castration resistant prostate cancer (nmCRPC) in a human male using a combined administration of a therapeutically effective amount of an androgen-receptor inhibitor (e.g., APA) and a therapeutically effective amount of an approved drug product that contains an androgen deprivation therapy (ADT) (e.g., APA+ADT) relative to sole administration of a therapeutically effective amount of the ADT, the method comprising, consisting of and/or consisting essentially of:
a) determining if a biological sample obtained from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof, and
b) improving response to combined administration of the therapeutically effective amount of the androgen-receptor inhibitor (e.g., APA) and the therapeutically effective amount of the ADT relative to sole administration of the therapeutically effective amount of the ADT, based on:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof.

Another aspect of the invention relates to methods of identifying a human male (or a subset of human males) diagnosed with nmCRPC, wherein the nmCRPC is predicted to have an improved treatment benefit from a therapeutically effective amount of an androgen-receptor inhibitor (e.g., APA) and a therapeutically effective amount of an androgen deprivation therapy (ADT) (e.g., APA+ADT) relative to sole administration of a therapeutically effective amount of the ADT, comprising, consisting of and/or consisting essentially of:
a) determining if a biological sample obtained from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof, and
b) predicting that the human male to have an improved benefit from administration of the therapeutically effective amount of the androgen-receptor inhibitor (e.g., APA) and the therapeutically effective amount of the ADT relative to sole administration of the therapeutically effective amount of the ADT based on:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures,
   or a combination thereof.

Another aspect of the invention relates to methods of predicting an improvement of treatment response of nmCRPC to combined administration of a therapeutically effective amount of an androgen-receptor inhibitor (e.g., APA) and a therapeutically effective amount of an androgen deprivation therapy (ADT) (e.g., APA+ADT) relative to sole administration of a therapeutically effective amount of the ADT in a human male, comprising, consisting of and/or consisting essentially of:
a) determining if a biological sample obtained from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof, and
b) predicting an improvement of response to combined administration of the therapeutically effective amount of the androgen-receptor inhibitor (e.g., APA) and the therapeutically effective amount of the ADT relative to sole administration of the therapeutically effective amount of the ADT, based on:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof.

Another aspect of the invention relates to methods of estimating clinical outcome in a human male having cancer (e.g., nmCRPC) and receiving APA+ADT, comprising, consisting of and/or consisting essentially of:
a) obtaining gene expression data of a biological sample obtained from the human male;
b) estimating that the human male to receive improved benefit from APA+ADT compared to ADT alone if the biological sample has:
   i) a basal-like or luminal-like molecular subtype of prostate cancer;
   ii) a genomic classifier score of greater than about 0.6;
   iii) an increased expression of at least one signature of Class One, Class Two, and/or Class Four co-regulated signatures;
   iv) a decreased expression of at least one signature of Class Three co-regulated signatures;
   or any combination thereof.

Another aspect of the invention relates to methods of predicting a clinical outcome of treatment of cancer (e.g., nmCRPC) in a human male with ADT+APA, comprising, consisting of and/or consisting essentially of:
a) obtaining expression data in a biological sample obtained from the human male;
b) assigning the expression data to co-regulated signatures selected from the group consisting of: Prognosis Related Signatures, Steroid Homeostasis Related Signatures, Hormonal Therapy Non-Responsive Basal and Neuroendocrine Like Signatures, and Immune and Stromal IL2/IL-6-JAK-STAT5 Signatures, and combinations thereof;
c) determining an ADT+APA score for the biological sample; and
d) predicting the clinical outcome of the treatment based on the expression level of at least one class.

In some embodiments, the prostate cancer is non-metastatic castration resistant prostate cancer (nmCRPC). In some embodiments, the human male has chemotherapy-naive metastatic castration-resistant prostate cancer.

In some embodiments, the nmCRPC is a high risk nmCRPC. In some embodiments, the high risk nmCRPC has a prostate specific antigen doubling time (PSADT) of less than about 20 months, e.g., less than about 19 months, less than about 18 months, less than about 17 months, less than about 16 months, less than about 15 months, less than about 14 months, less than about 13 months, less than about 12 months, less than about 11 months, less than about 9 months, less than about 8 months, less than about 7 months, less than about 6 months, less than about 5 months, less than about 4 months, less than about 3 months, less than about 2 months, or less than about 1 month. In some embodiments, the high risk nmCRPC has a PSADT of less than about 10 months.

In some embodiments, the high risk nmCRPC has a PSADT of between about 1 and about 20 months, for example, about 1-19 months, about 2-19 months, about 2-18 months, about 3-18 months, about 3-17 months, about 4-17 months, about 4-16 months, about 5-16 months, about 5-15 months, about 6-15 months, about 6-14 months, about 7-14 months, about 7-13 months, about 8-13 months, about 8-12 months, about 9-12 months, or about 9-11 months.

In some embodiments, the high risk nmCRPC has local-regional recurrence (e.g., primary tumor bed, bladder neck, anastomotic area, pelvic lymph nodes). In some embodiments, the high risk nmCRPC has a high Gleason score. In some embodiments, the high risk nmCRPC has bulky tumor.

In some embodiments, the method further comprises obtaining the biological sample from the human male.

In some embodiments, the human male has undergone a prostatectomy.

In some embodiments, the biological sample is a primary prostate tumor sample.

In some embodiments, the biological sample is a prostate biopsy sample.

A biopsy is a procedure to remove tissue (e.g., suspicious tissue) or a sample of cells from a living body of a human male, e.g., from a human male's prostate. Prostate biopsy samples can be collected in different ways. The prostate biopsy may involve passing a needle through the wall of the rectum (transrectal biopsy). This is the most common way of performing a prostate biopsy. Another method of collecting the prostate biopsy sample can include inserting a needle through the area of skin between the anus and scrotum (transperineal biopsy). A small cut is made in the area of skin (perineum) between the anus and the scrotum. The biopsy needle is inserted through the cut and into the prostate to draw out a sample of tissue. An MRI or CT scan is generally used to guide this procedure. A physician may target a suspicious area to biopsy or may take samples from several places in the prostate. Generally, 10 to 12 tissue samples are taken. As such, in embodiments of the invention, the prostate biopsy sample may include normal prostate tissue, normal prostate tissue and cancerous tissue, or only cancerous tissue.

In some embodiments, the biological sample is a surgical tumor sample. A surgical tumor sample can include a prostate sample that is collected during a prostatectomy. A surgical tumor sample can include a tumor or metastatic lesions that are remote to the prostate. A surgical tumor sample can include the whole prostate or a portion of the prostate. In some embodiments, the surgical tumor sample comprises a tumor.

In some embodiments, the biological sample obtained from the human male is determined to have a molecular subtype of prostate cancer selected from a luminal-like molecular subtype or a basal-like molecular subtype. In some embodiments, the biological sample has a luminal-like molecular subtype of prostate cancer. In some embodiments, the biological sample has a basal-like molecular subtype of prostate cancer.

In some embodiments, whether the biological sample comprises cells of a basal-like or luminal-like subtype is determined based on mRNA expression, one or more genetic markers associated with each subtype, or a combination thereof using techniques such as Northern blot analysis, Southern blot analysis, Western blot analysis, microarray, etc.

In some embodiments, whether the biological sample comprises cells of a basal-like or luminal-like subtype is determined based on the histological features of the cells, e.g., microscopic analysis using Hematoxylin and eosin staining (H&E), immunohistochemistry, or a combination thereof. Standard light microscopy, and/or software analysis can be used. In some embodiments, a gross analysis of the surgical tumor sample or prostate biopsy sample is used.

In some embodiments, the genomic classifier (GC) score is determined. A GC score represents a continuous score of 0-1. Patients with score >0.6 appear to have a higher risk for progression to metastasis (Klein EA et al., European Urology 67(4):778-86 (2015)).

In some embodiments, the human male (having nmCRPC) is determined to have a high risk of metastasis based on the GC score of greater than about 0.6. In some embodiments, the human male (having nmCRPC) is determined to have a high risk of metastasis based on the GC score of greater than 0.6. In some embodiments, a biological sample having a GC score of above about 0.6 and a poor prognosis with ADT alone predicts that the human male benefits from ADT+APA. In some embodiments, a biological sample having a GC score of less than about 0.6 predicts that the human male benefits from ADT and ADT+APA.

In one embodiment, the genomic classifier is a 22-marker genomic classifier (e.g., DECIPHER®) comprising markers corresponding to RNA associated with the following genes/loci (nearest gene/locus (type of marker; cytoband)): LASP1 (coding, 17q12), IQGAP3 (3′ UTR, 1q23.1), NFIB (intronic, 9p23), S1PR4 (3′ UTR, 19p13.3), THBS2 (3′ UTR, 6q27), ANO7 (3′ UTR, 2q37.3), PCDH7 (intronic, 4p15.1), MYBPC1 (coding, 12q23.2), EPPK1 (3′ UTR, 8q24.3), TSBP (intronic, 6p21.32), PBX1 (coding, 1q23.3), NUSAP1 (3′ UTR, 15q15.1), ZWILCH (3′ UTR, 15q22.31), UBE2C (3′ UTR, 20q13.12), CAMKC2N1 (coding antisense, 1p36.12), RABGAP1 (exon/intron junction antisense, 9q33.2), PCAT-32 (non-coding transcript, 5p15.2), GYATL1P4/PCAT-80 (non-coding transcript, 11q12.1) and TNFRSF19 (intronic, 13q12.12) (Erho N et al., PLoS ONE 8(6): e66855 (2013), incorporated herein by reference in its entirety).

In some embodiments, the genomic classifier comprises at least one marker selected from the group consisting of: LASP1, IQGAP3, NFIB, S1PR4, THBS2, ANO7, PCDH7, MYBPC1, EPPK1, TSBP, PBX1, NUSAP1, ZWILCH, UBE2C, CAMKC2N1, RABGAP1, PCAT-32, GYATL1P4/PCAT-80, TNFRSF19, and combinations thereof

In some embodiments, one marker is used to determine the GC score. In other embodiments, 2-22 markers are used to determine the GC score, e.g., 3-22, 3-20, 4-20, 4-18, 5-18, 5-16, 6-16, 6-14, 7-14, 7-12, 8-12, or 8-10 markers are used to determine the GC score. In some embodiments, 22 markers are used to determine the GC score.

In some embodiments, the expression level of at least one signature of Class One, Class Two, Class Three, and/or Class Four co-regulated signatures of the biological sample is determined. In some embodiments, the biological sample is determined to have:
a) an increased expression of at least one signature of Class One co-regulated signatures;
b) an increased expression of at least one signature of Class Two co-regulated signatures;
c) a decreased expression of at least one signature of Class Three co-regulated signatures;
d) an increased expression of at least one signature of Class Four co-regulated signatures; or any combination thereof.

In some embodiments, the gene signature is a Decipher gene signature. In some embodiments, the at least one signature of the Class One co-regulated signatures is a signature in Table 4. In some embodiments, the at least one signature of the Class Two co-regulated signatures is a signature in Table 5. In some embodiments, the at least one signature of the Class Three co-regulated signatures is a signature in Table 6. In some embodiments, the at least one signature of the Class Four co-regulated signatures is a signature in Table 7.

In some embodiments, discriminant analysis (DA) and logistic regression are used to score the expression profile of a biological sample and determine the human male's (patient's) clinical outcome based on the score. DA is statistical tool for classifying cases into the values of a categorical dependent variable, usually dichotomized.

In some embodiments, the function is generated using the censoring information on a patient positive or negative for metastasis, which is equivalent to higher or less risk. In some embodiments, the discriminant scores with respect to observed signature scores for each human male is recorded to classify them as positive or negative.

In some embodiments, the computed discriminant score is used to establish a cutoff score for assigning a human male to a group. For example, if a human male's discriminant score is higher than or equal to the cutoff score, the human male is assigned to group 1 (positive), otherwise the human male is assigned to group 2 (negative).

DA is an earlier alternative to logistic regression, which is now frequently used in place of DA as it usually involves fewer violations of assumptions (independent variables needn't be normally distributed, linearly related, or have equal within-group variances), is robust, handles categorical as well as continuous variables, and has coefficients which many find easier to interpret (McLachlan and Geoffrey J., Discriminant analysis and statistical pattern recognition. NY: Wiley-Interscience. 2004 (Wiley Series in Probability and Statistics)).

With logistic regression a signature score can determine a patient's outcome. Like DA, in logistic regression the outcome is measured with a dichotomous variable (positive or negative for metastasis), and it can also be used as classifier since the cutoff value can be adjusted given the predicted probability to be used in classification.

In some embodiments, the biological sample is assigned to the high expression group (e.g., of Class One, Two, Three, or Four signatures) if the expression level is above or equal to median. In some embodiments, the biological sample is assigned to the low expression group (e.g., of Class One, Two, Three, or Four signatures) if the expression level is below median.

In some embodiments, the biological sample is determined to have an increased expression of at least one signature of the Class One co-regulated signatures.

In some embodiments, the at least one signature of the Class One co-regulated signatures is selected from the group consisting of: age112012_1, bibikova2007_1, bismar2006_1, bismar2017_1, cheville2008_1, cuzick2011_1, cuzick2011_lm_1, decipher_1, decipherv2_2, genomic_capras_1, genomic_gleason_grade_1, genomic_gleason_grade_2, glinsky2005_1, hallmark_mtorc1_signaling, hallmark_myc_targets_v1, hallmark_myc_targets_v2, klein2014_1, lapointe2004_1, larkin2012_1, long2014_1, nakagawa2008_1, non_organ_confined_1, normaltumor_1, pam50_luminalB, penney2011_1, penney2011_lm_1, ramaswamy2003_1, saa12007_1, saa12007_pten, sdms_1, singh2002_1, staging_epe_1, staging_1ni_1, staging_svi_1, stephenson2005_1, talantov2010_1, varambally2005_1, wu2013_1, yu2007_1, and combinations thereof.

In some embodiments, a patient has an increased expression of at least one signature of the Class One co-regulated signatures if the patient's expression score on the at least one signature of the Class One co-regulated signatures is higher than or equal to the median expression score on said signature in a population of nmCRPC patients.

In some embodiments, the at least one signature of the Class One co-regulated signatures comprises genomic_gleason_grade_2. In some embodiments, the at least one signature of the Class One co-regulated signatures has an increased expression if the expression score (normalized signature score) is higher than or equal to 0.49.

In some embodiments, at least 2, 3, 4, 5, 6, 7, 8, 9, 10 or more signatures of the Class One co-regulated signatures are used to determine if the biological sample has an increased expression of the Class One co-regulated signatures.

In some embodiments, the biological sample is determined to have an increased expression of at least one signature of the Class Two co-regulated signatures.

In some embodiments, the at least one signature of the Class Two co-regulated signatures is selected from the group consisting of: ar_related_pathway_Ar7, ar_related_pathway_glucocorticoid_receptor, aros_1, docetaxel_sens_1, ergmodel_1, glinsky2004_1, hallmark_adipogenesis, hallmark_androgen_response, hallmark_angiogenesis_Brauer2013, hallmark_angiogenesis_KeggVEGF, hallmark_angiogenesis_Liberzon2015, hallmark_angiogenesis_Masiero2013, hallmark_angiogenesis_Nolan2013, hallmark_angiogenesis_Uhlik2016, hallmark_apical_surface, hallmark_bile_acid_metabolism, hallmark_cholesterol_homeostasis, hallmark_dna_repair, hallmark_e2f_targets, hallmark_fatty_acid_metabolism, hallmark_g2m_checkpoint, hallmark_glycolysis, hallmark_hedgehog_signaling, hallmark_heme_metabolism, hallmark_mitotic_spindle, hallmark_notch_signaling, hallmark_oxidative_phosphorylation, hallmark_peroxisome, hallmark_pi3k_akt_mtor_signaling, hallmark_protein_secretion, hallmark_spermatogenesis, hallmark_unfolded_protein_response, hallmark_uv_response_dn, hallmark_xenobiotic_metabolism, immunophenoscore_1_CP, immunophenoscore_1_CTLA.4, immunophenoscore_1_IDO1, immunophenoscore_1_LAG3, immunophenoscore_1_PD.1, immunophenoscore_1_PD.L2, immunophenoscore_1_Tem.CD4, immunophenoscore_1_TIGIT, kegg_mismatch_repair, kegg_non_homologous_end_joining, kegg_nucleotide_excision_repair, long2011_1, nelson_2016_AR_1, pam50_luminalA, pea_vs_mibc_1, race_1, ragnum2015_1, and combinations thereof.

In some embodiments, a patient has an increased expression of at least one signature of the Class Two co-regulated signatures if the patient's expression score on the at least one signature of the Class Two co-regulated signatures is higher than or equal to the median expression score on said signature in a population of nmCRPC patients.

In some embodiments, the at least one signature of the Class Two co-regulated signatures comprises hallmark_cholesterol_homeostasis. In some embodiments, the at least one signature of the Class Two co-regulated signatures has an increased expression if the expression score (normalized signature score) is higher than or equal to 0.25.

Hallmark_cholestrol_homeostasis includes: ABCA2, ACAT2, ACSS2, ACTG1, ADH4, ALCAM, ALDOC, ANTXR2, ANXA13, ANXA5, ATF3, ATF5, ATXN2, AVPR1A, CBS, CD9, CHKA, CLU, CPEB2, CTNNB1, CXCL16, CYP51A1, DHCR7, EBP, ECH1, ERRFI1, ETHE1, FABP5, FADS2, FAM129A, FASN, FBXO6, FDFT1, FDPS, GLDC, GNAI1, GPX8, GSTM2, GUSB, HMGCR, HMGCS1, HSD17B7, IDI1, JAG1, LDLR, LGALS3, LGMN, LPL, LSS, MAL2, MVD, MVK, NFIL3, NSDHL, PCYT2, PDK3, PLAUR, PLSCR1, PMVK, PNRC1, PPARG, S100A11, SC5DL, SCD, SEMA3B, SQLE, SREBF2, STARD4, STX5, TM7SF2, TMEM97, TNFRSF12, TP53INP1 and TRIB3.

In some embodiments, at least 2, 3, 4, 5, 6, 7, 8, 9, 10 or more signatures of the Class Two co-regulated signatures are used to determine if the biological sample has an increased expression of the Class Two co-regulated signatures.

In some embodiments, the biological sample is determined to have a decreased expression of at least one signature of the Class Three co-regulated signatures.

In some embodiments, the at least one signature of the Class Three co-regulated signatures is selected from the group consisting of: ars_1, beltran2016_1, dasatinib_sens_1, estimate2013_2_purity, hallmark_apical_junction, hallmark_apoptosis, hallmark_coagulation, hallmark_epithelial_mesenchymal_transition, hallmark_estrogen_response_early, hallmark_estrogen_response_late, hallmark_hypoxia, hallmark_kras_signaling_dn, hallmark_myogenesis, hallmark_p53_pathway, hallmark_pancreas_beta_cells, hallmark_reactive_oxigen_species_pathway, hallmark_tgf_beta_signaling, hallmark_tnfa_signaling_via_nfkb, hallmark_uv_response_up, hallmark_wnt_beta_catenin_signaling, immunophenoscore_1_ICOS, immunophenoscore_1_MDSC, immunophenoscore_1_PD.L1, immunophenoscore_1_SC, immunophenoscore_1_TIM3, immunophenoscore_1_Treg, kegg_base_excision_repair, kegg_homologous_recombination, lotan2016_1, neg_ctrl_qc, nelson2016_1, pam50_basal, portos_1, portos_2, rbloss_1, smallcell_1, smallcell_2, smallcell_3, torresroca2009_1, zhang2016_basal_1, and combinations thereof.

In some embodiments, a patient has a decreased expression of at least one signature of the Class Three co-regulated signatures if the patient's expression score on the at least one signature of the Class Three co-regulated signatures is lower than the median expression score on said signature in a population of nmCRPC patients.

In some embodiments, the at least one signature of the Class Three co-regulated signatures comprises beltran2016_1. In some embodiments, the at least one signature of the Class Three co-regulated signatures has a decreased expression if the expression score (normalized signature score) is lower than −0.44.

Beltran2016_1 includes: MPHOSPH9, ADAM7, FOH1, CD200, FKBP5, GLRA2, NDRG1, CAMKK2, MAN1A1, MED28, ELL2, ACSL3, PMEPA1, GNMT, ABCC4, HERC3, PIP4K2B, KLK3, EAF2, CENPN, MAPRE2, NKX3-1, KLK2, AR, TNK1, MAF, C1ORF116, TMPRSS2, TBC1D9B and ZBTB10.

In some embodiments, at least 2, 3, 4, 5, 6, 7, 8, 9, 10 or more signatures of the Class Three co-regulated signatures are used to determine if the biological sample has a decreased expression of the Class Three co-regulated signatures.

In some embodiments, the biological sample is determined to have increased expression of at least one signature of the Class Four co-regulated signatures.

In some embodiments, the at least one signature of the Class Four co-regulated signatures is selected from the group consisting of: estimate2013_2_estimate, estimate2013_2_immune, estimate2013_2_stromal, hallmark_allograft_rejection, hallmark_angiogenesis, hallmark_complement, hallmark_IL2_JAK_STAT5_signaling, hallmark_IL6_JAK_STAT3_signaling, hallmark_inflammatory_response, hallmark_interferon_alpha_response, hallmark_interferon_gamma_response, hallmark_kras_signaling_up, immunophenoscore_1_Act.CD4, immunophenoscore_1_Act.CD8, immunophenoscore_1_B2M, immunophenoscore_1_CD27, immunophenoscore_1_EC, immunophenoscore_1_HLA.A, immunophenoscore_1_HLA.B, immunophenoscore_1_HLA.C, immunophenoscore_1_HLA.DPA1, immunophenoscore_1_HLA.DPB1, immunophenoscore_1_HLA.E, immunophenoscore_1_HLA.F, immunophenoscore_1_IPS, immunophenoscore_1_IPS.raw, immunophenoscore_1_MHC, immunophenoscore_1_TAP1, immunophenoscore_1_TAP2, immunophenoscore_1_Tem.CD8, and combinations thereof.

In some embodiments, a patient has an increased expression of at least one signature of the Class Four co-regulated signatures if the patient's expression score on the at least one signature of the Class Four co-regulated signatures is higher than or equal to the median expression score on said signature in a population of nmCRPC patients.

In some embodiments, the at least one signature of the Class Four co-regulated signatures comprises hallmark_IL2_JAK_STAT5_signaling. In some embodiments, the at least one signature of the Class Four co-regulated signatures has an increased expression if the expression score (normalized signature score) is higher than or equal to −0.42.

Hallmark_IL2_JAK_STAT5_signaling includes: ABCB1, ADAM19, AGER, AHCY, AHNAK, AHR, AKAP2, ALCAM, AMACR, ANXA4, APLP1, ARL4A, BATF, BATF3, BCL2, BCL2L1, BHLHE40, BMP2, BMPR2, CA2, CAPG, CAPN3, CASP3, CCND2, CCND3, CCNE1, CCR4, CD44, CD48, CD79B, CD81, CD83, CD86, CDC42SE2, CDC6, CDCP1, CDKN1C, CISH, CKAP4, COCH, COL6A1, CSF1, CSF2, CST7, CTLA4, CTSZ, CXCL10, CYFIP1, DCPS, DENND5A, DHRS3, DRC1, ECM1, EEF1AKMT1, EMP1, ENO3, ENPP1, EOMES, ETFBKMT, ETV4, F2RL2, FAH, FAM126B, FGL2, FLT3LG, FURIN, GABARAPL1, GADD45B, GALM, GATA1, GBP4, GLIPR2, GPR65, GPR83, GPX4, GSTO1, GUCY1B1, HIPK2, HK2, HOPX, HUWE1, ICOS, IFITM3, IFNGR1, IGF1R, IGF2R, IKZF2, IKZF4, IL10, ILlORA, IL13, IL18R1, IL1R2, IL1RL1, IL2RA, IL2RB, IL3RA, IL4R, IRF4, IRF6, IRF8, ITGA6, ITGAE, ITGAV, ITIH5, KLF6, LCLAT1, LIF, LRIG1, LRRC8C, LTB, MAFF, MAP3K8, MAP6, MAPKAPK2, MUC1, MXD1, MYC, MYO1C, MYO1E, NCOA3, NCS1, NDRG1, NFIL3, NFKBIZ, NOP2, NRP1, NT5E, ODC1, P2RX4, P4HA1, PDCD2L, PENK, PHLDA1, PHTF2, PIM1, PLAGL1, PLEC, PLIN2, PLPP1, PLSCR1, PNP, POU2F1, PRAF2, PRKCH, PRNP, PTCH1, PTGER2, PTH1R, PTRH2, PUS1, RABGAP1L, RGS16, RHOB, RHOH, RNH1, RORA, RRAGD, S100A1, SCN9A, SELL, SELP, SERPINB6, SERPINC1, SH3BGRL2, SHE, SLC1A5, SLC29A2, SLC2A3, SLC39A8, SMPDL3A, SNX14, SNX9, SOCS1, SOCS2, SPP1, SPRED2, SPRY4, ST3GAL4, SWAP70, SYNGR2, SYT11, TGM2, TIAM1, TLR7, TNFRSF18, TNFRSF1B, TNFRSF21, TNFRSF4, TNFRSF8, TNFRSF9, TNFSF10, TNFSF11, TRAF1, TTC39B, TWSG1, UCK2, UMPS, WLS and XBP1.

In some embodiments, at least 2, 3, 4, 5, 6, 7, 8, 9, 10 or more signatures of the Class Four co-regulated signatures are used to determine if the biological sample has a decreased expression of the Class Four co-regulated signatures.

In some embodiments, identifying the co-regulated expression signatures comprises applying consensus clustering and determining the co-regulated expression signatures based in part on a relevant consensus cluster.

In some embodiments, identifying the co-regulated expression signatures comprises scoring the signatures to create signature scores, ranking the signatures by size of signature score to create ranked signatures, transposing the ranked signatures, and performing quantile normalization over the samples.

In some embodiments, evaluating the expression signatures comprises using Kaplan-Meier analysis, cox proportional modelling or both Kaplan-Meier analysis and cox proportional modelling.

In some embodiments, the methods further comprise stratifying the patients into high and low expression groups based for each class of co-regulated expression signatures, and evaluating the expression signatures for association between levels of expression and interaction of administration and outcome for the high expression groups and for the low expression groups.

In some embodiments, the human male receives a combined administration of APA+ADT. The SPARTAN trial demonstrated that the addition of APA to androgen deprivation therapy (ADT) improved metastasis-free survival (MFS) and second progression-free survival (PFS2) in nmCRPC patients.

In some embodiments, the improved benefit comprises an increase in metastasis-free survival (MFS), an increase in time to metastasis (TTM), an increase in second progression-free survival (PFS2), an increase in time to symptomatic progression, an increase in time to initiation of cytotoxic chemotherapy, a delay in symptoms related to disease progression, an improvement in overall survival, survival benefit, or a combination thereof.

In some embodiments, the improved benefit comprises an increase in MFS. In some embodiments, MFS of combined administration of APA+ADT is improved relative to sole administration of ADT alone.

In some embodiments, the increase in the MFS is about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months.

In some embodiments, the increase in the MFS is at least about 1 month, e.g., at least about 2 months, at least about 3 months, at least about 4 months, at least about 5 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, or at least about 24 months. In some embodiments, the increase in the MFS is at least about 6 months.

In some embodiments, the increase in the MFS is between about 1 month and about 48 months, e.g., about 1-45 months, about 2-45 months, about 2-42 months, about 3-42 months, about 3-39 months, about 4-39 months, about 4-36 months, about 5-36 months, about 5-33 months, about 6-33 months, about 6-30 months, about 7-30 months, about 7-27 months, about 8-27 months, about 8-24 months, about 9-24 months, about 9-21 months, about 10-21 months, about 10-18 months, about 11-18 months, about 11-15 months, or about 12-15 months.

In some embodiments, the increase in the MFS is relative to the mean survival rate of a population of male humans having nmCRPC and having been treated with a placebo.

In some embodiments, the MFS refers to the time from randomization to the time of first evidence of BICR-confirmed bone or soft tissue distant metastasis or death due to any cause, whichever occurs first.

In some embodiments, the improved benefit comprises an increase in PFS2. In some embodiments, PFS2 of combined administration of APA+ADT is improved relative to sole administration of ADT alone.

In some embodiments, the increase in the PFS2 is about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months.

In some embodiments, the increase in the PFS2 is at least about 1 month , e.g., at least about 2 months, at least about 3 months, at least about 4 months, at least about 5 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, or at least about 24 months. In some embodiments, the increase in the PFS2 is at least about 6 months.

In some embodiments, the increase in the PFS2 is between about 1 month and about 48 months, e.g., about 1-45 months, about 2-45 months, about 2-42 months, about 3-42 months, about 3-39 months, about 4-39 months, about 4-36 months, about 5-36 months, about 5-33 months, about 6-33 months, about 6-30 months, about 7-30 months, about 7-27 months, about 8-27 months, about 8-24 months, about 9-24 months, about 9-21 months, about 10-21 months, about 10-18 months, about 11-18 months, about 11-15 months, or about 12-15 months.

In some embodiments, the improved benefit comprises an increase in time to metastasis (TTM).

In some embodiments, the increase in the TTM is about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months.

In some embodiments, the increase in the TTM is at least about 1 month , e.g., at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, or at least about 24 months.

In some embodiments, the increase in the TTM is between about 1 month and about 48 months, e.g., about 1-45 months, about 2-45 months, about 2-42 months, about 3-42 months, about 3-39 months, about 4-39 months, about 4-36 months, about 5-36 months, about 5-33 months, about 6-33 months, about 6-30 months, about 7-30 months, about 7-27 months, about 8-27 months, about 8-24 months, about 9-24 months, about 9-21 months, about 10-21 months, about 10-18 months, about 11-18 months, about 11-15 months, or about 12-15 months.

In some embodiments, the improved benefit comprises a delay in symptoms related to disease progression.

In some embodiments, the androgen-receptor inhibitor (i.e., antiandrogen) is a small molecule. In some embodiments, the androgen-receptor inhibitor is an androgen receptor (AR) antagonist. In some embodiments, the androgen-receptor inhibitor is an AR full antagonist. In some embodiments, the androgen-receptor inhibitor is APA+ADT. In some embodiments, the administering of the androgen-receptor inhibitor (e.g., APA+ADT) is by oral administration.

Androgen-deprivation therapy, or ADT, refers to the reduction of androgen levels in a prostate cancer patient to castrated levels of testosterone (about <50 ng/dL). In some embodiments, such treatments can include orchiectomy or the use of gonadotropin-releasing hormone agonists or antagonists. In some embodiments, ADT includes surgical castration (orchiectomy) and/or the administration of luteinizing hormone-releasing hormone (“LHRH”) agonists to a human. Examples of LHRH agonists include goserelin acetate, histrelin acetate, leuprolide acetate, and triptorelin palmoate.

Physicians can prescribe LHRH agonists in accordance with instructions, recommendations and practices. In some embodiments, this includes about 0.01 mg to about 20 mg of goserelin acetate over a period of about 28 days to about 3 months, about 3.6 mg to about 10.8 mg of goserelin acetate over a period of about 28 days to about 3 months; about 0.01 mg to about 200 mg of leuprolide acetate over a period of about 3 days to about 12 months, preferably about 3.6 mg of leuprolide acetate over a period of about 3 days to about 12 months; or about 0.01 mg to about 20 mg of triptorelin palmoate over a period of about 1 month, preferably about 3.75 mg of triptorelin palmoate over a period of 1 month. In some embodiments, this includes about 50 mg of histrelin acetate over a period of 12 months of histrelin acetate or about 50 μg per day of histrelin acetate.

Androgen depletion is the standard treatment with a generally predictable outcome: decline in PSA, a period of stability in which the tumor does not proliferate, followed by rising PSA and regrowth as castration-resistant disease. Historically, ADT has been the standard of care for patients with metastatic prostate cancer.

The administration of the therapeutics described herein may be carried out in any manner, e.g., by parenteral or nonparenteral administration, including by aerosol inhalation, injection, infusions, ingestion, implantation or transplantation. For example, the compositions described herein may be administered to a patient trans-arterially, intradermally, subcutaneously, intratumorally, intramedullary, intranodally, intramuscularly, by intravenous (i.v.) injection, or intraperitoneally. In one aspect, the compositions of the present disclosure are administered by i.v. injection. In one aspect, the compositions of the present disclosure are administered to a human male by intradermal or subcutaneous injection. The compositions may be injected, for instance, directly into a tumor, lymph node, tissue, or organ.

In some embodiments of the invention, the administering is by oral administration. In one embodiment, the compositions (e.g., APA and/or androgen deprivation therapy components) are present in a solid oral dosage form. In some embodiments, the composition is formulated as a tablet. In some embodiments, the androgen deprivation therapy is enzalutamide. Solid oral dosage forms containing either apalutamide or enzalutamide may be provided as soft gel capsules, as disclosed in WO2014113260 and CN104857157, each of which is incorporated herein by reference, or as tablets as disclosed in WO2016090098, WO2016090101, WO2016090105, and WO2014043208, each of which is incorporated herein by reference. Techniques suitable for preparing solid oral dosage forms of the present invention are described in Remington's Pharmaceutical Sciences, 18th edition, edited by AR. Gennaro, 1990, Chapter 89, and in Remington—The Science, and Practice of Pharmacy, 21st edition, 2005, Chapter 45.

To prepare pharmaceutical compositions, the active pharmaceutical ingredient can be admixed with a pharmaceutical carrier according to conventional pharmaceutical compounding techniques, which carrier may take a wide variety of forms depending of the form of preparation desired for administration (e.g., oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

In solid oral preparations such as, for example, dry powders for reconstitution or inhalation, granules, capsules, caplets, gelcaps, pills and tablets (each including immediate release, timed release and sustained release formulations), suitable carriers and additives include but are not limited to diluents, granulating agents, lubricants, binders, glidants, disintegrating agents and the like. Because of their ease of administration, tablets and capsules represent an advantageous oral dosage unit form, in which case solid pharmaceutical carriers are obviously employed. If desired, tablets may be sugar coated, gelatin coated, film coated or enteric coated by standard techniques.

In some embodiments, the compositions utilized by the methods described are in unit dosage forms from such as tablets, pills, capsules, dry powders for reconstitution or inhalation, granules, lozenges, sterile solutions or suspensions, metered aerosol or liquid sprays, drops, or suppositories for administration by oral, intranasal, sublingual, intraocular, transdermal, rectal, dry powder inhaler or other inhalation or insufflation means. These formulations are manufactured by conventional formulation techniques. For preparing solid pharmaceutical compositions such as tablets, the principal active ingredient is mixed with a pharmaceutical carrier, e.g., conventional tableting ingredients such as diluents, binders, adhesives, disintegrants, lubricants, antiadherents, and glidants. Suitable diluents include, but are not limited to, starch (i.e. corn, wheat, or potato starch, which may be hydrolyzed), lactose (granulated, spray dried or anhydrous), sucrose, sucrose-based diluents (confectioner's sugar; sucrose plus about 7 to 10 weight percent invert sugar; sucrose plus about 3 weight percent modified dextrins; sucrose plus invert sugar, about 4 weight percent invert sugar, about 0.1 to 0.2 weight percent cornstarch and magnesium stearate), dextrose, inositol, mannitol, sorbitol, microcrystalline cellulose (i.e., AVICEL microcrystalline cellulose available from FMC Corp.), dicalcium phosphate, calcium sulfate dihydrate, calcium lactate trihydrate and the like. Suitable binders and adhesives include, but are not limited to acacia gum, guar gum, tragacanth gum, sucrose, gelatin, glucose, starch, and cellulosics (i.e. methylcellulose, sodium carboxymethylcellulose, ethylcellulose, hydroxypropylmethylcellulose, hydroxypropylcellulose, and the like), water soluble or dispersible binders (i.e., alginic acid and salts thereof, magnesium aluminum silicate, hydroxyethylcellulose [i.e., TYLOSE available from Hoechst Celanese], polyethylene glycol, polysaccharide acids, bentonites, polyvinylpyrrolidone, polymethacrylates and pregelatinized starch) and the like. Suitable disintegrants include, but are not limited to, starches (corn, potato, etc.), sodium starch glycolates, pregelatinized starches, clays (magnesium aluminum silicate), celluloses (such as crosslinked sodium carboxymethylcellulose and microcrystalline cellulose), alginates, pregelatinized starches (i.e. corn starch, etc.), gums (i.e. agar, guar, locust bean, karaya, pectin, and tragacanth gum), cross-linked polyvinylpyrrolidone and the like. Suitable lubricants and anti-adherents include, but are not limited to, stearates (magnesium, calcium and sodium), stearic acid, talc waxes, stearowet, boric acid, sodium chloride, DL-leucine, carbowax 4000, carbowax 6000, sodium oleate, sodium benzoate, sodium acetate, sodium lauryl sulfate, magnesium lauryl sulfate and the like. Suitable gildants include, but are not limited to, talc, cornstarch, silica (i.e. CAB-O-SIL silica available from Cabot, SYLOID silica available from W. R. Grace/Davison, and AEROSIL silica available from Degussa) and the like. Sweeteners and flavorants may be added to chewable solid dosage forms to improve the palatability of the oral dosage form. Additionally, colorants and coatings may be added or applied to the solid dosage form for ease of identification of the drug or for aesthetic purposes. These carriers are formulated with the pharmaceutical active to provide an accurate, appropriate dose of the pharmaceutical active with a therapeutic release profile.

Binders suitable for use in the pharmaceutical compositions utilized herein include, but are not limited to, starches, cellulose, and its derivatives (e.g., ethylcellulose, cellulose acetate, carboxymethyl cellulose calcium, sodium carboxymethyl cellulose, methylcellulose, hydroxypropyl methylcellulose), polyviny pyrrolidone, and mixtures thereof.

Examples of fillers suitable for use in the pharmaceutical compositions utilized herein include, but are not limited to, microcrystalline cellulose, powdered cellulose, mannitol, lactose, calcium phosphate, starch, pre-gelatinized starch, and mixtures thereof.

The binder or filler in pharmaceutical compositions is typically present in from about 50 to about 99 weight percent of the pharmaceutical composition or dosage form.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant. Disintegrants that can be used in the pharmaceutical compositions utilized herein include, but are not limited to, croscarmellose sodium, crospovidone, sodium starch glycolate, potato or tapioca starch, pre-gelatinized starch, other starches, other celluloses, gums, and mixtures thereof.

Lubricants that can be used in the pharmaceutical compositions utilized herein include, but are not limited to, calcium stearate, magnesium stearate, mineral oil, light mineral oil, glycerin, sorbitol, polyethylene glycol, other glycols, stearic acid, sodium lauryl sulfate, sodium stearyl fumarate, talc, hydrogenated vegetable oil (e.g., peanut oil, cottonseed oil, sunflower oil, sesame oil, olive oil, com oil, and soybean oil), zinc stearate, ethyl oleate, ethyl laureate, agar, and mixtures thereof. Lubricants are typically used in an amount of less than about 1 weight percent of the pharmaceutical compositions or dosage forms into which they are incorporated.

Compressed tablet formulations may optionally be film-coated to provide color, light protection, and/or taste-masking. Tablets may also be coated so as to modulate the onset, and/or rate of release in the gastrointestinal tract, so as to optimize or maximize the biological exposure of the patient to the API.

Hard capsule formulations may be produced by filling a blend or granulation of e.g., apalutamide into shells consisting of, for example, gelatin, or hypromellose. Soft gel capsule formulations may be produced.

Pharmaceutical compositions intended for oral use may be prepared from the solid dispersion formulations, and blended materials described above in accordance with the methods described herein, and other methods known to the art for the manufacture of pharmaceutical compositions. Such compositions may further contain one or more agents selected from the group consisting of sweetening agents, flavoring agents, coloring agents, and preserving agents in order to provide pharmaceutically elegant and palatable preparations.

Tablets may contain the active ingredient in admixture with non-toxic pharmaceutically acceptable excipients that are suitable for the manufacture of tablets. These excipients may be for example, inert diluents, granulating, and disintegrating agents, binding agents, glidants, lubricating agents, and antioxidants, for example, propyl gallate, butylated hydroxyanisole, and butylated hydroxy toluene. The tablets may be uncoated or they may be film coated to modify their appearance or may be coated with a functional coat to delay disintegration, and absorption in the gastrointestinal tract, and thereby provide a sustained action over a longer period.

Compositions for oral use may also be presented as capsules (e.g., hard gelatin) wherein the active ingredient is mixed with an inert solid diluent, for example, calcium carbonate, calcium phosphate or starch, or as soft gelatin capsules wherein the active ingredient is mixed with liquids or semisolids, for example, peanut oil, liquid paraffin, fractionated glycerides, surfactants or olive oil. Aqueous suspensions contain the active materials in mixture with excipients suitable for the manufacture of aqueous suspensions. Dispersible powders and granules suitable for preparation of an aqueous suspension by the addition of water provide the active ingredient in mixture with a dispersing or wetting agent, suspending agent, and one or more preservatives. In certain embodiments of the invention, the pharmaceutical compositions of the invention include a diluent system, disintegrant, salt, lubricant, glidant, and filmcoat, at concentrations of from about 3% w/w to about 58% w/w, from about 4% w/w to about 20% w/w, from about 4% w/w to about 20% w/w, from about 0.5% w/w to about 4% w/w, from about 0% w/w to about 2% w/w, and from about 1% w/w to about 5% w/w respectively, or at from about 18% w/w to about 40% w/w, from about 7% w/w to about 15% w/w, from about 7% w/w to about 18% w/w, from about1.0% w/w to about 3.0%, from about 0.1% w/w to about1.0% w/w, and from about 2.0% w/w to about 4.0% w/w, respectively. In certain embodiments, the solid dispersion formulations are blended with a diluent, one or more disintegrating agents, lubricants, and glidants. An example blended composition or oral dosage form includes mannitol, microcrystalline cellulose, croscarmellose sodium, sodium chloride, colloidal silica, sodium stearyl fumarate, and magnesium stearate.

The disintegrant may be present in a concentration from about 4% w/w to about 20% w/w or from about 7% w/w to about 15% w/w. A salt may be also present, which may be sodium chloride, potassium chloride or a combination thereof. The combination of salts and disintegrant is present at a concentration from about 5% w/w to about 35% w/w of the final pharmaceutical composition.

In certain embodiments, inactive ingredients of the core tablet are: colloidal anhydrous silica, croscarmellose sodium, hydroxypropyl methylcellulose-acetate succinate, magnesium stearate, microcrystalline cellulose, and silicified microcrystalline cellulose. In other embodiments, the tablets are finished with a film-coating consisting of the following excipients: iron oxide black, iron oxide yellow, polyethylene glycol, polyvinyl alcohol, talc, and titanium dioxide.

Methods of Dosing and Treatment Regimens

In one aspect, described herein are methods of treating non-metastatic castration-resistant prostate cancer (nmCRPC) in a human male comprising, consisting of, or consisting essentially of administering a therapeutically effective amount of an androgen-receptor inhibitor (e.g., apalutamide or enzalutamide) to a male human with a non-metastatic castration-resistant prostate cancer, wherein the androgen-receptor inhibitor is administered orally. In some embodiments, the androgen-receptor inhibitor is administered daily. In some embodiments, the androgen-receptor inhibitor is administered twice-a-day. In some embodiments, the androgen-receptor inhibitor is administered three times a day. In some embodiments, the androgen-receptor inhibitor is administered four times a day. In some embodiments, the apalutamide is administered every other day. In some embodiments, the antiandrogen is administered weekly. In some embodiments, the androgen-receptor inhibitor is administered twice a week. In some embodiments, the androgen-receptor inhibitor is administered every other week. In some embodiments, the androgen-receptor inhibitor is administered orally on a continuous daily dosage schedule.

In one embodiment, the desired dose is presented in a single dose or in divided doses administered simultaneously (or over a short period of time) or at appropriate intervals, for example as two, three, four or more sub-doses per day. In some embodiments, the androgen-receptor inhibitor is presented in divided doses that are administered simultaneously (or over a short period of time) once a day. In some embodiments, the androgen-receptor inhibitor is presented in divided doses that are administered in equal portions twice-a-day. In some embodiments, the androgen-receptor inhibitor is presented in divided doses that are administered in equal portions three times a day. In some embodiments, the androgen-receptor inhibitor is presented in divided doses that are administered in equal portions four times a day.

In certain embodiments, the androgen-receptor inhibitor is enzalutamide or apalutamide. In some embodiments, the antiandrogen is enzalutamide. In some embodiments, the androgen-receptor inhibitor is apalutamide. In some embodiments, the androgen-receptor inhibitor is darolutamide.

In general, doses of apalutamide employed for treatment of prostate cancer described herein in male humans are typically in the range of 10 mg to 1000 mg per day. In some embodiments, apalutamide is administered orally to the male human at a dose of about 30 mg per day to about 1200 mg per day. In some embodiments, apalutamide is administered orally to the male human at a dose of about 30 mg per day to about 600 mg per day. In some embodiments, apalutamide is administered orally to the male human at a dose of about 30 mg per day, about 60 mg per day, about 90 mg per day, about 120 mg per day, about 160 mg per day, about 180 mg per day, about 240 mg per day, about 300 mg per day, about 390 mg per day, about 480 mg per day, about 600 mg per day, about 780 mg per day, about 960 mg per day, or about 1200 mg per day.

In some embodiments, apalutamide is administered orally to the male human at a dose of about 240 mg per day. In some embodiments, greater than 240 mg per day of apalutamide is administered to the male human. In some embodiments, the apalutamide is administered orally to the male human at a dose of about 60 mg four times per day. In some embodiments, apalutamide is administered orally to the male human on a continuous daily dosing schedule.

In some embodiments, the enzalutamide is administered orally at a dose of about 160 mg per day. In some embodiments, greater than 160 mg per day of enzalutamide is administered.

In some embodiments, the darolutamide is administered orally at a dose of about 1200 mg per day. In some embodiments, the darolutamide is administered orally at a dose of about 600 mg, twice per day (equivalent to a total daily dose of 1200 mg). In some embodiments, greater than 1200 mg per day of darolutamide is administered.

In certain embodiments, wherein improvement in the status of the disease or condition in the human is not observed, the daily dose of androgen-receptor inhibitor is increased. In some embodiments, a once-a-day dosing schedule is changed to a twice-a-day dosing schedule. In some embodiments, a three-times a day dosing schedule is employed to increase the amount of androgen-receptor inhibitor that is administered.

In some embodiments, the amount of androgen-receptor inhibitor that is given to the human varies depending upon factors such as, but not limited to, condition and severity of the disease or condition, and the identity (e.g., weight) of the human, and the particular additional therapeutic agents that are administered (if applicable).

In certain embodiments, the dose of androgen-receptor inhibitor (antiandrogen), e.g., apalutamide, enzalutamide, or darolutamide is reduced when co-administered with one or more of:
(a) a CYP2C8 inhibitor, preferably gemfibrozil or clopidogrel; or
(b) a CYP3A4 inhibitor, preferably ketoconazole or ritonavir.

In some embodiments, the apalutamide is not co-administered with:
(a) medications that are primarily metabolized by CYP3A4, e.g., darunavir, felodipine, midazolam or simvastatin;
(b) medications that are primarily metabolized by CYP2C19, e.g., diazepam or omeprazole;
(c) medications that are primarily metabolized by CYP2C9, e.g., warfarin or phenytoin; or
(d) medications that are substrates of UGT, e.g., levothyroxine or valproic acid.

In further embodiments, the apalutamide is not co-administered with:
(a) medications that are P-gp substrates, e.g., fexofenadine, colchicine, dabigatran etexilate or digoxin; or
(b) BCRP/OATP1B1 substrates, preferably lapatinib, methotrexate, rosuvastatin, or repaglinide.

In further embodiments, a male human having said non-metastatic castration-resistant prostate cancer has received at least one prior therapy for the treatment of cancer, optionally wherein the prior therapy for the treatment of cancer is bicalutamine or flutamide. In still further embodiments, a male human having said non-metastatic castration-resistant prostate cancer is treatment naïve.

In other embodiments, a single unit dosage of a composition comprises of about 240 mg of apalutamide. In some embodiments, multiple doses of the single unit dosage composition comprising, consisting of, or consisting essentially of about 60 mg of apalutamide, e.g., 4 multiple or individual unit dosage forms, are administered to the human male. The total daily dose of apalutamide may be about 240 mg per day.

The quantity and frequency of administration will be determined by such factors as the condition of the human male, and the type and severity of the human male's disease, although appropriate dosages may be determined by clinical trials.

In one embodiment, administration may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

In one embodiment, the desired dose is presented in a single dose or in divided doses administered simultaneously (or over a short period of time) or at appropriate intervals, for example as two, three, four or more sub-doses per day. In some embodiments, the composition is presented in divided doses that are administered simultaneously (or over a short period of time) once a day. In some embodiments, the composition is presented in divided doses that are administered in equal portions twice a day. In some embodiments, the composition is presented in divided doses that are administered in equal portions three times a day. In some embodiments, the composition is presented in divided doses that are administered in equal portions four times a day.

The therapeutics may be administered in the methods of the invention by maintenance therapy, such as, e.g., once a week for a period of 6 months or more.

In some embodiments, the human male is also administered a gonadotropin-releasing hormone (GnRH) analog, e.g., concurrently. In some embodiments the human male has had (or will have) a bilateral orchiectomy.

In some embodiments, the androgen deprivation therapy (ADT) compositions utilized by the present invention can be administered in the same dosages and/or administration times and schedules as described herein for apalutamide. Compositions utilized for ADT include, but are not limited to, luteinizing hormone-releasing hormone (LHRH) agonists (e.g., leuprolide and goserelin), LHRH antagonists (e.g., degarelix), estrogens, antiandrogens (e.g., flutamide, enzalutamide, bicalutamide, and nilutamide).

The apalutamide (APA) and the androgen deprivation therapy (ADT) can be administered simultaneously (e.g., in the same composition, or in separate compositions) or at different times, e.g., sequentially. In one embodiment, the APA can be administered before administration of the ADT. In one embodiment, the ADT can be administered before administration of the APA.

In some embodiments, the human male is also administered one or more additional therapeutic agents, e.g., a composition or compound described herein. An additional therapeutic agent can be administered with the apalutamide or the androgen deprivation therapy (ADT) simultaneously (e.g., in the same composition, or in separate compositions) or can be administered before or after administration of the APA or ADT, or both before and after administration of the APA or ADT.

In further embodiments, the therapeutics described herein may be used in a treatment regimen in combination with surgery, radiation, chemotherapy, immunosuppressive agents, such as methotrexate, cyclosporin, azathioprine, mycophenolate, and FK506, antibodies, or other immunoablative agents such as anti-CD3 antibodies or other antibody therapies, cytoxin, fludarabine, cyclosporin, FK506, rapamycin, mycophenolic acid, steroids, FR901228, cytokines, and irradiation.

In one embodiment, the therapeutics can be used in combination with other chemotherapeutic agents in the methods described herein. Example chemotherapeutic agents include, but are not limited to, an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

A non-exhaustive list of chemotherapeutic agents considered for use in combination therapies include anastrozole (Arimidex®), bicalutamide (Casodex®), bleomycin sulfate (Blenoxane®), busulfan (Myleran®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Example alkylating agents include, without limitation, nitrogen mustards, ethylenimine derivatives, alkyl sulfonates, nitrosoureas and triazenes): uracil mustard (Aminouracil Mustard®, Chlorethaminacil®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, RevimmuneTM), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), Demethyldopan®, Desmethyldopan®, triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional example alkylating agents include, without limitation, Oxaliplatin (Eloxatin®); Melphalan (also known as L-PAM, L-sarcolysin, and phenylalanine mustard, Alkeran®); Altretamine (also known as hexamethylmelamine (HMM), Hexylen®); Carmustine (BiCNU®); Bendamustine (Treanda®); Busulfan (Busulfex® and Myleran®); Carboplatin (Paraplatin®); Temozolomide (Temodar® and Temodal®); Dactinomycin (also known as actinomycin-D, Cosmegen®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexylen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Examples of immunomodulators useful herein include, but are not limited to, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

A “therapeutically effective amount” or “effective amount”, used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics that include, for example, improved well-being of the patient, reduction of a tumor burden, arrested or slowed growth of a tumor, and/or absence of metastasis of cancer cells to other locations in the body.

Delivery systems useful in the context of embodiments of the invention may include time-released, delayed release, and sustained release delivery systems such that the delivery of the drugs occurs prior to, and with sufficient time to cause, sensitization of the site to be treated. The composition can be used in conjunction with other therapeutic agents or therapies. Such systems can avoid repeated administrations of the composition, thereby increasing convenience to the human male and the physician, and may be particularly suitable for certain composition embodiments of the invention.

Many types of release delivery systems are available and known to those of ordinary skill in the art. They include polymer base systems such as poly(lactide-glycolide), copolyoxalates, polyesteramides, polyorthoesters, polycaprolactones, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; sylastic systems; peptide based systems; hydrogel release systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775; 4,667,014; 4,748,034; and 5,239,660 and (b) diffusional systems in which an active component permeates at a controlled rate from a polymer such as described in U.S. Pat. Nos. 3,854,480 and 3,832,253. In addition, pump-based hardware delivery systems can be used, some of which are adapted for implantation.

### Example Embodiments

Embodiment 1 is a method of providing improved treatment benefit of non-metastatic castration resistant prostate cancer (nmCRPC) in a human male using apalutamide (APA) and an androgen deprivation therapy (ADT) (APA+ADT), said method comprising, consisting of and/or consisting essentially of:

administering a therapeutically effective amount of APA+ADT to the human male if a biological sample obtained from the human male is determined to have:
a) a luminal-like molecular subtype of prostate cancer;
b) a genomic classifier score of greater than about 0.6;
c) an increased expression of at least one signature of Class One co-regulated signatures;
d) an increased expression of at least one signature of Class Two co-regulated signatures;
e) a decreased expression of at least one signature of Class Three co-regulated signatures;
f) an increased expression of at least one signature of Class Four co-regulated signatures; or a combination thereof.

Embodiment 2 is a method of treating non-metastatic castration resistant prostate cancer (nmCRPC) in a human male, said method comprising, consisting of and/or consisting essentially of:

administering a therapeutically effective amount of apalutamide (APA) and a therapeutically effective amount of an androgen deprivation therapy (ADT) (APA+ADT) to the human male if a biological sample obtained from the human male is determined to have:
a) a luminal-like molecular subtype of prostate cancer;
b) a genomic classifier score of greater than about 0.6;
c) an increased expression of at least one signature of Class One co-regulated signatures;
d) an increased expression of at least one signature of Class Two co-regulated signatures;
e) a decreased expression of at least one signature of Class Three co-regulated signatures;
f) an increased expression of at least one signature of Class Four co-regulated signatures; or a combination thereof.

Embodiment 3 is a method of predicting a human male having a non-metastatic castration resistant prostate cancer (nmCRPC) to have an improved benefit from administration of a therapeutically effective amount of apalutamide (APA) and a therapeutically effective amount of an androgen deprivation therapy (ADT) (APA+ADT), said method comprising, consisting of and/or consisting essentially of:
a) determining if a biological sample obtained from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof, and
b) predicting that the human male to have an improved benefit from administration of the therapeutically effective amount of APA+ADT relative to sole administration of the therapeutically effective amount of the ADT based on:
   i) a luminal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof.

Embodiment 4 is a method of improving response to treating non-metastatic castration resistant prostate cancer (nmCRPC) in a human male using a combined administration of a therapeutically effective amount of apalutamide (APA) and a therapeutically effective amount of an androgen deprivation therapy (ADT) (APA+ADT) relative to sole administration of a therapeutically effective amount of the ADT, the method comprising, consisting of and/or consisting essentially of:
a) determining if a biological sample obtained from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof, and
b) improving response to combined administration of the therapeutically effective amount of APA+ADT relative to sole administration of the therapeutically effective amount of the ADT, based on:
   i) a luminal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof.

Embodiment 5 is a method of identifying a human male predicted to have an improved treatment benefit of nmCRPC from administration of a therapeutically effective amount of APA and a therapeutically effective amount of an androgen deprivation therapy (ADT) (APA+ADT) relative to sole administration of a therapeutically effective amount of the ADT, comprising, consisting of and/or consisting essentially of:
a) determining if a biological sample obtained from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures,
   or a combination thereof, and
b) predicting that the human male to have an improved benefit from administration of the therapeutically effective amount of APA+ADT relative to sole administration of the therapeutically effective amount of the ADT based on:
   i) a luminal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof.

Embodiment 6 is a method of predicting an improvement of response of nmCRPC to combined administration of a therapeutically effective amount of apalutamide (APA) and a therapeutically effective amount of an androgen deprivation therapy (ADT) (APA+ADT) relative to sole administration of a therapeutically effective amount of the ADT in a human male, comprising, consisting of and/or consisting essentially of:
a) determining if a biological sample obtained from the human male has:
   i) a luminal-like or a basal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof, and
b) predicting an improvement of response to combined administration of the therapeutically effective amount of APA+ADT relative to sole administration of the effective amount of the ADT, based on:
   i) a luminal-like molecular subtype of prostate cancer;
   ii) a genomic classifier score of greater than about 0.6;
   iii) an increased expression level of at least one signature of the Class One, Class Two, and/or Class Four co-regulated signatures;
   iv) a decreased expression level of at least one signature of the Class Three co-regulated signatures;
   or a combination thereof.

Embodiment 7 is the method of any one of embodiments 1-6, wherein the human male has undergone a prostatectomy.

Embodiment 8 is the method of any one of embodiments 1-7, wherein the biological sample is a prostate biopsy sample or a surgical tumor sample.

Embodiment 9 is the method of any one of embodiments 1-7, wherein the biological sample is a primary prostate tumor sample.

Embodiment 10 is the method of any one of embodiments 1-9, wherein metastasis-free survival (MFS) of combined administration of APA+ADT is improved by at least about 6 months relative to sole administration of ADT alone.

Embodiment 11 is the method of any one of embodiments 1-10, wherein second progression-free survival (PFS2) of combined administration of APA+ADT is improved by at least about 6 months relative to sole administration of ADT alone.

Embodiment 12 is the method of any one of embodiments 1-11, wherein the administering is by oral administration.

Embodiment 13 is the method of any one of embodiments 1-12, wherein the biological sample is determined to have a luminal-like molecular subtype of prostate cancer.

Embodiment 14 is the method of any one of embodiments 1-13, wherein the biological sample is determined to have a genomic classifier score of greater than 0.6.

Embodiment 15 is the method of embodiment 14, wherein the genomic classifier is a 22-marker genomic classifier comprising markers selected from the group consisting of LASP1, IQGAP3, NFIB, S1PR4, THBS2, ANO7, PCDH7, MYBPC1, EPPK1, TSBP, PBX1, NUSAP1, ZWILCH, UBE2C, CAMKC2N1, RABGAP1, PCAT-32, GYATL1P4/PCAT-80, TNFRSF19 and combinations thereof.

Embodiment 16 is the method of embodiment 14 or 15, wherein the human male is determined to have a high risk of metastasis based on the genomic classifier score.

Embodiment 17 is the method of any one of embodiments 1-16, wherein the biological sample is determined to have an increased expression of at least one signature of the Class One co-regulated signatures.

Embodiment 18 is the method of embodiment 17, wherein the at least one signature of the Class One co-regulated signatures is selected from the group consisting of: agell2012_1, bibikova2007_1, bismar2006_1, bismar2017_1, cheville2008_1, cuzick2011_1, cuzick2011_lm_1, decipher_1, decipherv2_2, genomic_capras_1, genomic_gleason_grade_1, genomic_gleason_grade_2, glinsky2005_1, hallmark_mtorc1_signaling, hallmark_myc_targets_v1, hallmark_myc_targets_v2, klein2014_1, lapointe2004_1, larkin2012_1, long2014_1, nakagawa2008_1, non_organ_confined_1, normaltumor_1, pam50_luminalB, penney2011_1, penney2011_lm_1, ramaswamy2003_1, saa12007_1, saal2007_pten, sdms_1, singh2002_1, staging_epe_1, staging_1ni_1, staging_svi_1, stephenson2005_1, talantov2010_1, varambally2005_1, wu2013_1, yu2007_1, and combinations thereof.

Embodiment 19 is the method of embodiment 18, wherein the at least one signature of the Class One co-regulated signatures comprises genomic_gleason_grade_2.

Embodiment 20 is the method of any one of embodiments 1-19, wherein the biological sample is determined to have an increased expression of at least one signature of the Class Two co-regulated signatures.

Embodiment 21 is the method of embodiment 20, wherein the at least one signature of the Class Two co-regulated signatures is selected from the group consisting of: ar_related_pathway_ARv7, ar_related_pathway_glucocorticoid_receptor, aros_1, docetaxel_sens_1, ergmodel_1, glinsky2004_1, hallmark_adipogenesis, hallmark_androgen_response, hallmark_angiogenesis_Brauer2013, hallmark_angiogenesis_KeggVEGF, hallmark_angiogenesis_Liberzon2015, hallmark_angiogenesis_Masiero2013, hallmark_angiogenesis_Nolan2013, hallmark_angiogenesis_Uhlik2016, hallmark_apical_surface, hallmark_bile_acid_metabolism, hallmark_cholesterol_homeostasis, hallmark_dna_repair, hallmark_e2f_targets, hallmark_fatty_acid_metabolism, hallmark_g2m_checkpoint, hallmark_glycolysis, hallmark_hedgehog_signaling, hallmark_heme_metabolism, hallmark_mitotic_spindle, hallmark_notch_signaling, hallmark_oxidative_phosphorylation, hallmark_peroxisome, hallmark_pi3k_akt_mtor_signaling, hallmark_protein_secretion, hallmark_spermatogenesis, hallmark_unfolded_protein_response, hallmark_uv_response_dn, hallmark_xenobiotic_metabolism, immunophenoscore_1_CP, immunophenoscore_1_CTLA.4, immunophenoscore_1_IDO1, immunophenoscore_1_LAG3, immunophenoscore_1_PD.1, immunophenoscore_1_PD.L2, immunophenoscore_1_Tem.CD4, immunophenoscore_1_TIGIT, kegg_mismatch_repair, kegg_non_homologous_end_joining, kegg_nucleotide_excision_repair, long2011_1, nelson_2016_AR_1, pam50_luminalA, pca_vs_mibc_1, race_1, ragnum2015_1, and combinations thereof.

Embodiment 22 is the method of embodiment 21, wherein the at least one signature of the Class Two co-regulated signatures comprises hallmark_cholesterol_homeostasis.

Embodiment 23 is the method of any one of embodiments 1-22, wherein the biological sample is determined to have a decreased expression of at least one signature of the Class Three co-regulated signatures.

Embodiment 24 is the method of embodiment 23, wherein the at least one signature of the Class Three co-regulated signatures is selected from the group consisting of: ars_1, beltran2016_1, dasatinib_sens_1, estimate2013_2_purity, hallmark_apical_junction, hallmark_apoptosis, hallmark_coagulation, hallmark_epithelial_mesenchymal_transition, hallmark_estrogen_response_early, hallmark_estrogen_response_late, hallmark_hypoxia, hallmark_kras_signaling_dn, hallmark_myogenesis, hallmark_p53_pathway, hallmark_pancreas_beta_cells, hallmark_reactive_oxigen_species_pathway, hallmark_tgf_beta_signaling, hallmark_tnfa_signaling_via_nfkb, hallmark_uv_response_up, hallmark_wnt_beta_catenin_signaling, immunophenoscore_1_ICOS, immunophenoscore_1_MDSC, immunophenoscore_1_PD.L1, immunophenoscore_1_SC, immunophenoscore_1_TIM3, immunophenoscore_1_Treg, kegg_base_excision_repair, kegg_homologous_recombination, lotan2016_1, neg_ctrl_qc, nelson2016_1, pam50_basal, portos_1, portos_2, rbloss_1, smallcell_1, smallcell_2, smallcell_3, torresroca2009_1, zhang2016_basal_1, and combinations thereof.

Embodiment 25 is the method of embodiment 24, wherein the at least one signature of the Class Three co-regulated signatures comprises beltran2016_1.

Embodiment 26 is the method of any one of embodiments 1-25, wherein the biological sample is determined to have increased expression of at least one signature of the Class Four co-regulated signatures.

Embodiment 27 is the method of embodiment 26, wherein the at least one signature of the Class Four co-regulated signatures is selected from the group consisting of: estimate2013_2 estimate, estimate2013_2 immune, estimate2013_2_stromal, hallmark_allograft_rejection, hallmark_angiogenesis, hallmark_complement, hallmark_IL2_JAK_STAT5_signaling, hallmark_IL6_JAK_STAT3_signaling, hallmark_inflammatory_response, hallmark_interferon_alpha_response, hallmark_interferon_gamma_response, hallmark_kras_signaling_up, immunophenoscore_1_Act.CD4, immunophenoscore_1_Act.CD8, immunophenoscore_1_B2M, immunophenoscore_1_CD27, immunophenoscore_1_EC, immunophenoscore_1_HLA.A, immunophenoscore_1_HLA.B, immunophenoscore_1_HLA.C, immunophenoscore_1_HLA.DPA1, immunophenoscore_1_HLA.DPB1, immunophenoscore_1_HLA.E, immunophenoscore_1_HLA.F, immunophenoscore_1_IPS, immunophenoscore_1_IPS.raw, immunophenoscore_1_MHC, immunophenoscore_1_TAP1, immunophenoscore_1_TAP2, immunophenoscore_1_Tem.CD8, and combinations thereof.

Embodiment 28 is the method of embodiment 27, wherein the at least one signature of the Class Four co-regulated signatures comprises hallmark_IL2_JAK_STAT5_signaling.

The following examples of the invention are to further illustrate the nature of the invention. It should be understood that the following examples do not limit the invention, and the scope of the invention is to be determined by the appended claims.

### EXAMPLES

nmCRPC is nonmetastatic prostate cancer that has developed resistance to androgen deprivation therapy (ADT) (Scher H I et al., J Clin Oncol. 34:1402-18 (2016)). Patients with nmCRPC with a prostate-specific antigen doubling time (PSADT) of <8-10 months are at significant risk for metastatic disease and prostate cancer-specific death, and one third of patients with nmCRPC develop bone metastatic disease within 2 years (Smith MR et al., J Clin Oncol. 31: 3800-06 (2013)). Androgen receptor inhibitors (ARIs) apalutamide (APA), enzalutamide, and darolutamide added to ongoing ADT have been shown to improve outcomes in nmCRPC (Smith MR et al., N Engl J Med. 378: 1408-18 (2018); Hussain M et al., N Engl Med. 378: 2465-74 (2018); Fizazi K et al., N Engl J Med. 380: 1235-46 (2019)). As with other ARIs, APA inhibits androgen receptor (AR) nuclear translocation, inhibits DNA binding, and impedes AR-mediated transcription (Clegg N J et al., Cancer Res. 72: 1494-1503 (2012)).

The SPARTAN clinical trial was to evaluate the efficacy and safety of apalutamide (APA) in adult men with high-risk non-metastatic castration-resistant prostate cancer (nmCRPC). See, e.g., Smith et al., N Engl J Med 378:1408-18 (2018).

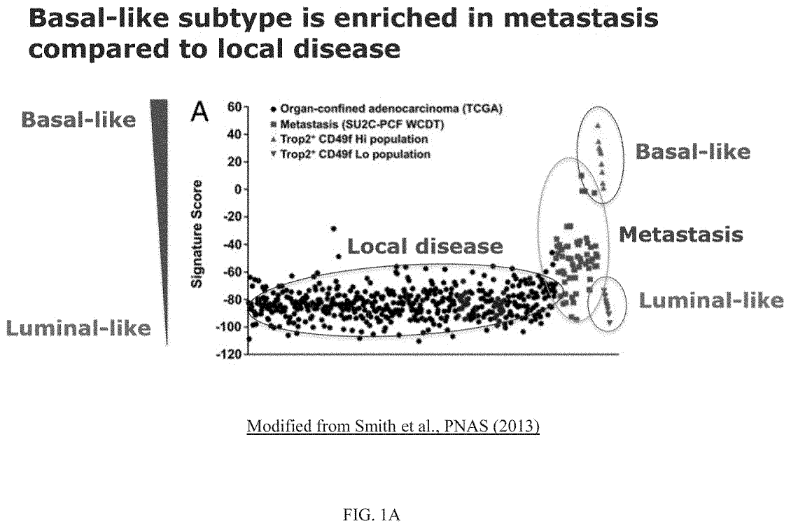

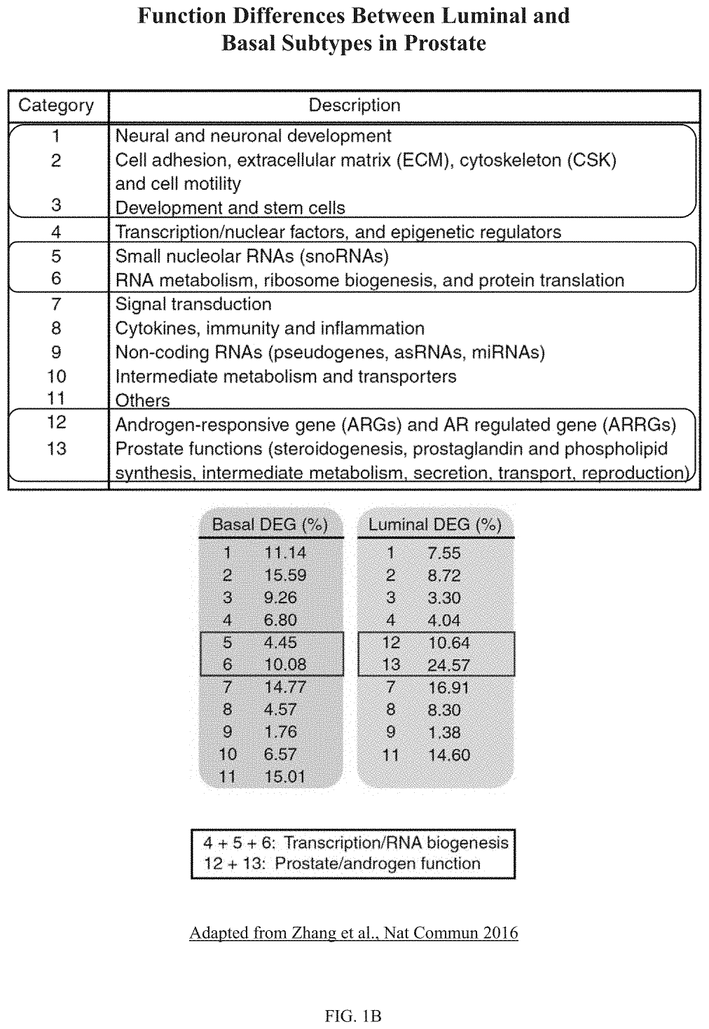

Basal and luminal subtypes represent two biologically distinct populations in prostate cancer. Both luminal and basal cells include self-sustaining lineages that can give rise to prostate cancer (Choi Net al., Cancer Cell 21(2): 253-65 (2012)). Basal-like subtypes are enriched in metastasis compared to local disease (FIG. 1A). Adult murine prostate basal and luminal cells are self-sustained lineages that can both serve as targets for prostate cancer initiation (Choi N et al., Cancer Cell 21(2): 253-65 (2012)). Basal and luminal represents two distinct phenotypes originated from different lineage dependent differentiation (Wang and Shen, Cell Rep. 8: 1339-46 (2014), See, for example, FIG. 1). Well-differentiated luminal-like cells express androgen receptors and are hormone dependent, while undifferentiated or poorly differentiated basal-like cells are more stem cell like and less hormone sensitive (Wang and Shen, Cell Rep. 8: 1339-46 (2014)). FIG. 1B shows functional differences between luminal and basal subtypes in the prostate.

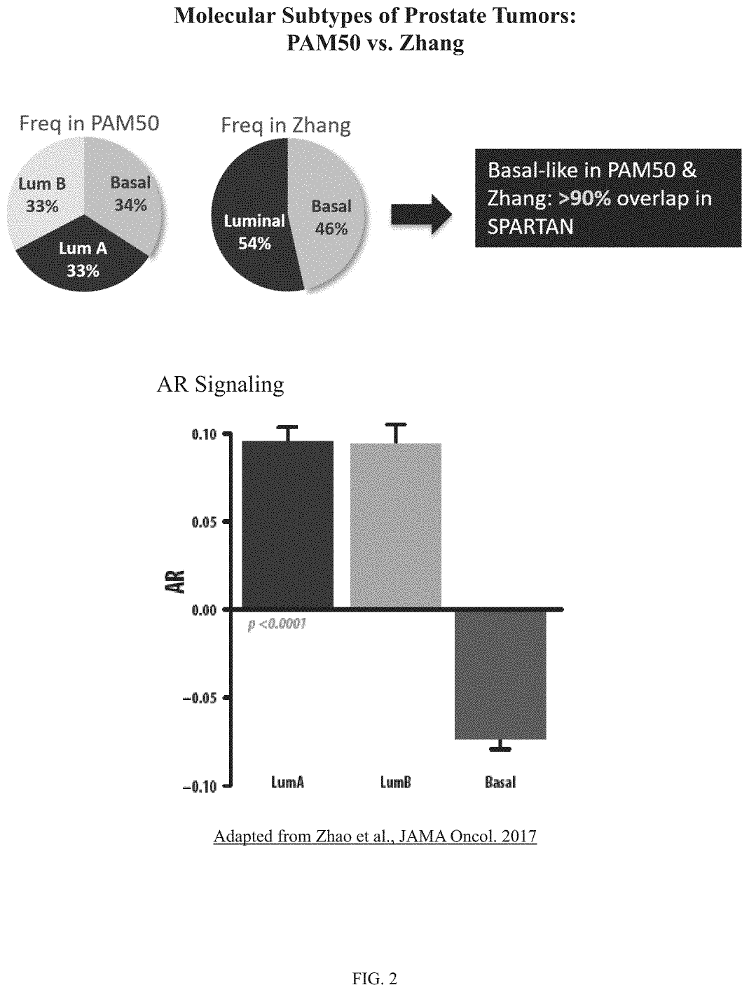

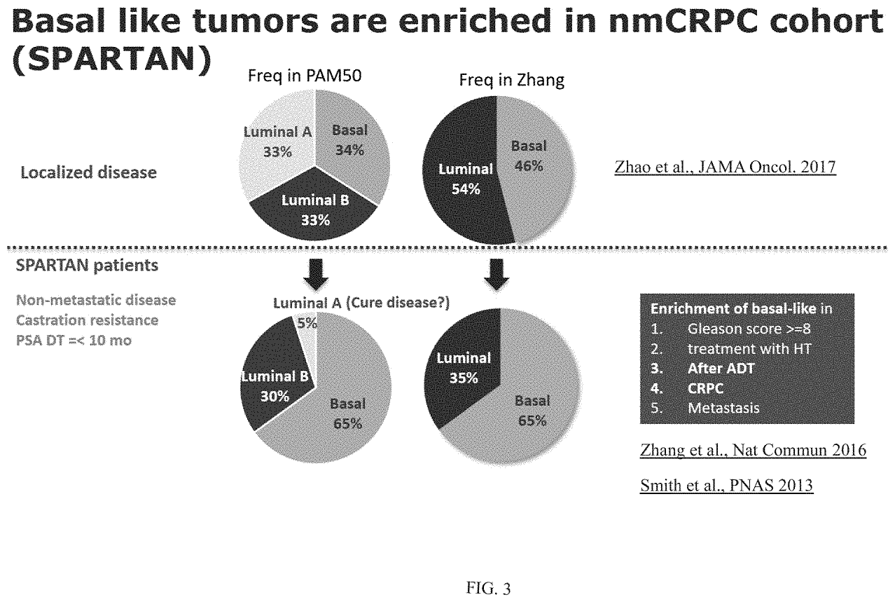

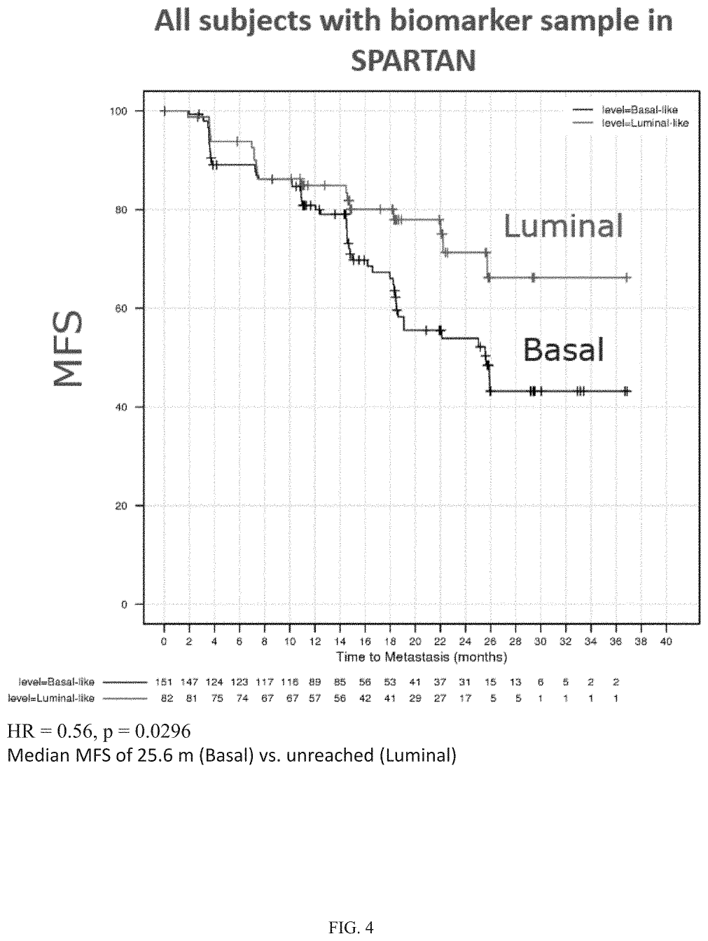

As shown in FIGS. 2 and 3, the frequency of basal-like molecular subtype reported in a PAM50 PROSIGNA® Breast Cancer Prognostic Gene Signature Assay (Guiu et al., Ann Oncol 23(12): 2997-3006 (2012), using the same gene signatures but a different platform from what was used for prostate cancer (DECIPHER®)) and Zhang (Zhao S G, et al. JAMA Oncol. 3: 1663-72 (2017)) had a greater than 90% overlap in the SPARTAN trial nmCRPC cohort and the basal-like tumors are enriched in the cohort. Although the gene signature is the same as PROSIGNA®'s assay, the data here is generated using DECIPHER®'s HuEx array. Luminal B tumors have a better prognosis when treated with ADT (no-ADT as control); and basal and luminal A tumors have poor prognosis when treated with ADT (no-ADT as control) (Zhao S G, et al. JAMA Oncol. 3:1663-72 (2017)). As shown in FIG. 4, in the SPARTAN cohort, luminal-like tumors have a longer time to metastasis (unreached) compared to basal-like (25.6 months). It has been shown that the basal-like and luminal A subtypes are resistant to ADT and that the basal subtypes of both PAM50 and Zhang are associated with poor clinical responses to ADT and that luminal B subtypes (PAM50) have selective sensitivity to ADT (Zhao S G, et al. JAMA Oncol. 3: 1663-72 (2017), FIGS. 4A and 4B; and Zhang et al. Nature Communications 7: 10798 (2016), FIG. 4O (Gleason score analysis)).

### Example 1: Identifying Molecular Determinants of Response to Apalutamide in Patients with nmCRPC in the SPARTAN Trial

Introduction

Patients with nonmetastatic castration-resistant prostate cancer (nmCRPC) with rapidly rising prostate-specific antigen (PSA), i.e., PSA doubling time (PDADT) of ≤10 months, are at high risk for developing distant metastases and experience poorer clinical outcomes compared with patients with longer PSADT (Smith M R, et al. J Clin Oncol. 23: 2918-25 (2005); Smith M R, et al. Cancer 117: 2077-85 (2011); Smith M R, et al. Lancet 379: 39-46 (2012)). Delaying metastases may improve outcomes and reduce the morbidity and mortality that accompanies disease progression (Small E J et al., Genitourinary Cancers Symposium, Abstract 161 (Feb. 8-10, 2018, San Francisco, Calif.); Lin J H et al., J Clin Oncol. 35(15 suppl). Abstract e16525 (2017)).

Apalutamide (APA) is a potent next-generation androgen receptor (AR) inhibitor that prevents nuclear translocation of AR and activation of AR-mediated signaling pathways (Clegg N J et al., Cancer Res. 72:1494-1503 (2012)). In the SPARTAN study, the addition of APA to androgen deprivation therapy (ADT) improved metastasis-free survival (MFS) for men with high-risk nmCRPC (Smith M R et al., N Engl J Med. 378: 1408-18 (2018)) versus placebo (PBO)+ADT (Small E J et al., Genitourinary Cancers Symposium, Abstract 161 (Feb. 8-10, 2018, San Francisco, Calif.); Smith M R et al., N Engl J Med. 378: 1408-18 (2018)).
Median MFS, a primary end point, was 40.5 months with APA+ADT versus 16.2 months with PBO+ADT (HR, 0.28; 95% CI, 0.23-0.35; p<0.0001).

The improvements with APA+ADT in SPARTAN were consistent across all secondary and exploratory end points (Small E J et al., Genitourinary Cancers Symposium: Abstract 161 (Feb. 8-10, 2018, San Francisco, Calif.); Smith M R et al., N Engl J Med. 378: 1408-18 (2018)), including delays in:
Progression-free survival (PFS) (HR, 0.29; 95% CI, 0.24-0.36; p<0.0001);
Time to symptomatic progression (HR, 0.45; 95% CI, 0.32-0.63; p<0.0001);
Second progression-free survival (PFS2) (HR, 0.49; 95% CI, 0.36-0.66; p<0.0001).

Improved MFS in SPARTAN patients was not accompanied by a loss in quality of life compared with baseline (Saad F et al., Lancet Oncol. 19: 1404-16 (2018)).

APA was the first drug approved for nmCRPC based on the primary end-point of MFS (Lawrence W T et al., J Urol. 6: 1264-72 (2018)).

Several molecular signatures have been validated for predicting metastases and disease aggressiveness in prostate cancer (Karnes R J et al., J Urol. 190: 2047-53 (2013); Zhang et al., Nat Commun. 7:10718 (2016); Zhao S G et al., JAMA Oncol. 3:1663-72 (2017)), including:
The DECIPHER® 22-marker mRNA-based genomic classifier (GC), which has been shown to predict (Karnes R J et al., J Urol. 190: 2047-53 (2013)):
   High risk of metastases (high GC score of >0.6).
   Low to moderate risk of metastases (low to average GC score of ≤0.6).
Luminal or basal subtypes, which have been shown to predict response to ADT (Zhao SG et al., JAMA Oncol. 3: 1663-72 (2017)):
   The luminal B subtype has been associated with sensitivity to ADT.
   The luminal A and basal subtypes may be less responsive to ADT.

Personalization of therapy based on tumor biology is useful to guide APA combination treatment strategies.

Objectives

Objectives of this transcriptome-wide analysis from patients with nmCRPC are to evaluate potential predictors of response or resistance to APA+ADT and to define high-risk patient populations.

Methods

SPARTAN (NCT01946204) was a multicenter, double-blind, randomized (2:1), placebo-controlled clinical trial in which 1207 patients with nmCRPC were randomized (2:1) to receive either ERLEADA° orally at a dose of 240 mg once daily (N=806) or placebo once daily (N=401). All patients in the SPARTAN trial received a concomitant gonadotropin-releasing hormone (GnRH) analog or had a bilateral orchiectomy. Patients were stratified by Prostate Specific Antigen (PSA) Doubling Time (PSADT), the use of bone-sparing agents, and locoregional disease. Patients were required to have a PSADT ≤10 months and confirmation of non-metastatic disease by blinded independent central review (BICR). PSA results were blinded and were not used for treatment discontinuation. Patients randomized to either arm discontinued treatment for radiographic disease progression confirmed by BICR, locoregional-only progression, initiation of new treatment, unacceptable toxicity, or withdrawal. The following patient demographics and baseline disease characteristics were balanced between the treatment arms. The median age was 74 years (range 48-97) and 26% of patients were 80 years of age or older. The racial distribution was 66% Caucasian, 12% Asian, and 6% Black. Seventy-seven percent (77%) of patients in both treatment arms had prior surgery or radiotherapy of the prostate. A majority of patients had a Gleason score of 7 or higher (78%). APA treatment was associated with significantly longer MFS in the SPARTAN cohort (See, for example, Smith M R et al., N Engl J Med. 378: 1408-18 (2018), FIG. 1A).

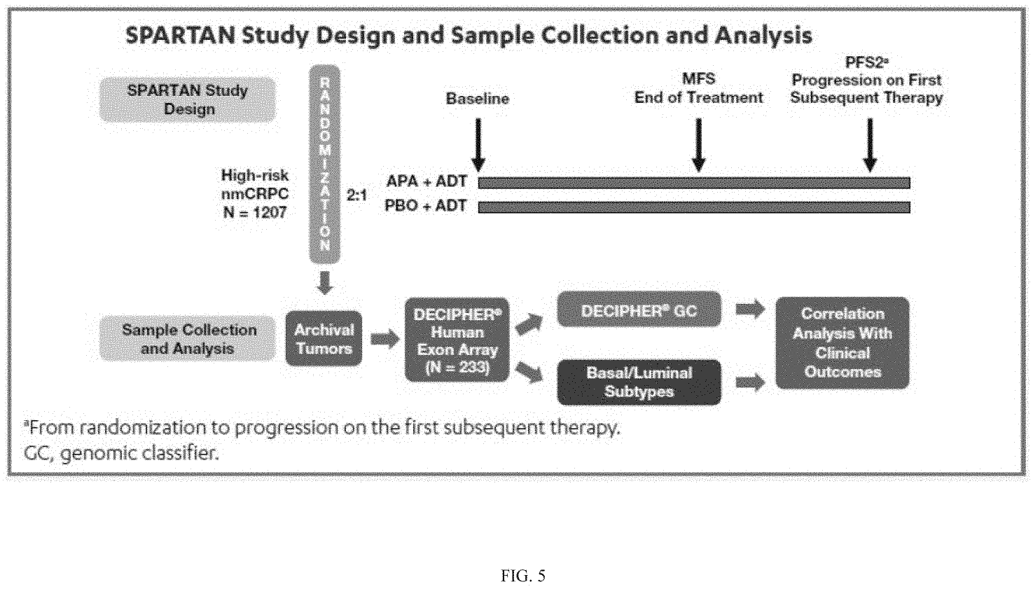

A subset of SPARTAN patients provided archival formalin-fixed paraffin-embedded tumor blocks or slides for an exploratory biomarker analysis. Of the samples, 340 were analyzed, 107 failed to meet QC acceptance criteria, and 233 were included in this analysis (biomarker population) (FIG. 5).

A DECIPHER® prostate test, a commercially available genomic assay (Decipher Biosciences, Inc., San Diego, Calif.) was performed. Analyzed samples were stratified by DECIPHER GC score and by basal-like/luminal-like subtypes.

To determine basal-like or luminal-like subtype, expression of a subset of 100 genes was assessed.
Tumors were stratified as basal-like or luminal-like based on previously defined and validated gene signatures and cutoffs (Zhang et al., Nat Commun. 7: 10718 (2016)).
Genes that were differentially expressed were identified using t test with adjusted/unadjusted p value of <0.05.
Gene expression was summarized as median centered (individual gene expression minus median) and divided by standard deviation.

The association between DECIPHER® GC scores or basal-like/luminal-like subtypes and MFS and PFS2 was assessed using a Cox proportional hazards model. Due to the lack of PFS2 events in the patient subgroup with the luminal-like subtype treated with APA+ADT, associations of PFS2 with subtypes and treatment arms were assessed using the log-rank test whenever this subgroup was involved in analyses.
MFS was defined as the time from randomization to the time of the first evidence of radiographically detectable bone or soft tissue distant metastasis or death due to any cause, whichever occurs first.
PFS2 was defined as the time from randomization to investigator-assessed disease progression on the first subsequent anticancer therapy or death of any cause prior to the start of the second subsequent anticancer therapy, whichever occurs first.

Results

Patient Population

Patients included in the SPARTAN biomarker population had aggressive disease characteristics (Table 1).

TABLE 1
Characteristics of Patients in the Biomarker Population (n = 233).

| | APO + ADT | | PBO + ADT | |
|---|---|---|---|---|
| Category | n = 154 | | n = 79 | |
| DECIPHER GC score, n (%) | | | | |
| High | 78 | (51) | 39 | (49) |
| Low to average | 76 | (49) | 40 | (51) |
| Subtype, n (%) | | | | |
| Basal | 102 | (66) | 49 | (62) |
| Luminal | 52 | (34) | 30 | (38) |
| Age, years | | | | |
| Median (range) | 73 | (49-91) | 74 | (52-90) |
| Median time from initial diagnosis to | 6.7 | | 6.6 | |
| randomization, years | | | | |
| Median PSADT, mo | 4.2 | | 4.6 | |
| ≤6 months, n (%) | 115 | (75) | 57 | (72) |
| >6 months, n (%) | 39 | (25) | 22 | (28) |
| Use of bone-sparing agent, n (%) | | | | |
| Yes | 13 | (8) | 4 | (5) |
| No | 141 | (92) | 75 | (95) |
| Local or regional nodal disease, n (%) | | | | |
| N0 | 122 | (79) | 65 | (82) |
| N1 | 32 | (21) | 14 | (18) |
| Previous prostate cancer treatment, n (%) | | | | |
| Prostatectomy or radiation therapy | 95 | (62) | 48 | (61) |
| Gonadotropin-releasing hormone analog | 151 | (98) | 78 | (99) |
| agonist | | | | |
| First-generation antiandrogen agent | 108 | (70) | 60 | (76) |

Response to APA in nmCRPC Patients with Luminal (LU)—Versus Basal (BA)—Like Tumors in the SPARTAN Trial

The SPARTAN clinical trial cohort was analyzed for the benefit to APA and ADT compared to ADT alone relative to luminal (LU)- and basal (BA)-like tumors. A total of 233 patients were assessed. Approximately 65% of patients (n=151) had the BA subtype associated with poor prognosis, indicating the high-risk nature of nmCRPC. (See, for example, Zhao S G, et al. JAMA Oncol. 3: 1663-72 (2017) FIGS. 4A and 4B; and Zhang et al. Nature Communications 7: 10798 (2016), FIG. 4O (Gleason score analysis).) Key biological pathways associated with the BA subtype in nmCRPC were neuroendocrine differentiation, epithelial-mesenchymal transition, angiogenesis, and inflammation.

Across arms, more patients in the biomarker population had the basal-like subtype (65%, n=151) than the luminal-like subtype (35%, n=82) (combined luminal A or B). Overall, 30% of patients had luminal B subtype and 5% had luminal A subtype.

The distribution of basal-like and luminal-like subtypes in SPARTAN differs from that described in a prior study of 3782 samples from patients with less aggressive localized disease who had approximately equal proportions of basal, luminal A, and luminal B subtypes classified by PAM50 (PROSIGNA® NanoString Technologies, Inc., Seattle, Wash.) (Zhao S G et al., JAMA Oncol. 3:1663-72 (2017)).

Differentially expressed genes in the basal-like and luminal (A or B) subtypes in SPARTAN are shown in FIG. 6.

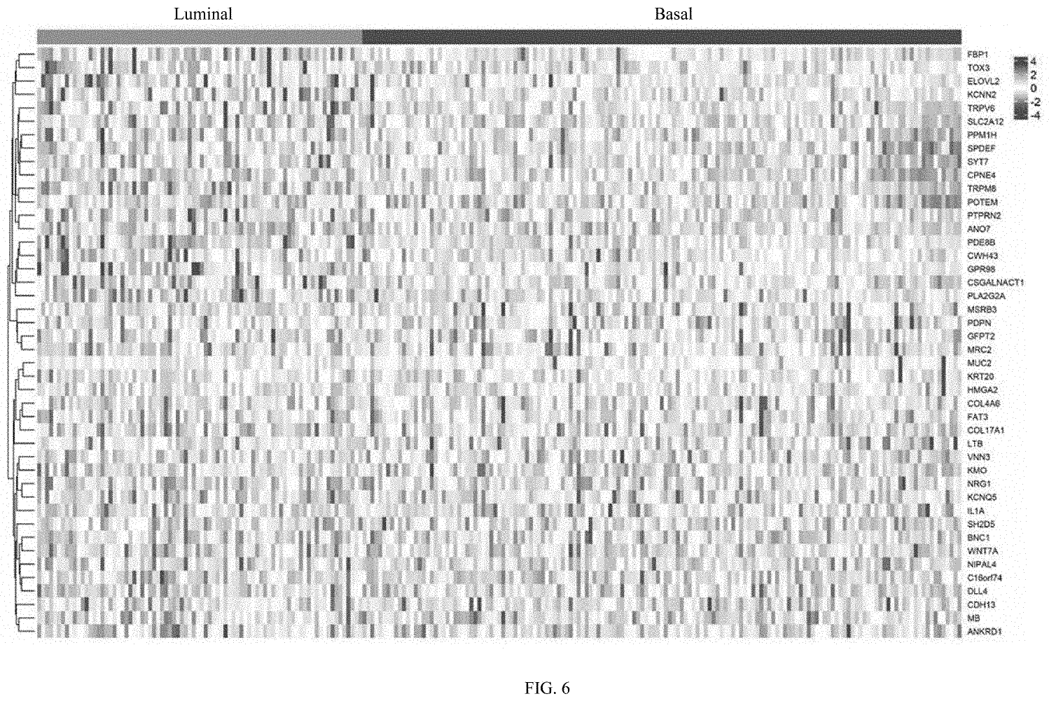

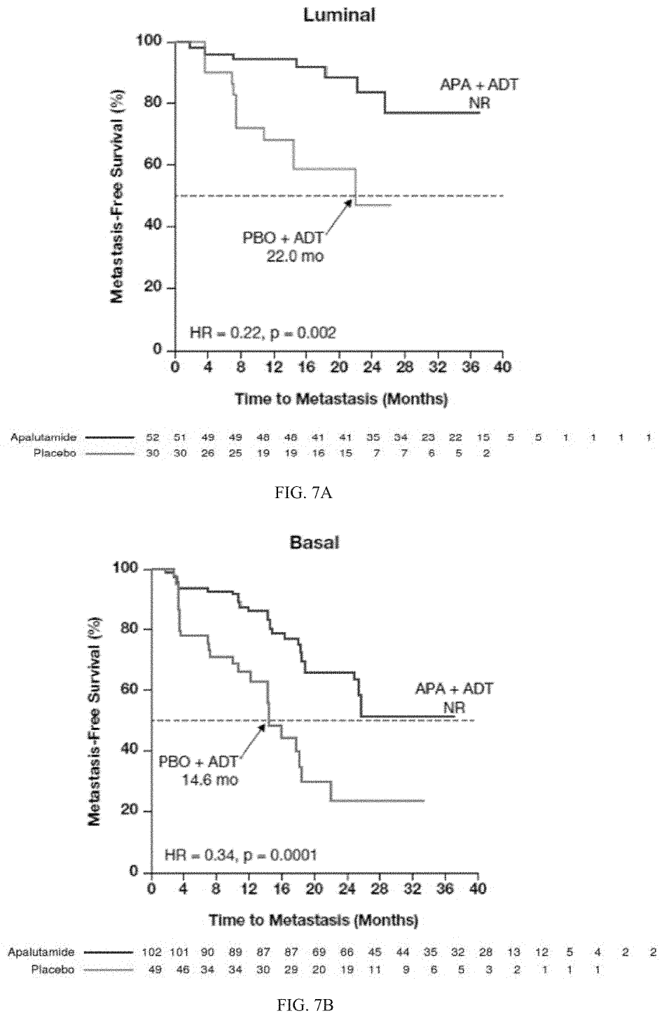

Patients with the LU subtype, known to be sensitive to ADT, and with the BA subtype, typically resistant to ADT, benefited from APA+ADT vs ADT alone: hazard ratio (HR (95CI)) for metastasis-free survival (MFS)=0.22 (0.08, 0.56), p=0.0017 and 0.34 (0.20, 0.58), p=0.0001, for LU and BA, respectively (FIGS. 7A and 7B). Patients with both basal-like and luminal-like subtypes had prolonged MFS with the addition of APA to ADT (FIGS. 7A and 7B).

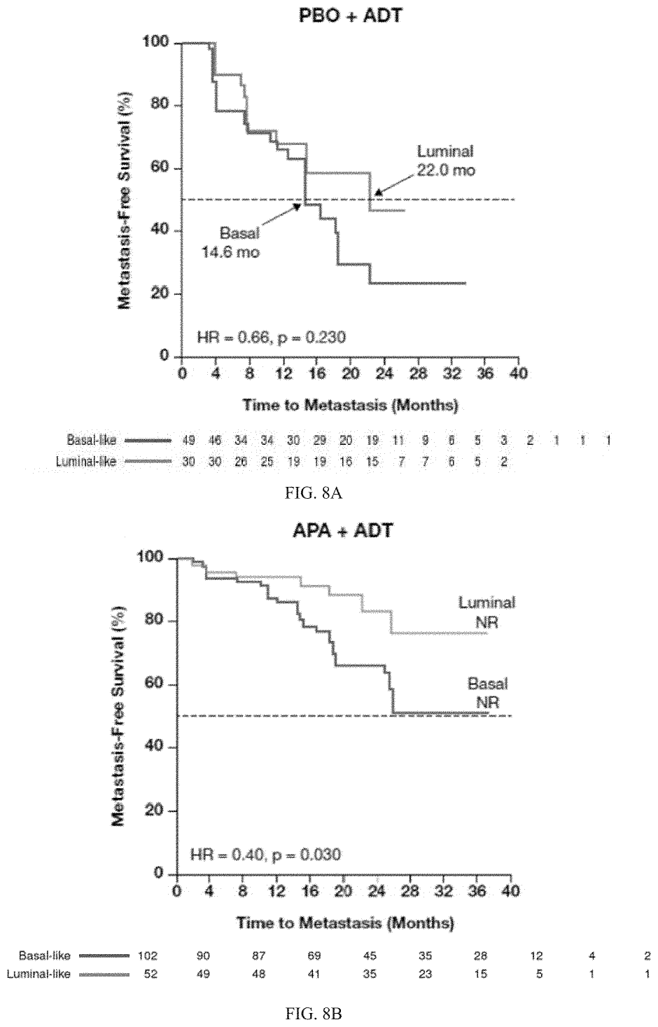

There was no difference in MFS by subtype (basal-like vs luminal-like) among patients treated with ADT alone (PBO+ADT, n=79): HR (95CI) for MFS in LU vs BA subtypes was 0.66 (0.08, 1.2), p=0.227 (FIG. 8A). Among patients treated with APA+ADT (n=154), those with the luminal-like subtype had significantly greater benefit in MFS compared with those with the basal-like subtype: HR for MFS in LU vs BA subtypes was 0.40, p=0.030 (FIG. 8B).

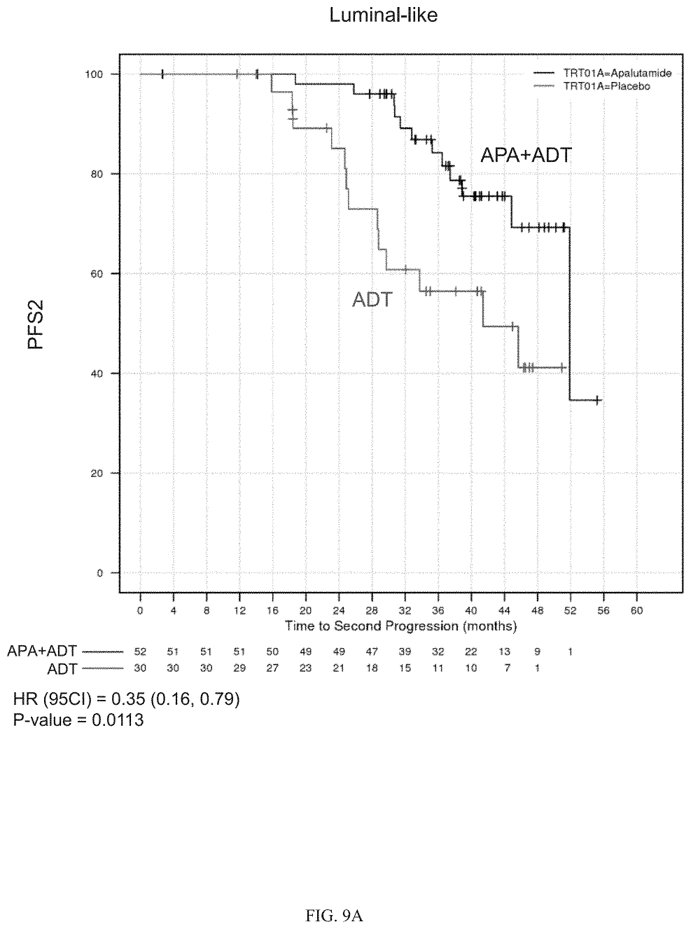

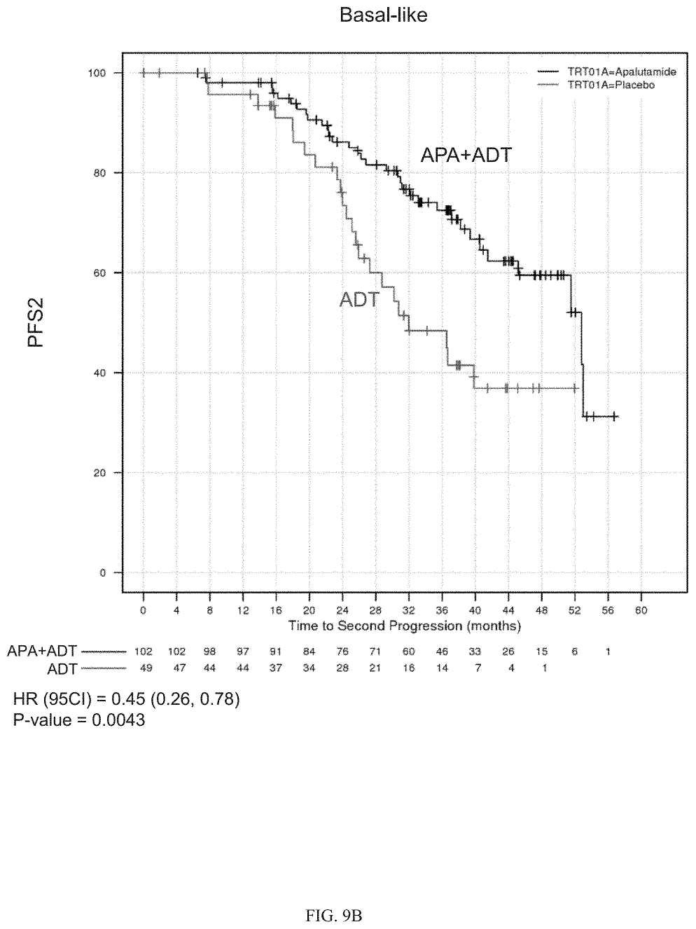

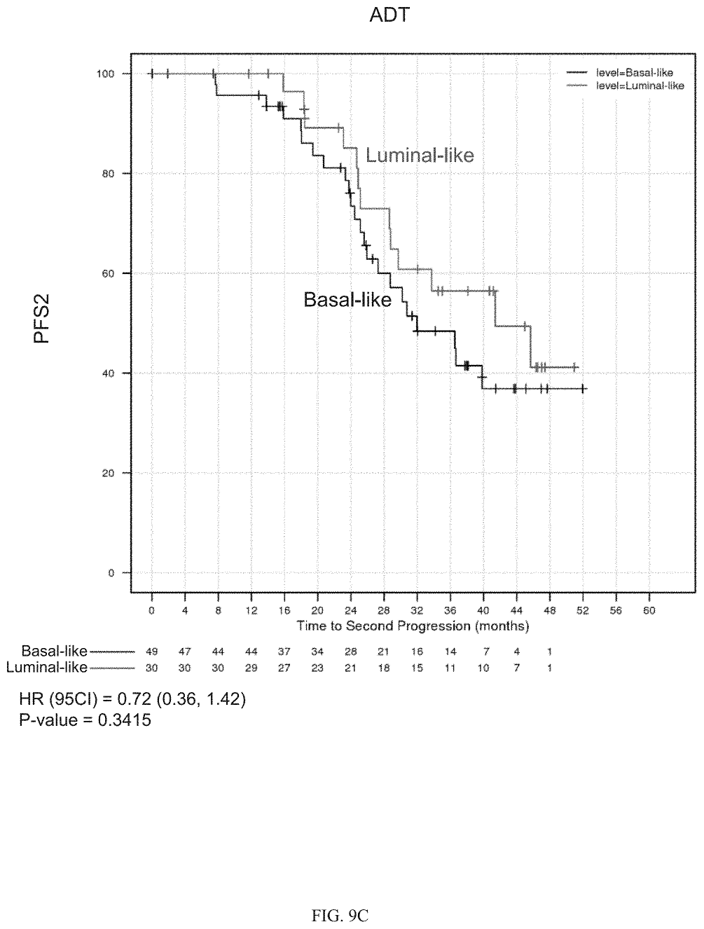

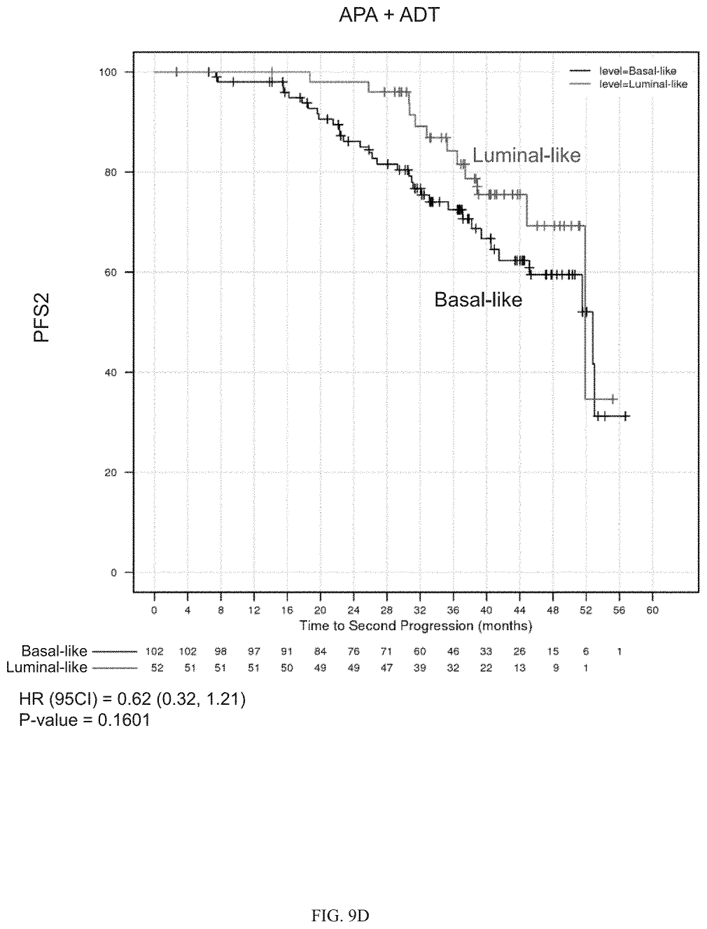

Similar benefit was observed for second progression-free survival (PFS2). Patients with the luminal-like subtype also had significantly improved PFS2 with APA+ADT versus PBO+ADT (HR (95CI), 0.35 (0.16, 0.79); p=0.0113) (FIG. 9A). Patients with the basal-like subtype had significantly improved PFS2 with APA+ADT versus PBO+ADT (HR (95CI), 0.45 (0.26, 0.78); p=0.0043) (FIG. 9B). In the ADT arm, patients with the luminal-like subtype had improved PFS2 compared with those with the basal-like subtype (HR (95CI), 0.72 (0.36, 1.42); p=0.3415) (FIG. 9C). In the APA+ADT arm, patients with the luminal-like subtype had improved PFS2 compared with those with the basal-like subtype (HR (95CI), 0.62 (0.32, 1.21); p=0.1601) (FIG. 9D).

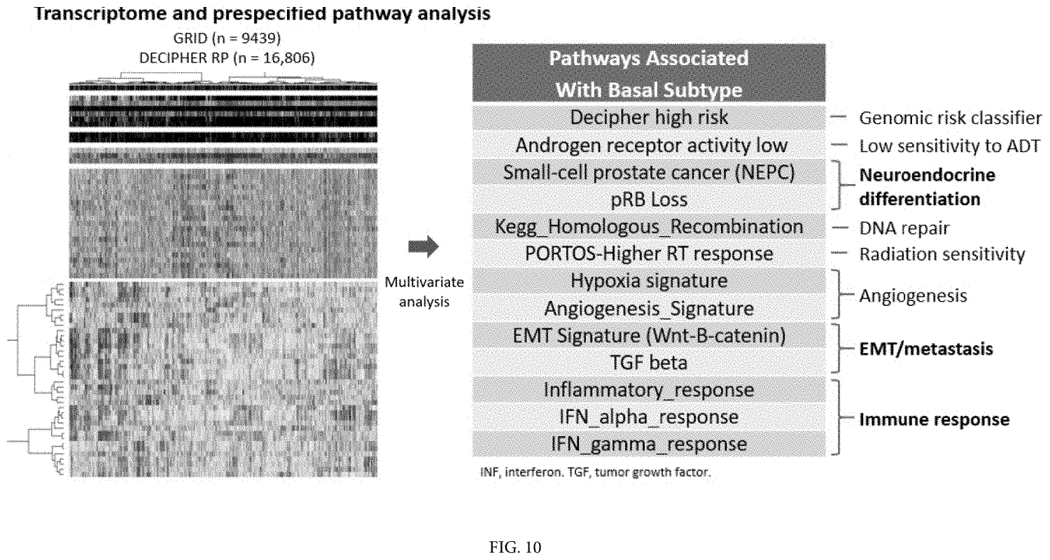

The association of pathways from Genomic Resource Information Database (GRID) with the basal-like molecular subtype was also assessed using multivariate analysis and results are shown in FIG. 10.

In summary, basal-like and luminal-like subtypes represent two biologically distinct populations in prostate cancer. Basal-like subtypes are enriched in the SPARTAN trial (65%) and have a worse prognosis when treated with ADT, while luminal-like subtypes benefit from ADT treatment. Both subtypes benefit from APA+ADT in the SPARTAN trial. Basal-like subtypes represent an ‘unmet need population’ for whom ADT is insufficient and, therefore, need APA. Further stratification allows combination strategies with APA for improved outcome. Luminal-like tumors showed sustained benefit, i.e., MFS and PFS2, to APA+ADT compared to ADT alone and basal-like tumors showed sustained benefit (MFS, PFS2) to APA+ADT compared to ADT alone. The luminal-like subtype showed maximal benefit (MFS) to APA+ADT compared to the basal-like subtype.

Response to APA in nmCRPC Patients with High-Risk and Low-to-Average Risk DECIPHER® GC in the SPARTAN Trial

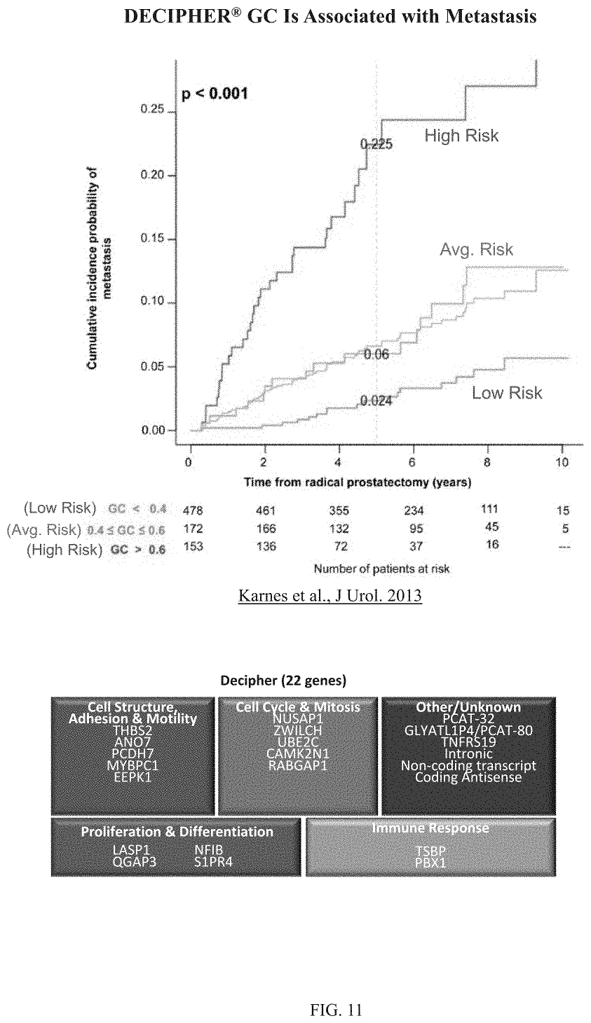

The SPARTAN study recently demonstrated that the addition of APA to ADT improved metastasis-free survival (MFS) and second progression-free survival (PFS2) in patients with nmCRPC. Transcriptome-wide profiling of available primary tumor samples from patients in SPARTAN was performed to evaluate predictors of response or resistance to APA+ADT. A commercially available genomic assay (DECIPHER® prostate test, Decipher Biosciences, Inc., San Diego, Calif.) was used to assess gene expression in archived primary tumors from SPARTAN patients. DECIPHER® GC, a 22-marker mRNA-based classifier, was validated for predicting metastatic prostate cancer (Karnes R J et al., J Urol. 190: 2047-53 (2013)) (FIG. 11), and BA/LU subtyping was validated in prostate cancer (Zhao S G, et al. JAMA Oncol. 3:1663-72 (2017); Zhang et al. Nature Communications 7: 10798 (2016)). Patients were stratified into high and low risk for developing metastases based on DECIPHER® genomic classifier (GC) score high (GC >0.6) and low to average (GC≤0.6), respectively, and into BA and LU subtypes. Gene signatures representing key biological pathways associated with the BA subtype were also assessed. The association between GC scores and subtypes and outcomes was assessed using a Cox proportional hazard model.

A total of 233 patients were assessed. Across treatment groups, proportions of high-risk and low to average risk patients in the biomarker population were similar: 50.2% (n=117) had high risk and 49.8% (n=116) had low-to-average risk. GC score subgroups were well balanced between treatment arms (Table 1).

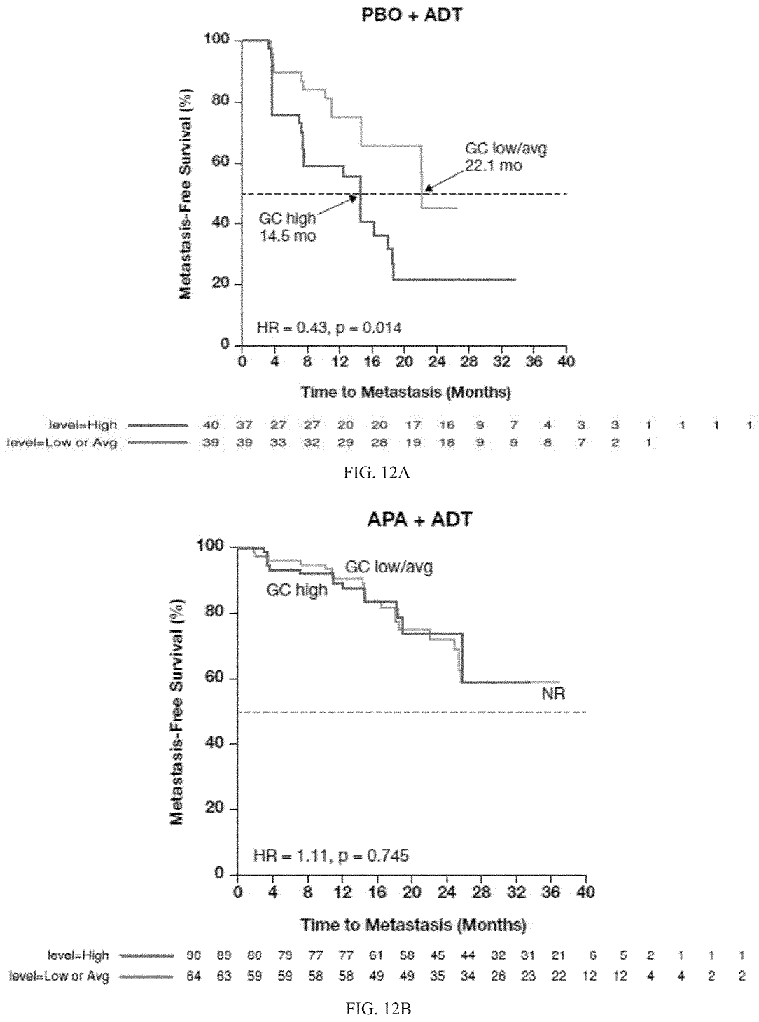

Among patients in the PBO+ADT arm, high GC scores were associated with significantly shorter time to MFS compared with low-to-average GC scores (FIG. 12A). The addition of APA to ADT led to prolonged MFS for all patients and overcame the increased risk of high GC score (FIG. 12B).

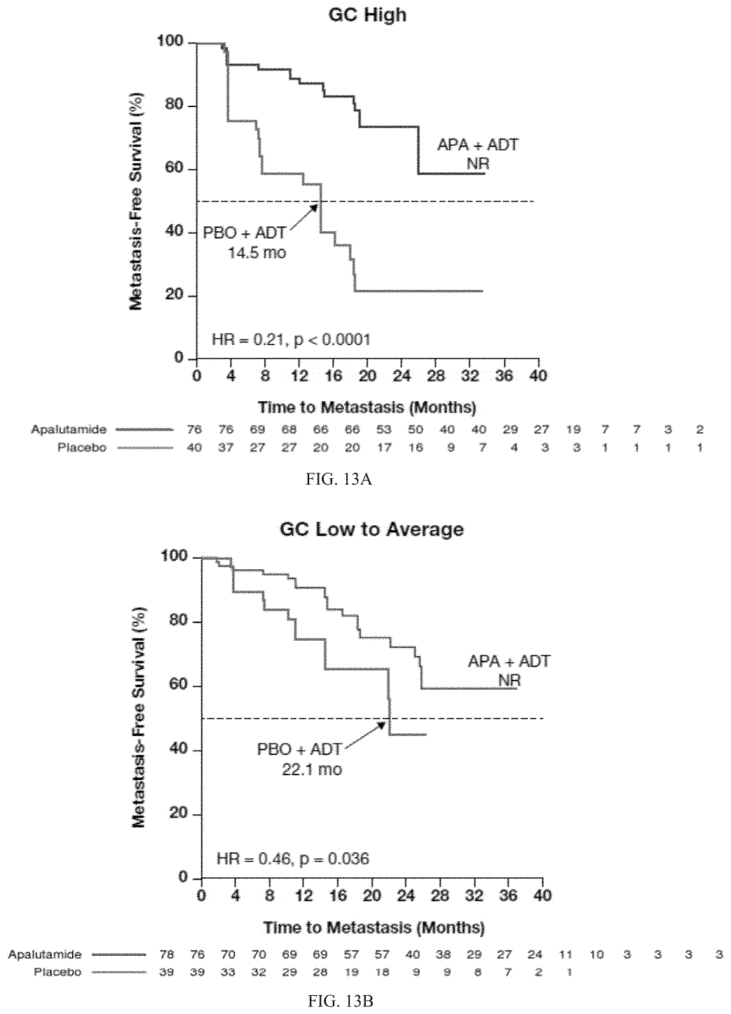

Both high and low-average DECIPHER GC score patients had improved outcomes with APA+ADT (FIGS. 13A and 13B). The magnitude of benefit in MFS was higher in patients with high DECIPHER® GC score than in those who had low to average GC scores. Poor-prognosis high GC score patients had improved MFS (HR (95CI)=0.21 (0.11, 0.40), p<0.0001) with APA+ADT vs ADT (FIG. 13A), suggesting APA overcomes the negative prognosis in these patients.

Median PFS2 in the PBO+ADT arm was 25.1 months in the high GC score subgroup versus 29.7 months in the low-to-average GC score subgroup (HR, 0.47; p=0.198). Median PFS2 in the APA+ADT arm was not reached in the high and low-to-average GC subgroups (HR, 0.29; p=0.128). Patients with high DECIPHER® GC scores had significantly longer PFS2 with APA+ADT versus PBO+ADT: Median PFS2 was not reached versus 25.1 months (HR, 0.26; p=0.008). Poor-prognosis high GC score patients had improved PFS2 (HR=0.26, p=0.0084) with APA+ADT vs ADT, suggesting APA overcomes the negative prognosis in these patients.

As evident from the clear separation in the Kaplan-Meier curves, treatment with APA+ADT improved PFS2 in patients with low to average DECIPHER GC scores (median PFS2, NR) versus PBO+ADT (median PFS2, 29.7 months) but the difference did not reach statistical significance (HR, 0.18; p=0.054) due to the small number of events in this subgroup. Poor-prognosis high GC score patients had improved MFS (HR=0.21, p<0.0001) and PFS2 (HR=0.26, p=0.0084) with APA+ADT vs ADT, suggesting APA overcomes the negative prognosis in these patients.

Conclusions

Approximately two thirds of high-risk SPARTAN patients with nmCRPC had the basal-like subtype associated with resistance to ADT, one third had the luminal B subtype, and a minority had the luminal A subtype. The majority of patients in the SPARTAN biomarker population had the basal-like subtype (65%), which is associated with aggressive disease and is typically not responsive to androgen deprivation.

Regardless of molecular subtype, all patients derived benefit from the addition of APA to ADT. The magnitude of benefit with APA+ADT was greater among patients with the luminal-like subtype than among those with the basal-like subtype. Subtyping by basal-like/luminal-like signatures may be an effective approach for patient selection in clinical studies.

Patients with both basal-like and luminal-like subtypes derived benefit from the addition of APA to ongoing ADT; however, patients with the luminal-like subtype had significantly greater benefit with APA than those with the basal-like subtype. Addition of APA to ADT overcame insensitivity to ADT in the basal-like subtype.

Half of men with nmCRPC in the SPARTAN biomarker population had a high DECIPHER® GC score, indicating aggressive disease and high risk for developing metastases. Regardless of DECIPHER® GC score, all patients had benefit from the addition of APA to ADT. The magnitude of benefit with APA+ADT was highest among patients with high DECIPHER® GC scores and greatest risk. High GC score may be useful to identify patients for early treatment intensification and for guiding APA combination treatment strategies.

Patients with high DECIPHER® GC score and basal-like subtypes have an unmet need for treatment; the results disclosed herein indicate that these patients may benefit from the addition of APA to ADT despite their high risk for progression.

Molecular signatures, such as DECIPHER® GC and BA/LU subtypes, identify patients with nmCRPC who would benefit from APA+ADT despite the high risk for developing metastasis. DECIPHER® GC is useful for identifying patients for early treatment intensification with APA or other agents, and BA/LU subtyping is an effective approach for patient selection in trials combining novel therapies with APA. DECIPHER® GC high patients represent an aggressive unmet need group in whom ADT is insufficient, urging the need to treat them with APA without delay.

Tables 2 and 3 summarize the results of the Example 1.

TABLE 2
Treatment Effects in Individual Treatment Arms of ADT
and APA + ADT Treated patients in SPARTAN study
High vs. Low-to-Average DECIPHER ® GC
scores & BA vs. LU subtypes

| | APA + ADT | ADT alone | Total |
|---|---|---|---|
| High vs low-to-average | | | |
| DECIPHER ® GC score | | | |
| Association with MFS | HR, 1.11 | HR, 0.43 | HR, 0.74 |
| | p = 0.7449 | p = 0.0144 | p = 0.1983 |
| Association with PFS2 | HR, 0.29 | HR, 0.47 | HR, 0.34 |
| | p = 0.1282 | p = 0.1976 | p = 0.0236 |
| Subtype: BA vs LU | | | |
| Association with MFS | HR, 0.40 | HR, 0.66 | HR, 0.56 |
| | p = 0.0295 | p = 0.2297 | p = 0.0296 |
| Association with PFS2 | HR, <0.001 | HR, 0.51 | HR, 0.43 |
| | p = 0.0334 | p = 0.0221 | p = 0.0951 |

TABLE 3
Treatment Effects in Biomarker Subtypes of ADT
and APA + ADT Treated patients in SPARTAN study
High vs. Low-to-Average DECIPHER ® GC
scores & BA vs. LU subtypes

ADT + APA vs ADT

| High vs low-to-average | Low-to- | |
|---|---|---|
| DECIPHER ® GC score | average risk | High risk |
| Association with MFS | HR, 0.46 | HR, 0.21 |
| | p = 0.0361 | p < 0.0001 |
| Association with PFS2 | HR, 0.18 | HR, 0.26 |
| | p = 0.0535 | p = 0.0084 |
| Subtype: BA vs LU | LU | BA |
| Association with MFS | HR, 0.22 | HR, 0.34 |
| | p = 0.0017 | p = 0.0001 |
| Association with PFS2 | HR, 0.35 | HR, 0.45 |
| | p = 0.0113 | p = 0.0043 |

### Example 2: Effects of Apalutamide (APA) to Androgen Deprivation Therapy (ADT) in Distinct Gene Expression Subclasses

Objective

One objective of this study is to characterize prostate cancer and guide novel treatment strategies, including: (1) clustering 160 pre-defined transcriptomic signatures to biologically co-regulated Classes; (2) evaluating the prognostic and predictive value of these signatures in each Class; and (3) evaluating differential treatment effect of APA+ADT based on signature expression. Another objective of this study is to define novel combination treatment strategies based on expression of signatures in all biological Classes.

Methods

SPARTAN trial data was studied. The patients were randomly assigned, in a 2:1 ratio, to receive apalutamide (240 mg per day) or placebo. All of the patients continued to receive androgen-deprivation therapy. The primary end point was metastasis-free survival, which was defined as the time from randomization to the first detection of distant metastasis on imaging or death (Smith M R et al., N Engl J Med. 378: 1408-18 (2018)).

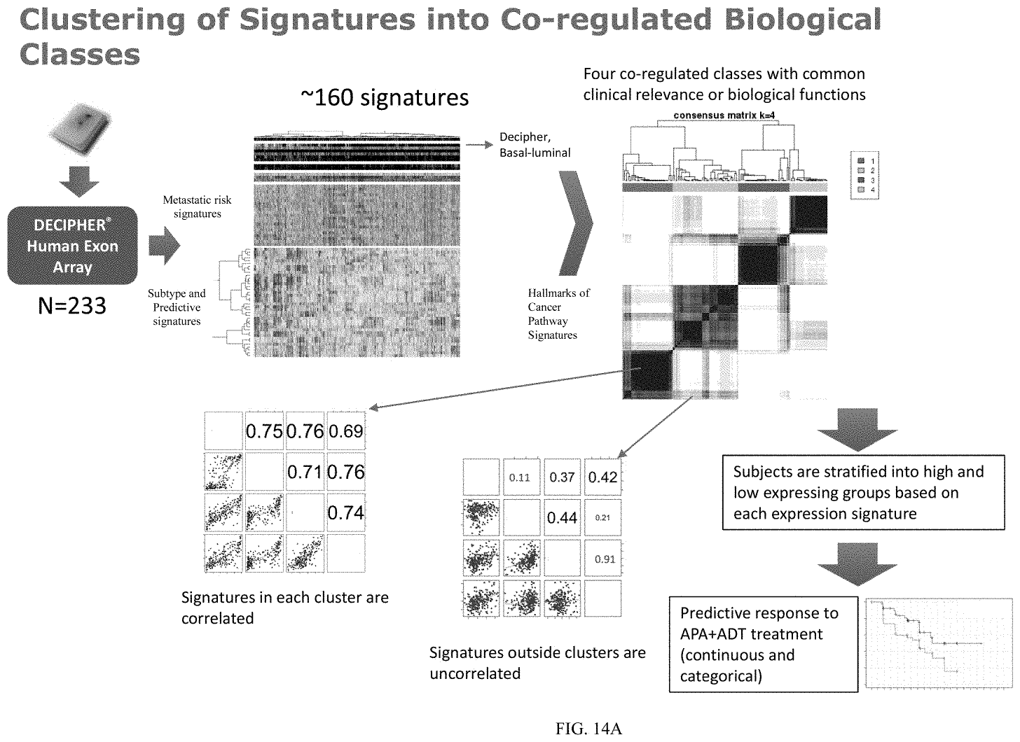

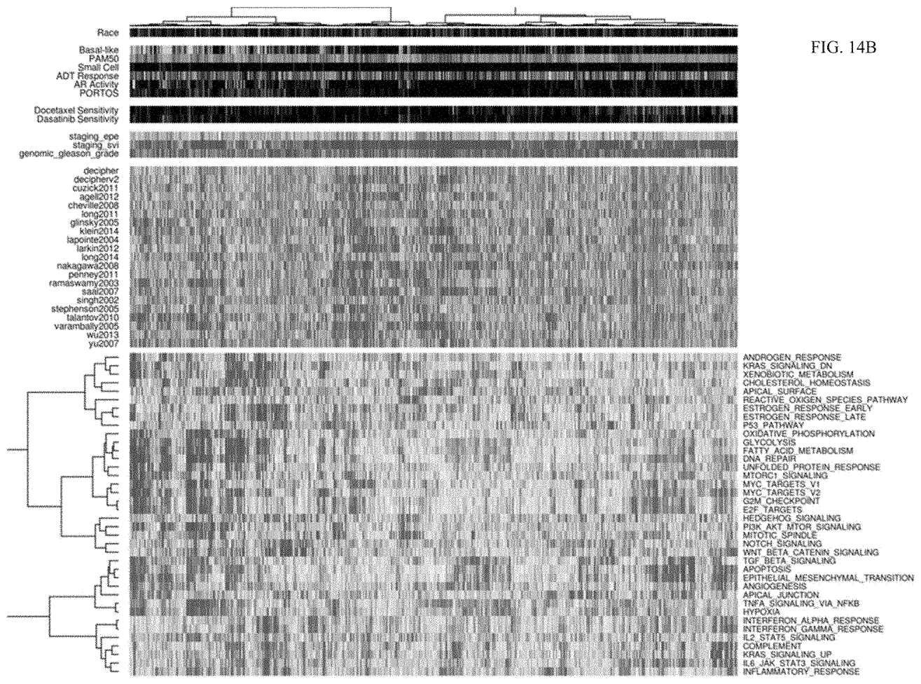

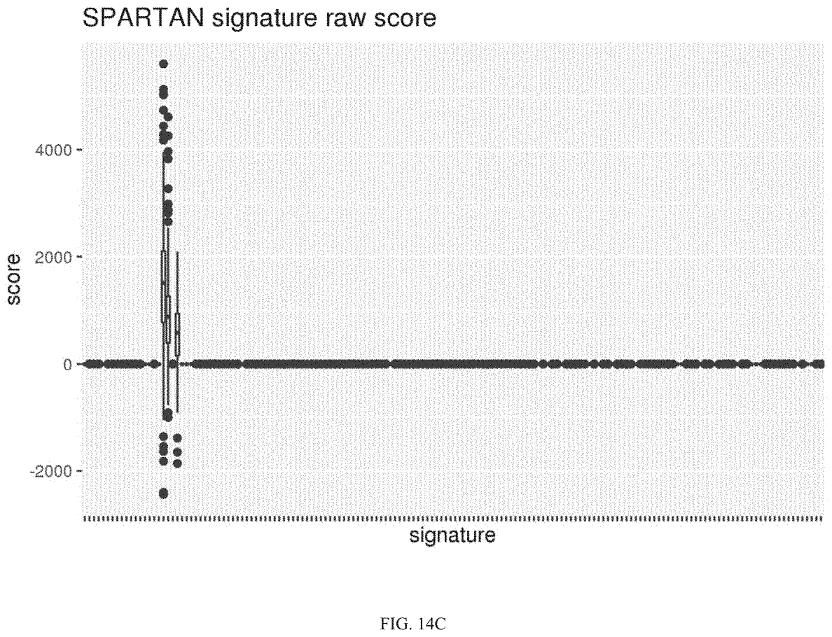

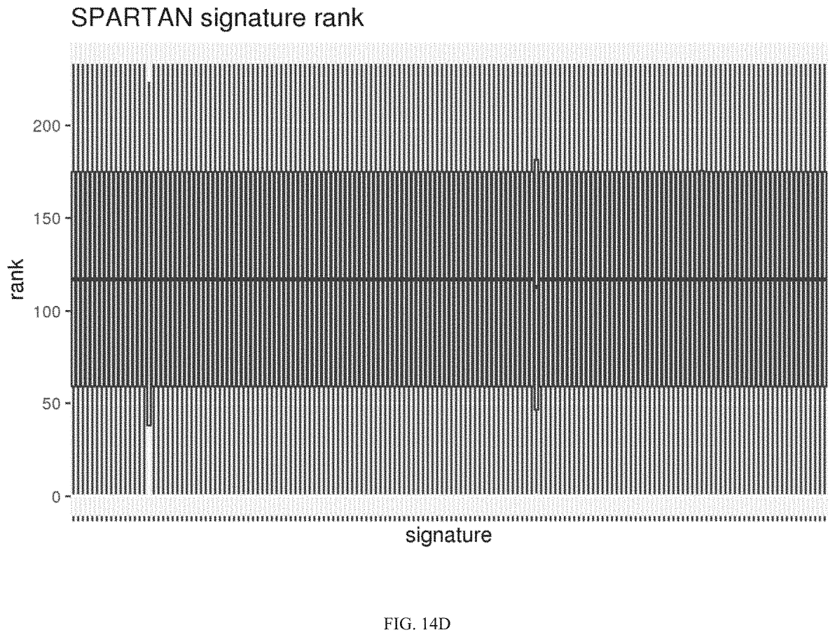

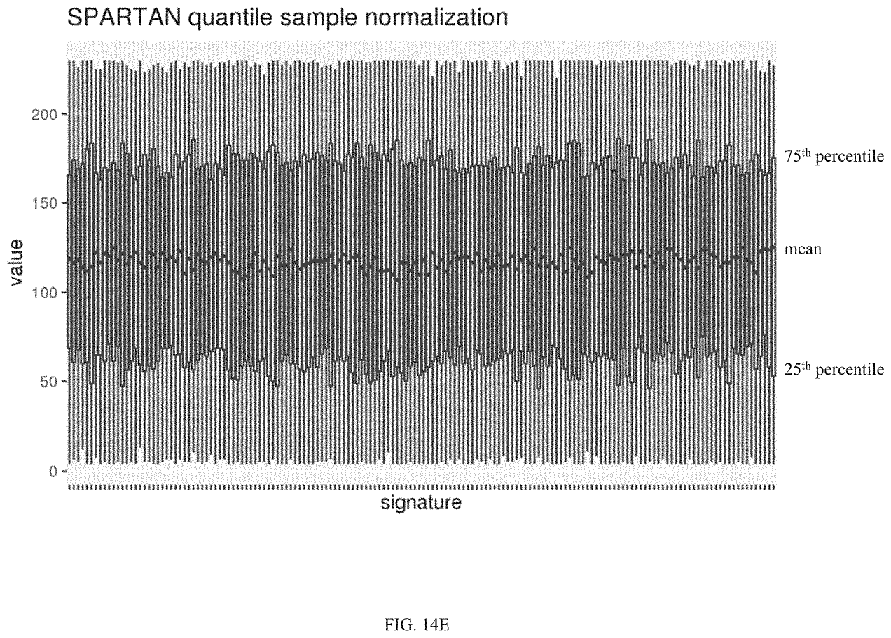

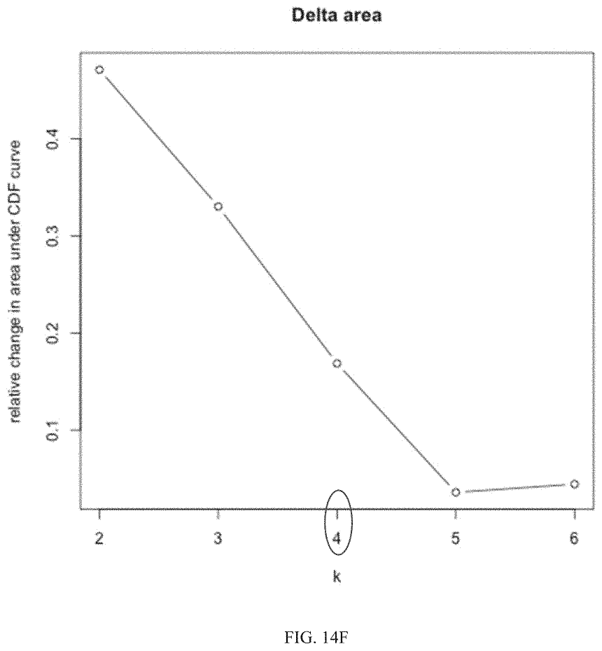

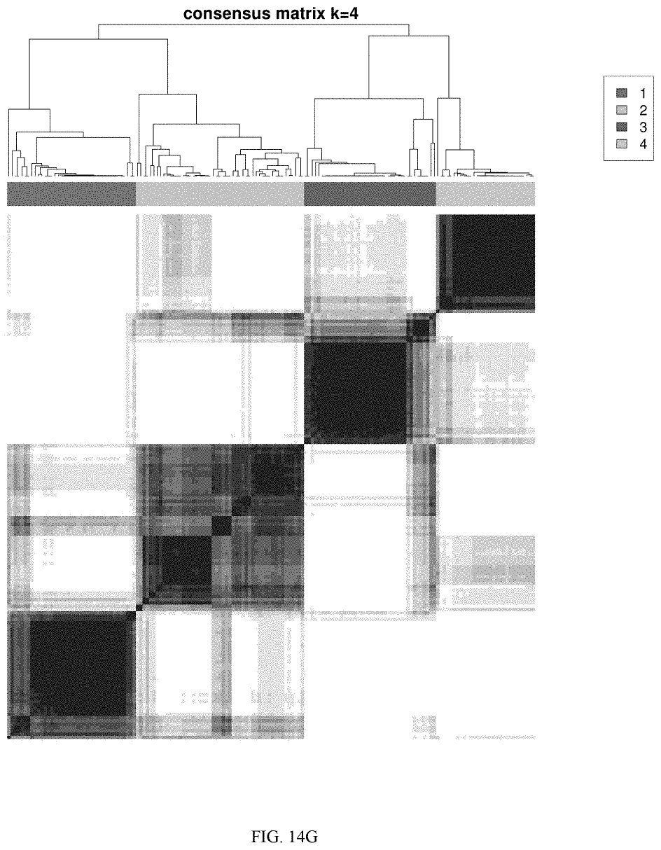

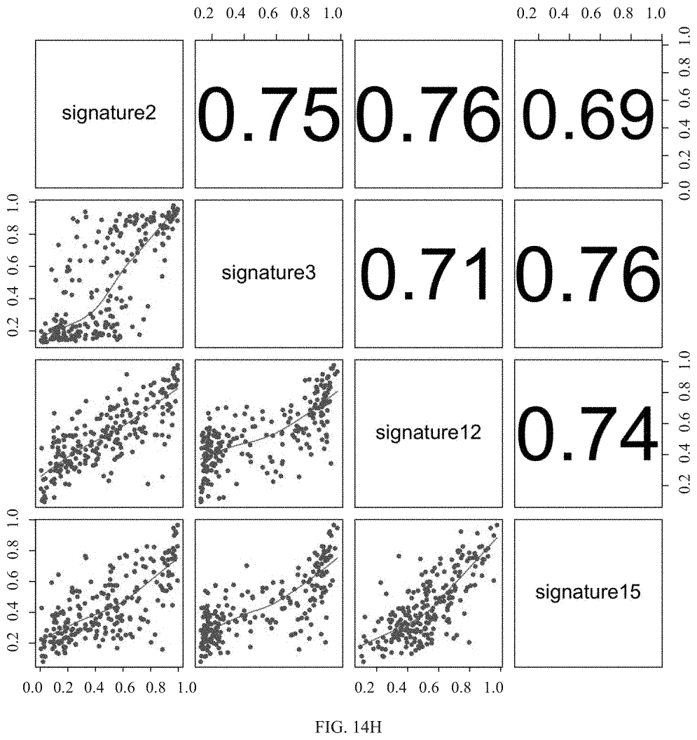

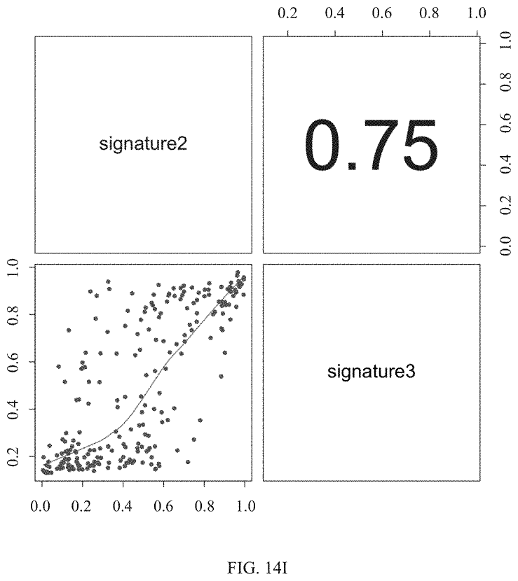

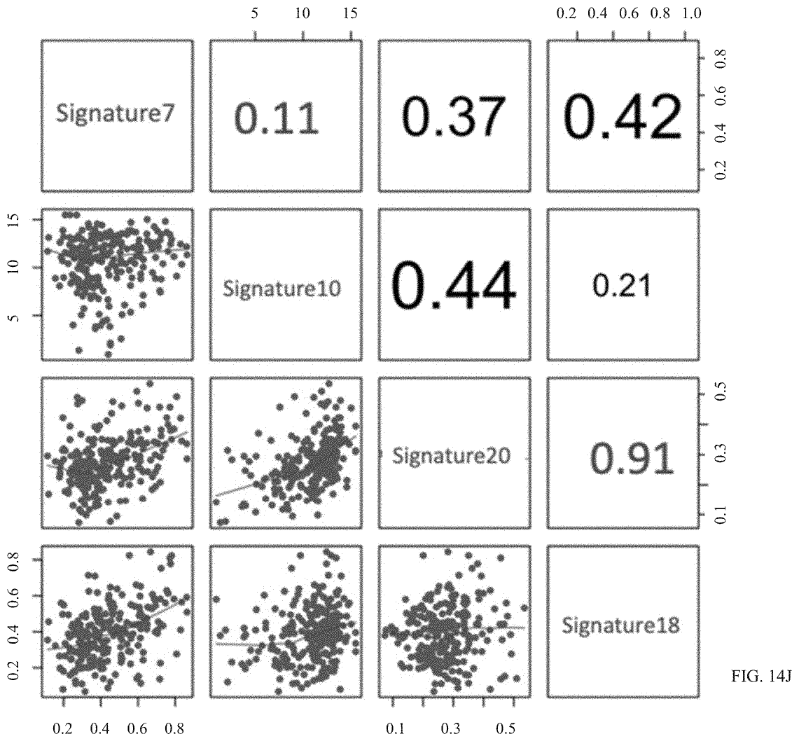

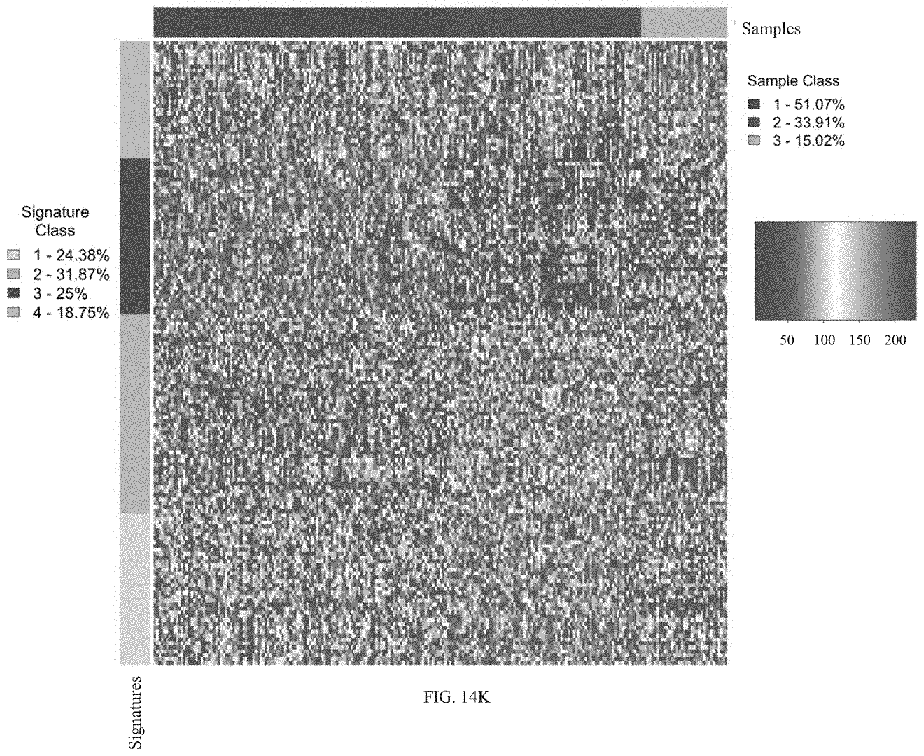

A subset of the SPARTAN patients (N=233) provided archival formalin-fixed paraffin-embedded tumor samples (blocks or slides) for an exploratory biomarker analysis (FIGS. 14A-14K). Gene expression profiles were generated by Decipher Biosciences (Decipher Biosciences, Inc., San Diego, Calif.) using the DECIPHER® Human Exon 1.0 Array platform. Data normalization was performed to identify correlations between signatures. Specifically, signatures were ranked from the lowest to the highest score. Ties were assigned by averaging tied elements, e.g., (1, 2, 3, 3, 4, 5)=(1, 2, 3.5, 3.5, 5, 6). Ranked signatures were transposed and quantile normalization was performed (FIGS. 14C-14E).

The gene expression profiles were summarized to evaluate 160 pre-defined gene expression signatures (derived from literature) indicative of clinical prognosis and prostate cancer related biology. Consensus clustering was used to identify four sets of biologically co-regulated gene expression signatures. Specifically, Classes were assigned by using the R library ConsensusClusterPlus (Wilkerson & Hayes, Bioinformatics 2010;26(12):1572-73) with the following parameters: Hclust method, 80% subsampling, 1000 iterations, average linkage, Pearson distance. The number of clusters (k=4) was selected considering the relative change in the area under the empirical cumulative distribution (FIG. 14F). The same method was used to find clusters among samples. The signature clustering and sample clustering were combined to find subset of patients that correlate with distinct signatures. The cutoff for high and low expression was defined by the median normalized expression of signatures.

The gene expression signatures were evaluated for association and interaction between expression and treatment outcome. The patients were stratified into high and low expressing groups based on each expression signature. Kaplan-Meier analysis was used to evaluate time to metastasis in high versus low expressing groups. The Cox proportional hazards model was used to investigate the association between the relative risk of metastasis and expression.

Results

Unsupervised clustering identified four classes of co-regulated signatures. Each class consists mainly of signatures with shared clinical implications and/or biological functions. The first class (C1) represents Prognosis-Related Signatures (Table 4); the second class (C2) represents Steroid Homeostasis Related Signatures (Table 5); the third class (C3) represents Hormonal Therapy Non-Responsive Basal and Neuroendocrine Like Signatures (Table 6); and the fourth class (C4) represents Immune and Stromal Signatures (Table 7). Representative signatures (RS) from each class were evaluated for association with response within each treatment arm.

Class One: Prognosis Related Signatures (24.38%)

Class One-Prognosis Related Signatures (Risk) are listed in Table 4. Representative signatures include Decipher, Luminal B, Gleason grade score, CAPRA, PSA recurrence, Aggressiveness in PCa, metastasis, PTEN loss, mtorc signaling, and PAM50-luminal B.

Between treatment groups, proportions of high- and low-expressors were similar: 50% (n=117) had high expression (median and above median) and 50% (n=116) had low expression (below median). The cutoff value was 0.49.

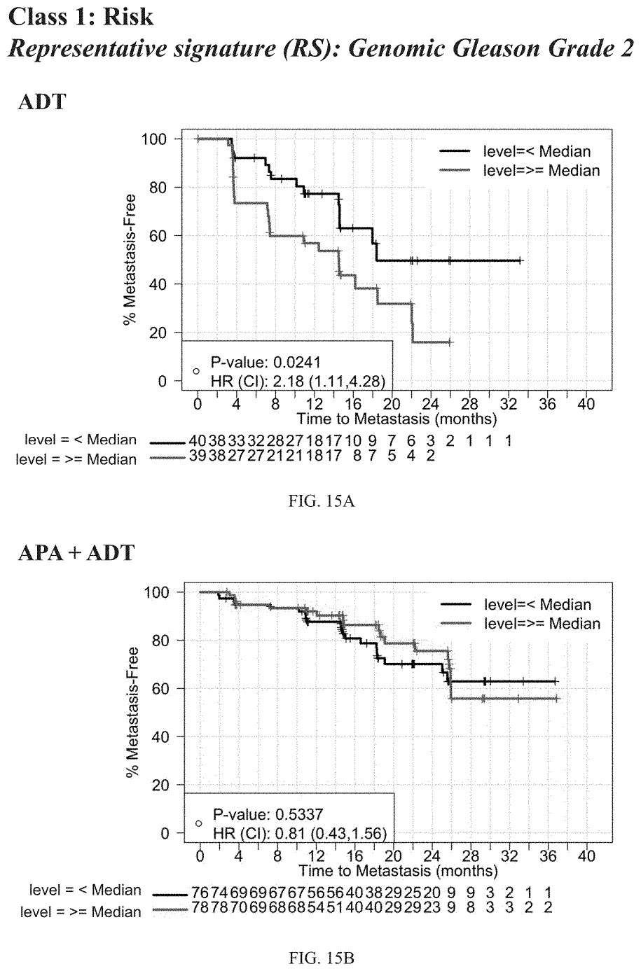

The addition of APA to ADT led to prolonged MFS for all patients and overcame the increased risk of high expression of genomic_gleason_grade_2 (a representative Class One signature). Increased expression of genomic_gleason_grade_2 predicts higher risk for metastasis (HR=2.98, p=0.002), poorer prognosis with ADT (HR: [95% CI], 2.18, 1.11-4.28, p=0.0241), and greater improved benefit with APA+ADT (HR: [95% CI], 0.81, 0.43-1.56, p=0.5337) (FIGS. 15A and 15B).

Both high- and low-expressors of genomic gleason_grade_2 had improved outcomes with APA+ADT compared to ADT. MFS are (HR: [95% CI], 0.19, 0.10-0.37, p<0.0001) and (HR: [95% CI], 0.53, 0.26-1.07, p=0.0772) for patients had high vs low expression of genomic_gleason_grade_2, respectively (FIGS. 15C and 15D), suggesting APA overcomes the negative prognosis in high-risk patients.

FIG. 15E depicts association of expression of genomic_gleason_grade_2 with relative risk by treatment arm. The relative risk in the PBO arm growths as the expression of the signature increases. The relative risk in the APA arm remains constant, even when the expression of the signature increases.

The treatment effect is (HR: [95% CI], 0.71, 0.27-1.86, p=0.4921), the effect of genomic_gleason_grade_2 is (HR: [95% CI], 2.98, 1.50-5.96, p=0.0019), and the interaction between the treatment effect and the effect of genomic_gleason_grade_2 is (HR: [95% CI] 0.36, 0.13-0.95, p=0.0390).

Class Two: Steroid Homeostasis Related Signatures (31.87%)

Class Two-Steroid Homeostasis Related Signatures (Steroid Homeostasis) are listed in Table 5. Representative signatures include Cholesterol homeostasis, Luminal A, GR activity, Docetaxel sensitivity, ARv7 activity, AR activity, ERG⁺, adipogenesis, angiogenesis, and DNA repair.

Between treatment groups, proportions of high- and low-expressors were similar: 50% (n=117) had high expression (median and above median) and 50% (n=116) had low expression (below median). The cutoff value was 0.25.

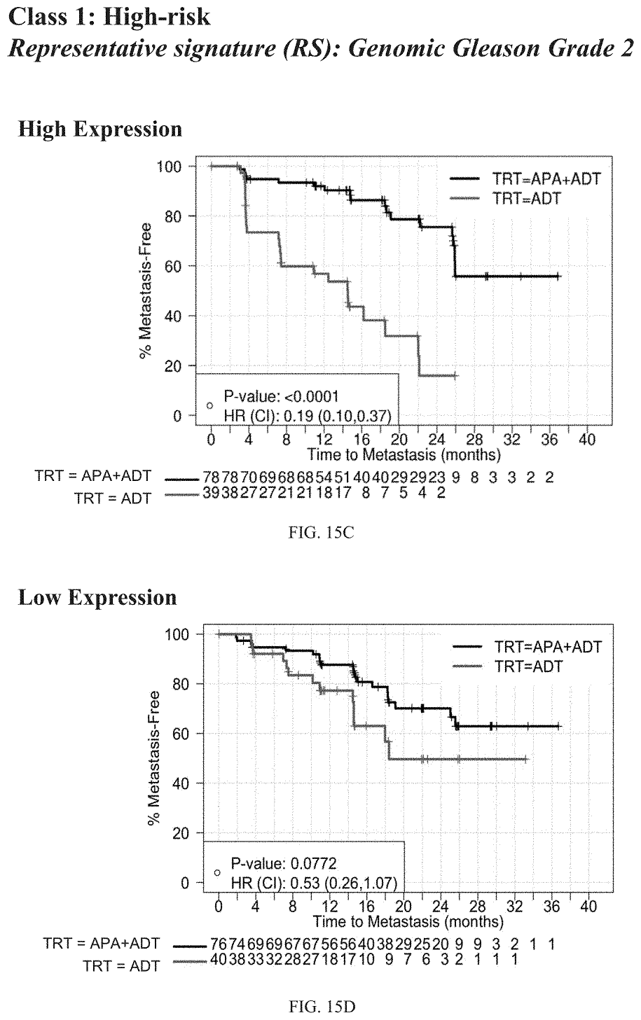

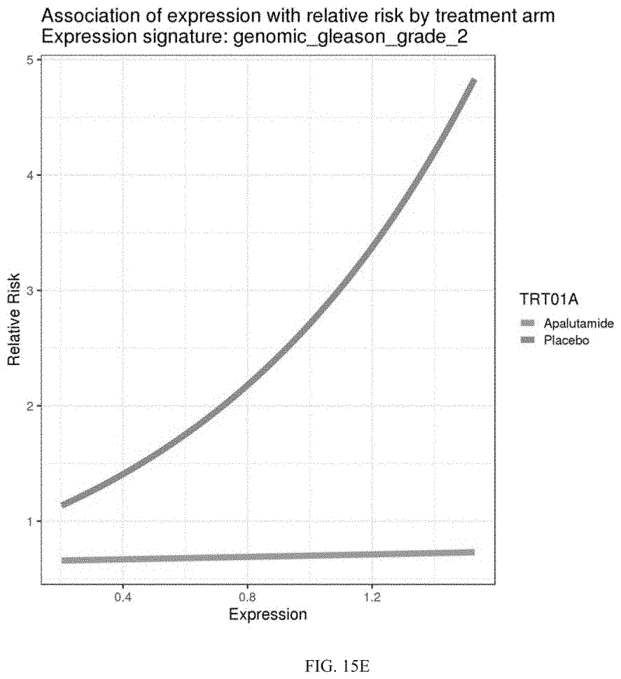

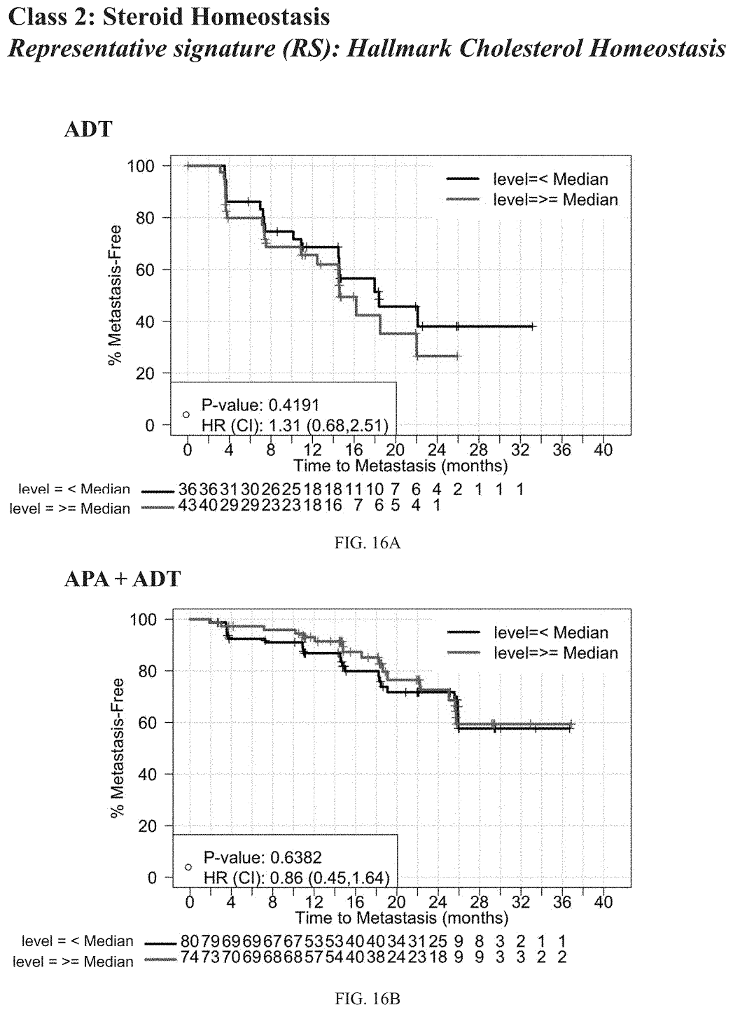

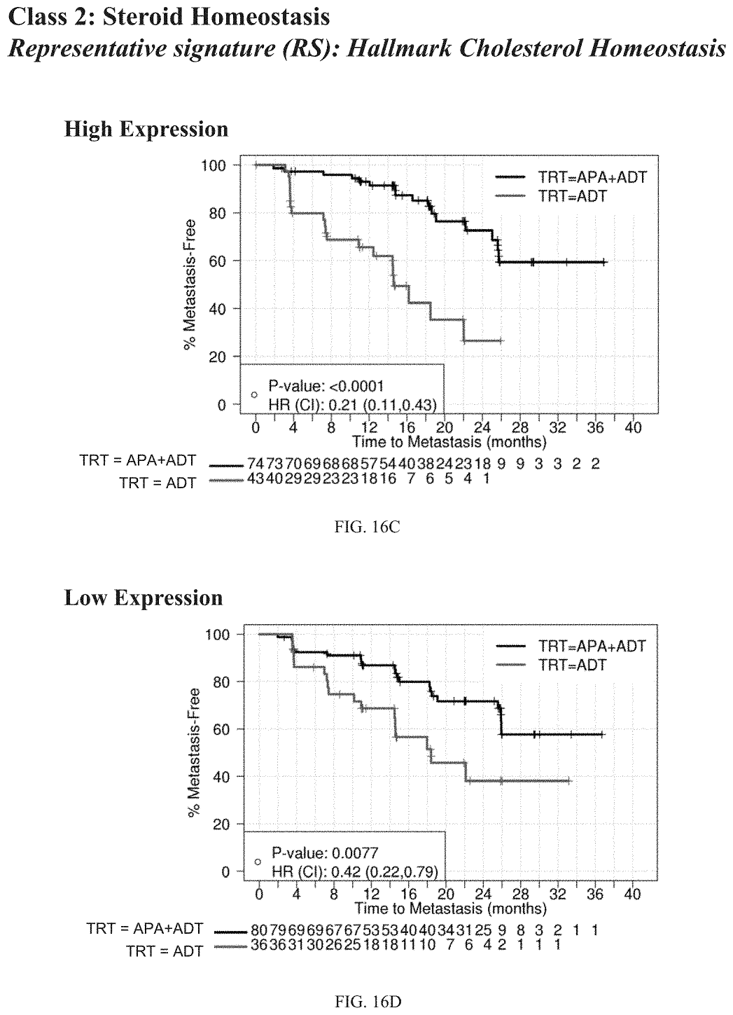

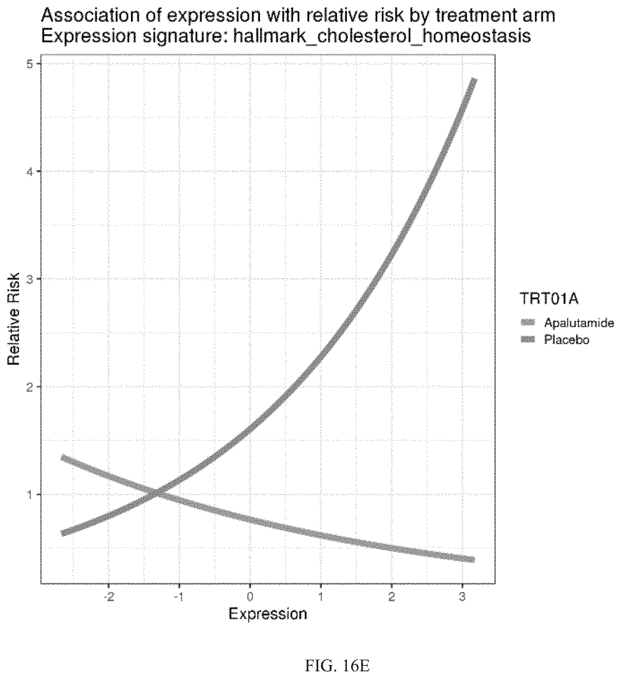

The addition of APA to ADT led to prolonged MFS for all patients and overcame the increased risk of high expression of hallmark_cholesterol_homeostasis (a representative Class Two signature). Increased expression of hallmark_cholesterol_homeostasis predicts higher risk for metastasis (HR: [95% CI] 0.57, 0.35-0.92, p=0.02), poorer prognosis with ADT (HR: [95% CI] 1.31, 0.68-2.51, p=0.4191), and greater improved benefit with APA+ADT (HR: [95% CI] 0.86, 0.45-1.64, p=0.6382) (FIGS. 16A and 16B).

Both high- and low-expressors of hallmark cholesterol homeostasis had improved outcomes with APA+ADT compared to ADT. MFS are (HR: [95% CI] 0.21, 0.11-0.43, p<0.0001) and (HR: [95% CI] 0.42, 0.22-0.79, p=0.0077) for patients had high vs low expression of Class Two Signatures, respectively (FIGS. 16C and 16D), suggesting APA overcomes the negative prognosis in high-risk patients.

FIG. 16E depicts association of expression of hallmark cholesterol homeostasis with relative risk by treatment arm. The relative risk in the PBO arm growths as the expression of the signature increases. The relative risk in the APA arm decreases with increments in the signature expression.

The treatment effect is (HR: [95% CI] 0.48, 0.26-0.88, p=0.0178), the effect of hallmark cholesterol homeostasis is (HR: [95% CI] 1.42, 1.02-1.98, p=0.0402), and the interaction between the treatment effect and the effect of hallmark cholesterol homeostasis is (HR: [95% CI] 0.57, 0.35-0.93, p=0.0232).

Class Three: Hormonal Therapy Non-Responsive Basal and Neuroendocrine Like Signatures (25%)

Class Three-Hormonal Therapy Non-Responsive Basal and Neuroendocrine Like Signatures (Neuroendocrine-Basal) are listed in Table 6. Representative signatures include RB loss status, p53 loss, PAM50-Basal, Beltran-NEPC, radiotherapy response, small cell carcinoma, Wnt-B catenin, hypoxia, and macrophage.

Between treatment groups, proportions of high- and low-expressors were similar: 50% (n=117) had high expression (median and above median) and 50% (n=116) had low expression (below median). The cutoff value was −0.44.

Approximately 27% SPARTAN biomarker tumors are molecular NE subtype (Beltran et al, Divergent clonal evolution of castration-resistant neuroendocrine prostate cancer, Nat Med. 2016; 22(3)298-305).

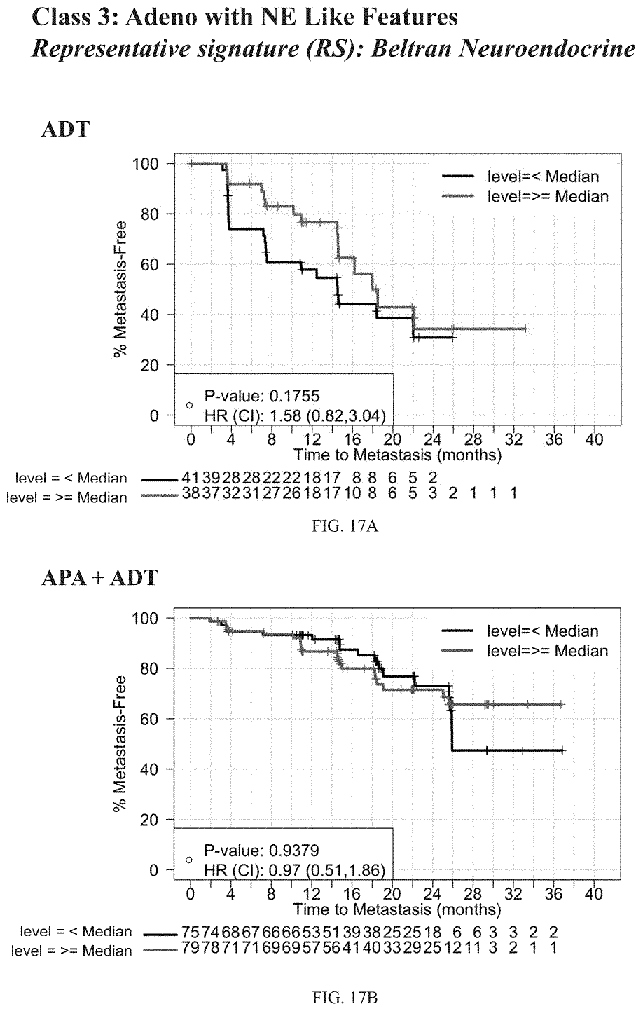

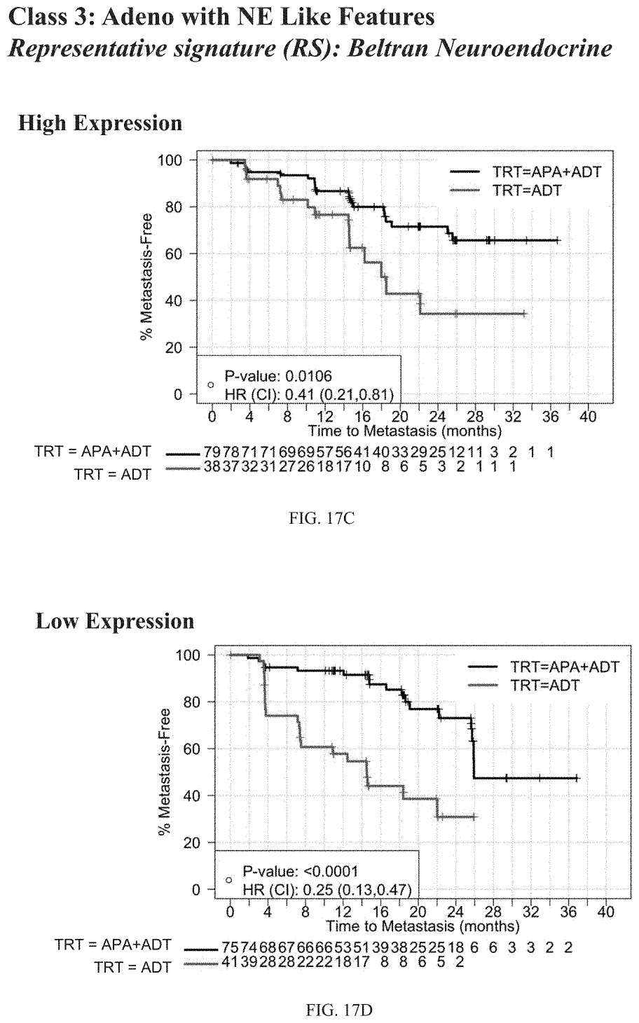

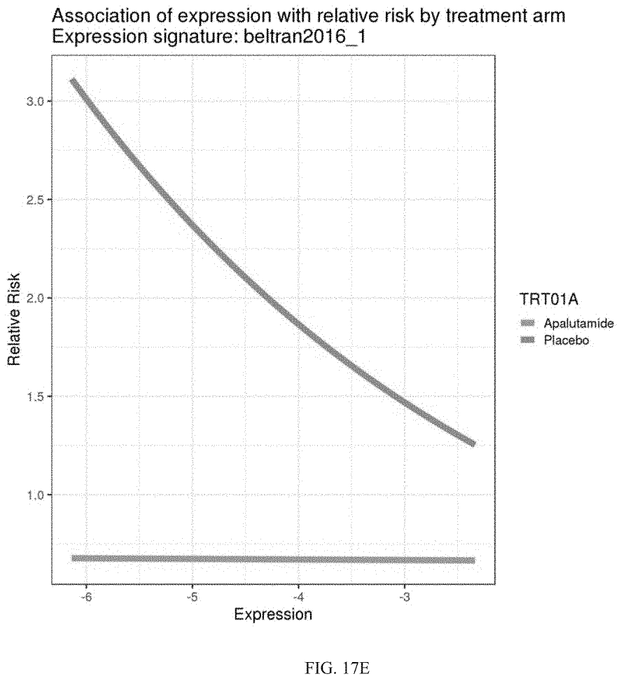

The addition of APA to ADT led to prolonged MFS for all patients. Increased expression of beltran2016_1 (a representative Class Three signature), predicts prognosis with ADT (HR: [95% CI] 1.58, 0.82-3.04, p=0.1755) and APA+ADT (HR: [95% CI] 0.97, 0.51-1.86, p=0.9379), respectively (FIGS. 17A and 17B).

Patients with high expression of beltran2016_1 (adenocarcinoma) benefit from APA+ADT (HR: [95% CI], 0.41, 0.21-0.81, p=0.0106). Low expressors of beltran2016_1 (Adeno with NE-like features) also show less risk when treated with APA+ADT (HR: [95% CI] 0.25, 0.13-0.47, p<0.0001) (FIGS. 17C and 17D).

FIG. 17E depicts association of expression of beltran2016_1 with relative risk by treatment arm. The relative risk in the PBO arm decreases as the expression of the signature increases. The relative risk in the APA arm remains constant regardless of signature expression.

The treatment effect is (HR=0.9540 (0.05, 15.65), p=0.92), the effect of beltran2016_1 is (HR=0.9854 (0.63, 1.61), p=1.00), and the interaction between the treatment effect and the effect of beltran2016_1 is (HR=0.4488 (0.69, 2.32), p=1.26).

Class Four: Immune and Stromal IL2/IL-6-JAK-STAT5 Signatures (19%)

Class Four-Immune and Stromal IL2/IL-6-JAK-STATS Signatures (Stromal/Immune) are listed in Table 7. Representative signatures include IL2-JAK-STATS signaling, IL6-JAK-STAT3 signaling, inflammatory response, Interferon γ (Ifg) response, Interferon α (Ifa) response, and allograft rejection.

Class Four signatures are stromal/immune, which means that most of the signatures in this class are related to the immune system. Hallmark gene sets won't be interchangeable with this term since hallmark related signatures are associated with different aspects of cancer biology, not only immune related.

Hallmark gene sets summarize and represent specific well-defined biological states or processes and display coherent expression. These gene sets were generated by a computational methodology based on identifying gene set overlaps and retaining genes that display coordinate expression (Liberzon A et al., The Molecular Signatures Database (MSigDB) Hallmark Gene Set Collection, Cell Syst 23;1(6):417-25 (2015)).

The original overlapping gene sets, from which a hallmark is derived is referred as its “founder” sets. The collection of 50 hallmarks condense information from over 4,000 original overlapping gene sets from v4.0 MSigDB collections Cl through C6. The hallmarks reduce noise and redundancy and provide a better delineated biological space for GSEA: see http://software.broadinstitute.org/gsea/msigdb/collection_details.jsp.

Between treatment groups, proportions of high- and low-expressors were similar: 50% (n=117) had high expression (median and above median) and 50% (n=116) had low expression (below median). The cutoff value was −0.42.

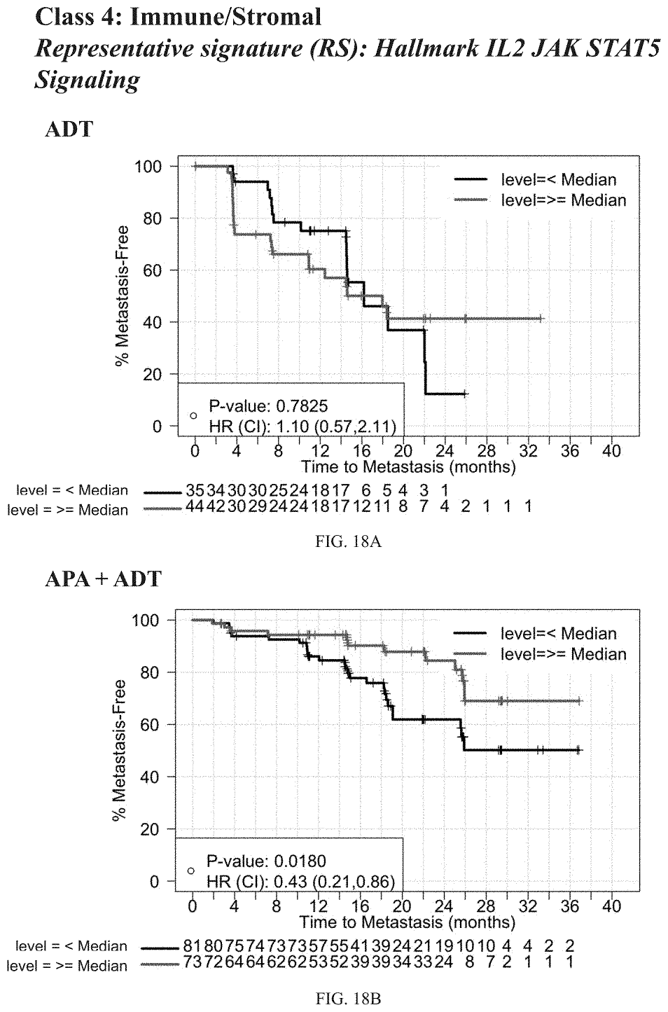

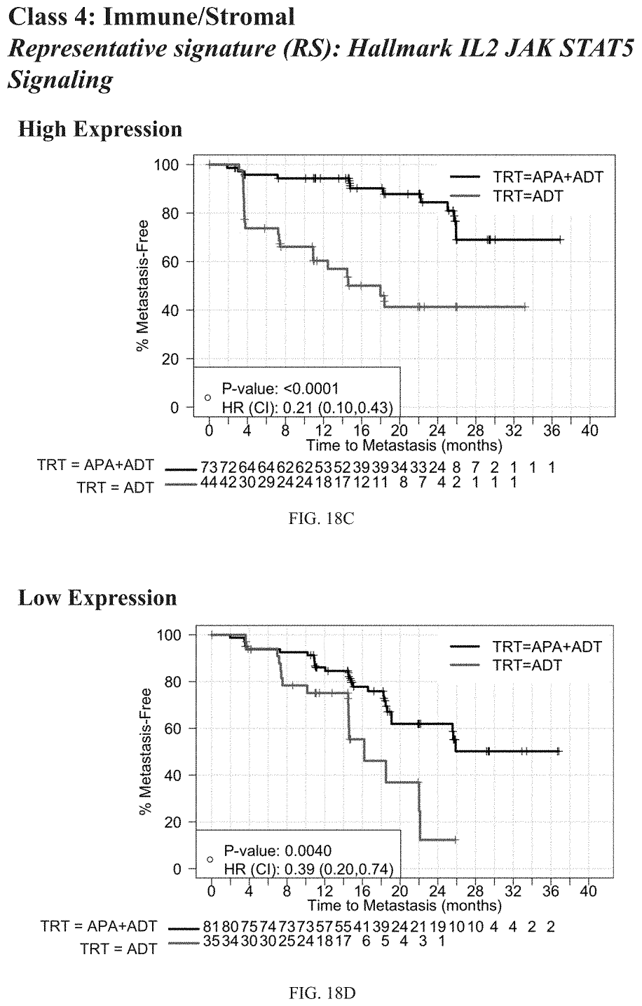

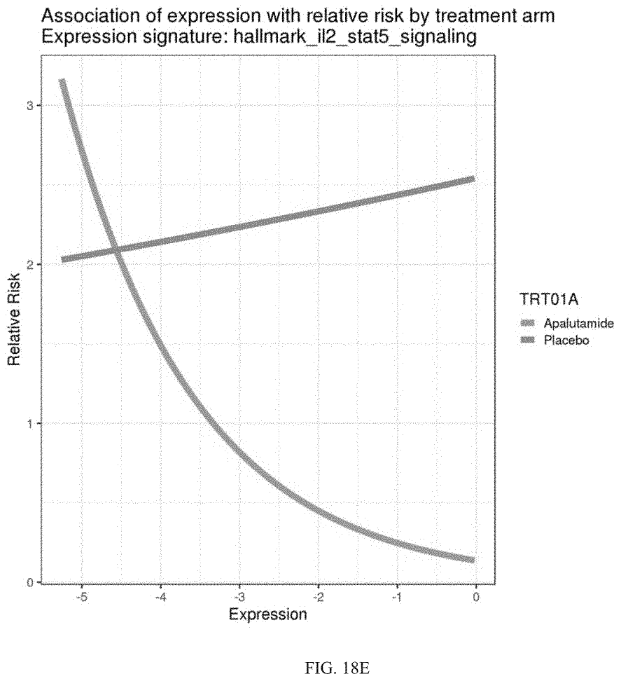

Class Four Signature expression was not associated with prognosis. However, higher expression of hallmark_IL2_JAK_STAT5_signaling (a representative Class Four signature) is associated with better outcome in APA+ADT patients (HR: [95% CI], 0.43, 0.21-0.86, p=0.0180) versus ADT patients (HR: [95% CI] 1.10, 0.57-2.11, p=0.7825) (FIGS. 18A and 18B).

Patients with low expression of hallmark_IL2_JAK_STAT5_signaling benefit from APA+ADT (HR: [95% CI] 0.39, 0.20-0.74, p=0.0040). High expressors of hallmark_IL2_JAK_STAT5_signaling also show less risk when treated with APA+ADT (HR: [95% CI] 0.21, 0.10-0.43, p<0.0001) (FIGS. 18C and 18D).

FIG. 18E depicts association of expression of hallmark_IL2_stat5_signaling with relative risk by treatment arm. The relative risk in the PBO arm growths as the expression of the signature increases. The relative risk in the APA arm rapidly decreases with increments in signature expression.

The treatment effect is (HR: [95% CI] 0.05, 0.09-0.32, p=0.0015), the effect of hallmark_IL2_JAK_STAT5_signaling is (HR: [95% CI] 0.55, 0.35-0.86, p=0.0082), and the interaction between the treatment effect and the effect of hallmark_IL2_JAK_STAT5_signaling is (HR: [95% CI] 0.53, 0.28-0.98, p=0.0444). Thus, Class Four signatures are associated with outcome dependent on APA+ADT treatment.

Conclusions

When comparing APA+ADT vs ADT, the interaction between the Class One signatures (associated with an increased risk of metastasis in placebo human males) and treatment was significantly associated with outcome. Similarly, significant signature-treatment interactions were also found in Class Two signatures. Class Three signatures were associated with higher risk of metastasis on the PBO arm, regardless of the level expression. Patients with low expression (adenocarcinoma) benefit from APA+ADT, while high expressors (Adeno with NE-like features) also show less risk when treated with APA+ADT. Finally, interaction effect between treatment and signature was also observed in Class Four stromal signatures (associated with increased risk of metastasis in higher expressor human males treated with APA+ADT).

These results further stratify clinically high-risk patients enrolled in SPARTAN based on biologically distinct classes. Consistent with observed clinical benefit, the present findings show most patients benefit from APA+ADT treatment. Moreover, the results identify subsets such as high risk, high steroidogenesis, and high stromal subtype that may benefit the most from APA+ADT treatment.

The teachings of all patents, published applications and references and other citations cited herein are incorporated by reference in their entirety.

While example embodiments have been particularly shown and described, it will be understood by those skilled in the art that various changes in form and details may be made therein without departing from the scope of the embodiments encompassed by the appended claims.

TABLE 4
Class One Co-Regulated Signatures

| Signature | Description |
|---|---|
| agell2012_1 | A 12-gene expression signature is associated with aggressive histological in prostate cancer: SEC14L1 and |
| | TCEB1 genes are potential markers of progression. |
| bibikova2007_1 | Expression signatures that correlated with Gleason score and relapse in prostate cancer |
| bismar2006_1 | Defining Aggressive Prostate Cancer Using a 12-Gene Model |
| bismar2017_1 | |
| cheville2008_1 | Gene panel model predictive of outcome in men at high-risk of systemic progression and death from prostate |
| | cancer after radical retropubic prostatectomy. |
| cuzick2011_1 | Prognostic value of an RNA expression signature derived from cell cycle proliferation genes in patients with |
| | prostate cancer: a retrospective study. |
| cuzick2011_lm_1 | Prognostic value of an RNA expression signature derived from cell cycle proliferation genes in patients with |
| | prostate cancer: a retrospective study. |
| decipher_1 | Discovery and validation of a prostate cancer genomic classifier that predicts early metastasis following radical |
| | prostatectomy. |
| decipherv2_2 | |
| genomic_capras_1 | Gleason Grade 4+ |
| genomic_gleason_grade_1 | Gleason Grade 4+ |
| genomic_gleason_grade_2 | Gleason Grade 4+ |
| glinsky2005_1 | Microarray analysis identifies a death-from-cancer signature predicting therapy failure in patients with multiple |
| | types of cancer. |
| hallmark_mtorc1_signaling | |
| hallmark_myc_targets_v1 | |
| hallmark_myc_targets_v2 | |
| klein2014_1 | A 17-gene assay to predict prostate cancer aggressiveness in the context of Gleason grade heterogeneity, tumor |
| | multifocality, and biopsy undersampling |
| lapointe2004_1 | Global transcriptome analysis of formalin-fixed prostate cancer specimens identifies biomarkers of disease |
| | recurrence. |
| larkin2012_1 | Identification of markers of prostate cancer progression using candidate gene expression |
| long2014_1 | Global transcriptome analysis of formalin-fixed prostate cancer specimens identifies biomarkers of disease |
| | recurrence |
| nakagawa2008_1 | A Tissue Biomarker Panel Predicting Systemic Progression after PSA Recurrence Post-Definitive Prostate |
| | Cancer Therapy |
| non_organ_confined_1 | Non-organ confined prostate cancer at RP |
| normaltumor_1 | tumor |
| pam50_luminalB | |
| penney2011_1 | PCSM |
| penney2011_lm_1 | |
| ramaswamy2003_1 | |
| saal2007_1 | MET |
| saal2007_pten | PTEN Loss |
| sdms_1 | MET |
| singh2002_1 | MET |
| staging_epe_1 | EPE |
| staging_lni_1 | LNI |
| staging_svi_1 | SVI |
| stephenson2005_1 | MET |
| talantov2010_1 | MET |
| varambally2005_1 | MET |
| wu2013_1 | MET |
| yu2007_1 | MET |

TABLE 5
Class Two Co-Regulated Signatures

| Signature | Description |
|---|---|
| ar_related_pathway_ARv7 | ARv7 and GR Activity |
| ar_related_pathway_glucocorticoid_receptor | ARv7 and GR Activity |
| aros_1 | Racial Variations in Prostate Cancer Molecular Subtypes and |
| | Androgen Receptor Signaling Reflect Anatomic Tumor |
| | Location |
| docetaxel_sens_1 | Docetaxel Sensitivity |
| ergmodel_1 | ERG+ |
| glinsky2004_1 | Gene expression profiling predicts clinical outcome of |
| | prostate cancer. |
| hallmark_adipogenesis | |
| hallmark_androgen_response | |
| hallmark_angiogenesis_Brauer2013 | |
| hallmark_angiogenesis_KeggVEGF | |
| hallmark_angiogenesis_Liberzon2015 | |
| hallmark_angiogenesis_Masiero2013 | |
| hallmark_angiogenesis_Nolan2013 | |
| hallmark_angiogenesis_Uhlik2016 | |
| hallmark_apical_surface | |
| hallmark_bile_acid_metabolism | |
| hallmark_cholesterol_homeostasis | |
| hallmark_dna_repair | |
| hallmark_e2f_targets | |
| hallmark_fatty_acid_metabolism | |
| hallmark_g2m_checkpoint | |
| hallmark_glycolysis | |
| hallmark_hedgehog_signaling | |
| hallmark_heme_metabolism | |
| hallmark_mitotic_spindle | |
| hallmark_notch_signaling | |
| hallmark_oxidative_phosphorylation | |
| hallmark_peroxisome | |
| hallmark_pi3k_akt_mtor_signaling | |
| hallmark_protein_secretion | |
| hallmark_spermatogenesis | |
| hallmark_unfolded_protein_response | |
| hallmark_uv_response_dn | |
| hallmark_xenobiotic_metabolism | |
| immunophenoscore_1_CP | Tumor Immunogenicity |
| immunophenoscore_1_CTLA.4 | Tumor Immunogenicity |
| immunophenoscore_1_IDO1 | Tumor Immunogenicity |
| immunophenoscore_1_LAG3 | Tumor Immunogenicity |
| immunophenoscore_1_PD.1 | Tumor Immunogenicity |
| immunophenoscore_1_PD.L2 | Tumor Immunogenicity |
| immunophenoscore_1_Tem.CD4 | Tumor Immunogenicity |
| immunophenoscore_1_TIGIT | Tumor Immunogenicity |
| kegg_mismatch_repair | |
| kegg_non_homologous_end_joining | |
| kegg_nucleotide_excision_repair | |
| long2011_1 | Protein-coding and microRNA biomarkers of recurrence of |
| | prostate cancer following radical prostatectomy |
| nelson_2016_AR_1 | AR Activity |
| pam50_luminalA | |
| pca_vs_mibc_1 | Prostate Cancer Vs Bladder Cancer |
| race_1 | Race |
| ragnum2015_1 | Pimonidazole |

TABLE 6
Class Three Co-Regulated Signatures

| Signature | Description |
|---|---|
| ars_1 | Development and validation of a prostate cancer genomic |
| | signature that predicts early ADT treatment response |
| | following radical prostatectomy |
| beltran2016_1 | Divergent clonal evolution of castration-resistant |
| | neuroendocrine prostate cancer, |
| dasatinib_sens_1 | Dasatinib Sensitivity |
| estimate2013_2_purity | Tumor, immune, and stromal content |
| hallmark_apical_junction | |
| hallmark_apoptosis | |
| hallmark_coagulation | |
| hallmark_epithelial_mesenchymal_transition | |
| hallmark_estrogen_response_early | |
| hallmark_estrogen_response_late | |
| hallmark_hypoxia | |
| hallmark_kras_signaling_dn | |
| hallmark_myogenesis | |
| hallmark_p53_pathway | |
| hallmark_pancreas_beta_cells | |
| hallmark_reactive_oxigen_species_pathway | |
| hallmark_tgf_beta_signaling | |
| hallmark_tnfa_signaling_via_nfkb | |
| hallmark_uv_response_up | |
| hallmark_wnt_beta_catenin_signaling | |
| immunophenoscore_1_ICOS | Tumor Immunogenicity |
| immunophenoscore_1_MDSC | Tumor Immunogenicity |
| immunophenoscore_1_PD.L1 | Tumor Immunogenicity |
| immunophenoscore_1_SC | Tumor Immunogenicity |
| immunophenoscore_1_TIM3 | Tumor Immunogenicity |
| immunophenoscore_1_Treg | Tumor Immunogenicity |
| kegg_base_excision_repair | |
| kegg_homologous_recombination | |
| lotan2016_1 | Neuroendocrine |
| neg_ctrl_qc | Negative control or poor quality sample |
| nelson2016_1 | Neuroendocrine Disease |
| pam50_basal | |
| portos_1 | Radiotherapy Response |
| portos_2 | Radiotherapy Response |
| rbloss_1 | RB loss status |
| smallcell_1 | Small Cell Carcinoma |
| smallcell_2 | Small Cell Carcinoma |
| smallcell_3 | Small Cell Carcinoma |
| torresroca2009_1 | Radiosensitivity |
| zhang2016_basal_1 | Basal-like |

TABLE 7
Class Four Co-Regulated Signatures

| Signature | Description |
|---|---|
| estimate2013_2_estimate | Tumor, immune, and stromal |
| | content |
| estimate2013_2_immune | Tumor, immune, and stromal |
| | content |
| estimate2013_2_stromal | Tumor, immune, and stromal |
| | content |
| hallmark_allograft_rejection | |
| hallmark_angiogenesis | |
| hallmark_complement | |
| hallmark_il2_stat5_signaling | |
| hallmark_il6_jak_stat3_signaling | |
| hallmark_inflammatory_response | |
| hallmark_interferon_alpha_response | |
| hallmark_interferon_gamma_response | |
| hallmark_kras_signaling_up | |
| immunophenoscore_1_Act.CD4 | Tumor Immunogenicity |
| immunophenoscore_1_Act.CD8 | Tumor Immunogenicity |
| immunophenoscore_1_B2M | Tumor Immunogenicity |
| immunophenoscore_1_CD27 | Tumor Immunogenicity |
| immunophenoscore_1_EC | Tumor Immunogenicity |
| immunophenoscore_1_HLA.A | Tumor Immunogenicity |
| immunophenoscore_1_HLA.B | Tumor Immunogenicity |
| immunophenoscore_1_HLA.C | Tumor Immunogenicity |
| immunophenoscore_1_HLA.DPA1 | Tumor Immunogenicity |
| immunophenoscore_1_HLA.DPB1 | Tumor Immunogenicity |
| immunophenoscore_1_HLA.E | Tumor Immunogenicity |
| immunophenoscore_1_HLA.F | Tumor Immunogenicity |
| immunophenoscore_1_IPS | Tumor Immunogenicity |
| immunophenoscore_1_IPS.raw | Tumor Immunogenicity |
| immunophenoscore_1_MHC | Tumor Immunogenicity |
| immunophenoscore_1_TAP1 | Tumor Immunogenicity |
| immunophenoscore_1_TAP2 | Tumor Immunogenicity |
| immunophenoscore_1_Tem.CD8 | Tumor Immunogenicity |

TABLE 8
References

| N | Name | V | Disease | End-point | Citation |
|---|---|---|---|---|---|
| 8 | agell2012_lm | 1 | Prostate | MET | Agell L, Hernández S, Nonell L, Lorenzo M, Puigdecanet E, de Muga S, |
| | | | Cancer | | Juanpere N, Bermudo R, Fernández P L, Lorente J A, Serrano S, Lloreta |
| | | | | | J. A 12-gene expression signature is associated with aggressive |
| | | | | | histological in prostate cancer: SEC14L1 and TCEB1 genes are |
| | | | | | potential markers of progression. Am J Pathol. 2012 November; 181(5): 1585- |
| | | | | | 94. doi: 10.1016/j.ajpath.2012.08.005. Erratum in: Am J Pathol. 2013 |
| | | | | | February; 182(2): 610. |
| 1 | angiogenesis_gene_sets | 1 | Pan | Angiogenesis | Sig0: Liberzon2015, Cell Syst 1(6): 417-425; Sig1: Masiero2013, Cancer |
| | | | Cancer | scores | Cell 24: 229-241; Sig2: Nolan2013, Developmental Cell 26: 204-219; |
| | | | | | Sig3: Brauer2013, Clin Cancer Res 19: 3681-3692; Sig4: Uhlik2016, |
| | | | | | Cancer Res 76: 2573-2586; Sig5: Kegg VEGF Signaling pathway. |
| 9 | aros | 1 | Prostate | AR activity | Faisal et al, Racial Variations in Prostate Cancer Molecular Subtypes |
| | | | Cancer | | and Androgen Receptor Signaling Reflect Anatomic Tumor |
| | | | | | Location, Eur Urol. 2015 Oct. 9 |
| 10 | AR_Related_Pathway | 1 | Prostate | ARv7 and GR | Multiple source and literature curated |
| | | | Cancer | Activity | |
| 11 | ars | 1 | Prostate | Hormone | Sharma et al., Development and validation of a prostate cancer genomic |
| | | | Cancer | Treatment | signature that predicts early ADT treatment response following radical |
| | | | | Failure | prostatectomy. Accepted for publication, Clinical Cancer Research, |
| | | | | | 2018 |
| 12 | beltran2016 | 1 | Prostate | Neuroendocrine | Beltran et al, Divergent clonal evolution of castration-resistant |
| | | | Cancer | Disease | neuroendocrine prostate cancer, Nat Med. 2016 March; 22(3): 298-305 |
| 13 | bibikova2007_lm | 1 | Prostate | MET | Marina Bibikova, Eugene Chudin, Amir Arsanjani, Lixin Zhou, Eliza |
| | | | Cancer | | Wickham Garcia, Joshua Modder, Monica Kostelec, David Barker, |
| | | | | | Tracy Downs, Jian-Bing Fan, Jessica Wang-Rodriguez. Expression |
| | | | | | signatures that correlated with Gleason score and relapse in prostate |
| | | | | | cancer. Genomics Volume 89, Issue 6, June 2007, Pages 666-672. |
| 14 | bismar2006_lm | 1 | Prostate | MET | Tarek A Bismar, Francesca Demichelis, Alberto Riva, Robert Kim, |
| | | | Cancer | | Sooryanarayana Varambally, Le He, Jeff Kutok, Jonathan C Aster, |
| | | | | | Jeffery Tang, Rainer Kuefer, Matthias D Hofer, Phillip G Febbo, Arul |
| | | | | | M Chinnaiyan, Mark A Rubin. Defining Aggressive Prostate Cancer |
| | | | | | Using a 12-Gene Model. Neoplasia. 2006 January; 8(1): 59-68. doi: |
| | | | | | 10.1593/neo.05664. |
| 15 | bismar2017 | 1 | Prostate | MET | Bismar et al., manuscript in review |
| | | | Cancer | | |
| 16 | cheville2008_lm | 1 | Prostate | MET | Cheville J C, Kames R J, Themeau T M, Kosari F, Munz J M, Tillmans L, |
| | | | Cancer | | Basal E, Rangel L J, Bergstralh E, Kovtun I V, Savci-Heijink C D, Klee |
| | | | | | E W, Vasmatzis G. Gene panel model predictive of outcome in men at |
| | | | | | high-risk of systemic progression and death from prostate cancer after |
| | | | | | radical retropubic prostatectomy. J Clin Oncol. 2008 Aug. |
| | | | | | 20; 26(24): 3930-6. doi: 10.1200/JC0.2007.15.6752. |
| 17 | cuzick2011 | 1 | Prostate | MET | Cuzick J, Swanson G P, Fisher G, Brothman A R, Bemey D M, Reid J E, |
| | | | Cancer | | Mesher D, Speights V O, Stankiewicz E, Foster C S, Moller H, Scardino |
| | | | | | P, Warren J D, Park J, Younus A, Flake D D 2nd, Wagner S, Gutin A, |
| | | | | | Lanchbury J S, Stone S; Transatlantic Prostate Group. Prognostic value |
| | | | | | of an RNA expression signature derived from cell cycle proliferation |
| | | | | | genes in patients with prostate cancer: a retrospective study. Lancet |
| | | | | | Oncol. 2011 March; 12(3): 245-55. doi: 10.1016/S1470-2045(10)70295-3. |
| 18 | cuzick2011_lm | 1 | Prostate | MET | Cuzick J, Swanson G P, Fisher G, Brothman A R, Bemey D M, Reid J E, |
| | | | Cancer | | Mesher D, Speights V O, Stankiewicz E, Foster C S, Møller H, Scardino |
| | | | | | P, Warren J D, Park J, Younus A, Flake D D 2nd, Wagner S, Gutin A, |
| | | | | | Lanchbury J S, Stone S; Transatlantic Prostate Group. Prognostic value |
| | | | | | of an RNA expression signature derived from cell cycle proliferation |
| | | | | | genes in patients with prostate cancer: a retrospective study. Lancet |
| | | | | | Oncol. 2011 March; 12(3): 245-55. doi: 10.1016/S1470-2045(10)70295-3. |
| | | | | | PMID: 21310658 |
| 19 | dasatinib_sens | 1 | Prostate | Dasatinib | Liu Y et al., manuscript in preparation |
| | | | Cancer | Sensitivity | |
| 20 | decipher | 1 | Prostate | MET | Erho N, Crisan A, Vergara I A, Mitra A P, Ghadessi M, Buerki C, |
| | | | Cancer | | Bergstralh E J, Kollmeyer T, Fink S, Haddad Z, Zimmermann B, |
| | | | | | Sierocinski T, Ballman K V, Triche T J, Black P C, Kames R J, Klee G, |
| | | | | | Davicioni E, Jenkins R B. Discovery and validation of a prostate cancer |
| | | | | | genomic classifier that predicts early metastasis following radical |
| | | | | | prostatectomy. PLoS One. 2013 Jun. 24; 8(6): e66855. doi: |
| | | | | | 10.1371/journal.pone.0066855. Print 2013. |
| 22 | decipherv2 | 2 | Prostate | MET | Spratt et al., manuscript in prepartation |
| | | | Cancer | | |
| 23 | docetaxel_sens | 1 | Prostate | Docetaxel | Liu Y et al., manuscript in preparation |
| | | | Cancer | Sensitivity | |
| 24 | ergmodel | 1 | Prostate | ERG+ | Tomlins S A, Alshalalfa M, Davicioni E, Erho N, Yousefi K, Zhao S, |
| | | | Cancer | | Haddad Z, Den R B, Dicker A P, Trock B J, DeMarzo A M, Ross A E, |
| | | | | | Schaeffer E M, Klein E A, Magi-Galluzzi C, Kames R J, Jenkins R B, |
| | | | | | Feng F Y. Characterization of 1577 primary prostate cancers reveals |
| | | | | | novel biological and clinicopathologic insights into molecular subtypes. |
| | | | | | Eur Urol. 2015 October; 68(4): 555-67. doi: 10.1016/j.eururo.2015.04.033. |
| | | | | | Epub 2015 May 8. |
| 2 | estimate2013 | 2 | Pan | Tumor, | Yoshihara K, Shahmoradgoli M, Martínez E, Vegesna R, Kim H, |
| | | | Cancer | immune, | Torres-Garcia W, Treviño V, Shen H, Laird P W, Levine D A, Carter S L, |
| | | | | and stromal | Getz G, Stemke-Hale K, Mills G B, Verhaak R. Inferring tumour purity |
| | | | | content | and stromal and immune cell admixture from expression data. Nature |
| | | | | | Communications. 2013. doi: 10.1038/ncomms3612. |
| 25 | genomic_capras | 1 | Prostate | High CAPRA-S | unpublished |
| | | | Cancer | Score | |
| 26 | genomic_gleason_grade | 1 | Prostate | Gleason | unpublished |
| | | | Cancer | Grade 4+ | |
| 27 | genomic_gleason_grade | 2 | Prostate | Gleason | unpublished |
| | | | Cancer | Grade 4+ | |
| 28 | glinsky2004_lm | 1 | Prostate | MET | Gennadi V. Glinsky, Anna B. Glinskii, Andrew J. Stephenson, Robert |
| | | | Cancer | | M. Hoffman, William L. Gerald. Gene expression profiling predicts |
| | | | | | clinical outcome of prostate cancer. Published in Volume 113, Issue 6. J |
| | | | | | Clin Invest. 2004; 113(6): 913923 doi: 10.1172/JCI20032 |
| 29 | glinsky2005_lm | 1 | Prostate | MET | Glinsky G V, Berezovska O, Glinskii A B. Microarray analysis identifies |
| | | | Cancer | | a death-from-cancer signature predicting therapy failure in patients with |
| | | | | | multiple types of cancer. J Clin Invest. 2005 June; 115(6): 1503-21. |
| 3 | hallmark_gene_sets | 1 | Pan | Biologic | Liberzon A, Birger C, Thorvaldsdóttir H, Ghandi M, Mesirov J P, |
| | | | Cancer | Gene Set | Tamayo P. The Molecular Signatures Database (MSigDB) hallmark |
| | | | | Scores | gene set collection. Cell Syst. 2015 Dec. 23; 1(6): 417-425. |
| 4 | immunophenoscore | 1 | Pan | Tumor | Charoentong P, Finotello F, Angelova M, Mayer C, Efremova M, |
| | | | Cancer | Immuno- | Rieder D, Hackl H, Trajanoski Z. Pan-cancer Immunogenomic Analyses |
| | | | | genicity | Reveal Genotype-Immunophenotype Relationships and Predictors of |
| | | | | | Response to Checkpoint Blockade. Cell Rep. 2017 Jan. 3; 18(1): 248-262. |
| | | | | | doi: 10.1016/j.celrep.2016.12.019. |
| 5 | kegg_dna_repair_gene_sets | 1 | Pan | Biologic | Kanehisa M, Goto S. KEGG: kyoto encyclopedia of genes and |
| | | | Cancer | Gene Set | genomes. Nucleic |
| | | | | Scores | Acids Res. 2000 Jan. 1; 28(1): 27-30. PubMed PMID: 10592173; PubMed |
| | | | | | Central PMCID: |
| | | | | | PMC102409. |
| 30 | klein2014_lm | 1 | Prostate | MET | Klein E A, Cooperberg M R, Magi-Galluzzi C, Simko J P, Falzarano S M, |
| | | | Cancer | | Maddala T, Chan J M, Li J, Cowan J E, Tsiatis A C, Cherbavaz D B, |
| | | | | | Pelham R J, Tenggara-Hunter I, Baehner F L, Knezevic D, Febbo P G, |
| | | | | | Shak S, Kattan M W, Lee M, Carroll P R. A 17-gene assay to predict |
| | | | | | prostate cancer aggressiveness in the context of Gleason grade |
| | | | | | heterogeneity, tumor multifocality, and biopsy undersampling. Eur Urol. |
| | | | | | 2014 September; 66(3): 550-60. doi: 10.1016/j.eururo.2014.05.004. Epub 2014 |
| | | | | | May 16. PMID: 24836057. |
| 31 | lapointe2004_lm | 1 | Prostate | MET | Lapointe J, Li C, Higgins J P, van de Rijn M, Bair E, Montgomery K, |
| | | | Cancer | | Ferrari M, Egevad L, Rayford W, Bergerheim U, Ekman P, DeMarzo |
| | | | | | A M, Tibshirani R, Botstein D, Brown P O, Brooks J D, Pollack J R. Gene |
| | | | | | expression profiling identifies clinically relevant subtypes of prostate |
| | | | | | cancer. Proc Natl Acad Sci USA. 2004 Jan. 20; 101(3): 811-6. Epub |
| | | | | | 2004 Jan. 7. |
| 32 | larkin2012_lm | 1 | Prostate | MET | S E T Larkin, S Holmes, I A Cree, T Walker, V Basketter, B Bickers, S |
| | | | Cancer | | Harris, S D Garbis, P A Townsend, C Aukim-Hastie. Identification of |
| | | | | | markers of prostate cancer progression using candidate gene expression. |
| | | | | | Br J Cancer. 2012 Jan. 3; 106(1): 157-165. Published online 2011 |
| | | | | | Nov. 10. doi: 10.1038/bjc.2011.490. |
| 33 | long2011_lm | 1 | Prostate | MET | Long Q, Johnson B A, Osunkoya A O, Lai Y H, Zhou W, Abramovitz M, |
| | | | Cancer | | Xia M, Bouzyk M B, Nam R K, Sugar L, Stanimirovic A, Williams D J, |
| | | | | | Leyland-Jones B R, Seth A K, Petros J A, Moreno C S. Protein-coding and |
| | | | | | microRNA biomarkers of recurrence of prostate cancer following |
| | | | | | radical prostatectomy. Am J Pathol. 2011 July; 179(1): 46-54. doi: |
| | | | | | 10.1016/j.ajpath.2011.03.008. Epub 2011 May 3. |
| 34 | long2014 | 1 | Prostate | BCR | Long Q, Xu J, Osunkoya A O, Sannigrahi S, Johnson B A, Zhou W, |
| | | | Cancer | | Gillespie T, Park J Y, Nam R K, Sugar L, Stanimirovic A, Seth A K, |
| | | | | | Petros J A, Moreno C S. Global transcriptome analysis of formalin-fixed |
| | | | | | prostate cancer specimens identifies biomarkers of disease recurrence. |
| | | | | | Cancer Res. 2014 Jun. 15; 74(12): 3228-37. doi: 10.1158/0008- |
| | | | | | 5472.CAN-13-2699. Epub 2014 Apr. 8. |
| 35 | lotan2016 | 1 | Prostate | Neuro- | Tsai H K, Lehrer J, Alshalalfa M, Erho N, Davicioni E, Lotan T L. Gene |
| | | | Cancer | endocrine | expression signatures of neuroendocrine prostate cancer and primary |
| | | | | | small cell prostatic carcinoma. BMC Cancer. 2017 Nov. 13; 17(1): 759. |
| | | | | | doi: 10.1186/sl2885-017-3729-z. |
| 36 | nakagawa2008_lm | 1 | Prostate | MET | Tohru Nakagawa, Thomas M. Kollmeyer, Bruce W. Morlan, S. Keith |
| | | | Cancer | | Anderson, Eric J. Bergstralh, Brian J. Davis, Yan W. Asmann, George |
| | | | | | G. Klee, Karla V. Ballman, Robert B. Jenkins. A Tissue Biomarker |
| | | | | | Panel Predicting Systemic Progression after PSA Recurrence Post- |
| | | | | | Definitive Prostate Cancer Therapy. PLoS ONE. 2008; 3(5): e2318. |
| | | | | | Published online 2008 May 28. doi: 10.1371/journal.pone.0002318. |
| 37 | neg_ctrl_qc | 1 | Prostate | Negative | unpublished |
| | | | Cancer | control or | |
| | | | | poor quality | |
| | | | | sample | |
| 38 | nelson2016 | 1 | Prostate | Neuro- | Kumar et al, Substantial interindividual and limited intraindividual |
| | | | Cancer | endocrine | genomic diversity among tumors from men with metastatic prostate |
| | | | | Disease | cancer, Nature Medicine 22, 369-378 |
| 39 | nelson_2016_AR | 1 | Prostate | AR Activity | Kumar A, Coleman I, Morrissey C, Zhang X, True L D, Gulati R, |
| | | | Cancer | | Etzioni R, Bolouri H, Montgomery B, White T, Lucas J M, Brown L G, |
| | | | | | Dumpit R F, DeSarkar N, Higano C, Yu E Y, Coleman R, Schultz N, |
| | | | | | Fang M, Lange P H, Shendure J, Vessella R L, Nelson P S. Substantial |
| | | | | | interindividual and limited intraindividual genomic diversity among |
| | | | | | tumors from men with metastatic prostate cancer. Nat Med. 2016 |
| | | | | | April; 22(4): 369-78. doi: 10.1038/nm.4053. Epub 2016 Feb. 29. PubMed |
| | | | | | PMID: 26928463. |
| 40 | non_organ_confined | 1 | Prostate | Non-organ | unpublished |
| | | | Cancer | confined | |
| | | | | prostate | |
| | | | | cancer at | |
| | | | | RP | |
| 41 | normaltumor | 1 | Prostate | tumor | unpublished |
| | | | Cancer | | |
| 42 | pam50 | 1 | Prostate | Subtypes | Parker J S, Mullins M, Cheang M C, Leung S, Voduc D, Vickery T, |
| | | | Cancer | | Davies S, Fauron C, He X, Hu Z, Quackenbush J F, Stijleman I J, Palazzo |
| | | | | | J, Marron J S, Nobel A B, Mardis E, Nielsen T O, Ellis M J, Perou C M, |
| | | | | | Bernard P S. Supervised risk predictor of breast cancer based on intrinsic |
| | | | | | subtypes. J Clin Oncol. 2009 Mar. 10; 27(8): 1160-7. doi: |
| | | | | | 10.1200/JCO.2008.18.1370. |
| 43 | pca_vs_mibc | 1 | Prostate | Prostate | unpublished |
| | | | Cancer | Cancer Vs | |
| | | | | Bladder | |
| | | | | Cancer | |
| 44 | penney2011 | 1 | Prostate | PCSM | Penney K L1, Sinnott J A, Fall K, Pawitan Y, Hoshida Y, Kraft P, Stark |
| | | | Cancer | | J R, Fiorentino M, Perner S, Finn S, Calza S, Flavin R, Freedman M L, |
| | | | | | Setlur S, Sesso H D, Andersson S O, Martin N, Kantoff P W, Johansson |
| | | | | | J E, Adami H O, Rubin M A, Loda M, Golub T R, Andrén O, Stampfer |
| | | | | | M J, Mucci L A. |
| 45 | penney2011_lm | 1 | Prostate | MET | Kathryn L. Penney, Jennifer A. Sinnott, Katja Fall, Yudi Pawitan, Yujin |
| | | | Cancer | | Hoshida, Peter Kraft, Jennifer R. Stark, Michelangelo Fiorentino, Sven |
| | | | | | Perner, Stephen Finn, Stefano Calza, Richard Flavin, Matthew L. |
| | | | | | Freedman, Sunita Setlur, Howard D. Sesso, Swen-Olof Andersson, Neil |
| | | | | | Martin, Philip W. Kantoff, Jan-Erik Johansson, Hans-Olov Adami, |
| | | | | | Mark A. Rubin, Massimo Loda, Todd R. Golub, Ove Andren, Meir J. |
| | | | | | Stampfer, Lorelei A. Mucci. mRNA Expression Signature of Gleason |
| | | | | | Grade Predicts Lethal Prostate Cancer. J Clin Oncol. 2011 Jun. 10; |
| | | | | | 29(17): 2391-2396. Published online 2011 May 2. doi: |
| | | | | | 10.1200/JCO.2010.32.6421. |
| 46 | portos | 1 | Prostate | Radiotherapy | |
| | | | Cancer | Response | |
| 47 | portos | 2 | Prostate | Radiotherapy | Zhao S G, Chang S L, Spratt D E, Erho N, Yu M, Ashab H A, Alshalalfa |
| | | | Cancer | Sensitivity | M, Speers C, Tomlins S A, Davicioni E, Dicker A P, Carroll P R, |
| | | | | | Cooperberg M R, Freedland S J, Karnes R J, Ross A E, Schaeffer E M, Den |
| | | | | | R B, Nguyen P L, Feng F Y. Development and validation of a 24-gene |
| | | | | | predictor of response to postoperative radiotherapy in prostate cancer: a |
| | | | | | matched, retrospective analysis. Lancet Oncol. 2016 November; 17(11): 1612- |
| | | | | | 1620. doi: 10.1016/S1470-2045(16)30491-0. |
| 6 | race | 1 | Pan | Race | unpublished |
| | | | Cancer | | |
| 48 | ragnum2015 | 1 | Prostate | Pimonidazole | Ragnum H B, Vlatkovic L, Lie A K, Axcrona K, Julin C H, Frikstad K M, |
| | | | Cancer | | Hole K H, Seierstad T, Lyng H. The tumour hypoxia marker |
| | | | | | pimonidazole reflects a transcriptional programme associated with |
| | | | | | aggressive prostate cancer. Br J Cancer. 2015 Jan. 20; 112(2): 382-90. |
| | | | | | doi: 10.1038/bjc.2014.604. Epub 2014 Dec. 2. |
| 49 | Ramaswamy2003_lm | 1 | Prostate | MET | Ramaswamy S, Ross K N, Lander E S, Golub T R. A molecular signature |
| | | | Cancer | | of metastasis in primary solid tumors. Nat Genet. 2003 January; 33(1): 49-54. |
| | | | | | Epub 2002 Dec. 9. |
| 7 | rbloss | 1 | Pan | RB loss | unpublished |
| | | | Cancer | status | |
| 50 | saal2007_lm | 1 | Prostate | MET | Saal L H, Johansson P, Holm K, Gruvberger-Saal S K, She Q B, Maurer |
| | | | Cancer | | M, Koujak S, Fernando A A, Malmström P, Memeo L, Isola J, Bendahl |
| | | | | | P O, Rosen N, Hibshoosh H, Ringńer M, Borg A, Parsons R. Poor |
| | | | | | prognosis in carcinoma is associated with a gene expression signature of |
| | | | | | aberrant PTEN tumor suppressor pathway activity. Proc Natl Acad Sci |
| | | | | | USA. 2007 May 1; 104(18): 7564-9. Epub 2007 Apr. 23. |
| 51 | saal2007_pten | 2 | Prostate | PTEN Loss | Saal L H, Johansson P, Holm K, Gruvberger-Saal S K, She Q B, Maurer |
| | | | Cancer | | M, Koujak S, Fernando A A, Malmström P, Memeo L, Isola J, Bendahl |
| | | | | | P O, Rosen N, Hibshoosh H, Ringńer M, Borg A, Parsons R. Poor |
| | | | | | prognosis in carcinoma is associated with a gene expression signature of |
| | | | | | aberrant PTEN tumor suppressor pathway activity. Proc Natl Acad Sci |
| | | | | | USA. 2007 May 1; 104(18): 7564-9. Epub 2007 Apr. 23. |
| 52 | sdms | 1 | Prostate | MET | Mo F, Lin D, Takhar M, Ramnarine V R, Dong X, Bell R H, Volik S V, |
| | | | Cancer | | Wang K, Xue H, Wang Y, Haegert A, Anderson S, Brahmbhatt S, Erho |
| | | | | | N, Wang X, Gout P W, Morris J, Karnes R J, Den R B, Klein E A, |
| | | | | | Schaeffer E M, Ross A, Ren S, Sahinalp S C, Li Y, Xu X, Wang J, Wang |
| | | | | | J, Gleave M E, Davicioni E, Sun Y, Wang Y, Collins C C, Stromal Gene |
| | | | | | Expression is Predictive for Metastatic Primary Prostate Cancer. Eur |
| | | | | | Urol. 2017 March; S0302-2838(17)30166-5. doi: |
| | | | | | 10.1016/j.eururo.2017.02.038. |
| 53 | singh2002_lm | 1 | Prostate | MET | Singh D, Febbo P G, Ross K, Jackson D G, Manola J, Ladd C, Tamayo P, |
| | | | Cancer | | Renshaw A A, DAmico A V, Richie J P, Lander E S, Loda M, Kantoff |
| | | | | | P W, Golub T R, Sellers W R. Gene expression correlates of clinical |
| | | | | | prostate cancer behavior. Cancer Cell. 2002 March; 1(2): 203-9. |
| 54 | smallcell | 1 | Prostate | Small Cell | unpublished |
| | | | Cancer | Carcinoma | |
| 55 | smallcell | 2 | Prostate | Small Cell | unpublished |
| | | | Cancer | Carcinoma | |
| 56 | smallcell | 3 | Prostate | Small Cell | Alshalafa et al., manuscript in preparation |
| | | | Cancer | Carcinoma | |
| 57 | staging_epe | 1 | Prostate | EPE | Hall W et al., manuscript in preparation |
| | | | Cancer | | |
| 58 | staging_lni | 1 | Prostate | LNI | Spratt et al., manuscript in prepartation |
| | | | Cancer | | |
| 59 | staging_svi | 1 | Prostate | SVI | Hall W et al., manuscript in preparation |
| | | | Cancer | | |
| 60 | stephenson2005_lm | 1 | Prostate | MET | Stephenson A J, Smith A, Kattan M W, Satagopan J, Reuter V E, |
| | | | Cancer | | Scardino P T, Gerald W L. Integration of gene expression profiling and |
| | | | | | clinical variables to predict prostate carcinoma recurrence after radical |
| | | | | | prostatectomy. Cancer. 2005 Jul. 15; 104(2): 290-8. |
| 61 | talantov2010_lm | 1 | Prostate | MET | Talantov D, Jatkoe T A, Böhm M, Zhang Y, Ferguson A M, Stricker P D, |
| | | | Cancer | | Kattan M W, Sutherland R L, Kench J G, Wang Y, Henshall S M. Gene |
| | | | | | based prediction of clinically localized prostate cancer progression after |
| | | | | | radical prostatectomy. J Urol. 2010 October; 184(4): 1521-8. doi: |
| | | | | | 10.1016/j.juro.2010.05.084. Epub 2010 Aug. 17. |
| 62 | torresroca2009 | 1 | Prostate | Radio- | Eschrich S A, Pramana J, Zhang H, Zhao H, Boulware D, Lee J H, |
| | | | Cancer | sensitivity | Bloom G, Rocha-Lima C, Kelley S, Calvin D P, Yeatman T J, Begg A C, |
| | | | | | Torres-Roca J F. A gene expression model of intrinsic tumor |
| | | | | | radiosensitivity: prediction of response and prognosis after |
| | | | | | chemoradiation. Int J Radiat Oncol Biol Phys. 2009 Oct. 1; 75(2): 489-96. |
| | | | | | doi: 10.1016/j.ijrobp.2009.06.014. |
| 63 | varambally2005_lm | 1 | Prostate | MET | Varambally S, Yu J, Laxman B, Rhodes D R, Mehra R, Tomlins S A, |
| | | | Cancer | | Shah R B, Chandran U, Monzon F A, Becich M J, Wei J T, Pienta K J, |
| | | | | | Ghosh D, Rubin M A, Chinnaiyan A M. Integrative genomic and |
| | | | | | proteomic analysis of prostate cancer reveals signatures of metastatic |
| | | | | | progression. Cancer Cell. 2005 November; 8(5): 393-406. |
| 64 | wu2013_lm | 1 | Prostate | MET | Chin-Lee Wu, Brock E. Schroeder, Xiao-Jun Ma, Christopher J. Cutie, |
| | | | Cancer | | Shulin Wu, Ranelle Salunga, Yi Zhang, Michael W. Kattan, Catherine |
| | | | | | A. Schnabel, Mark G. Erlander, W. Scott McDougal. Development and |
| | | | | | validation of a 32-gene prognostic index for prostate cancer progression. |
| | | | | | Proc Natl Acad Sci USA. 2013 Apr. 9; 110(15): 6121-6126. |
| | | | | | Published online 2013 Mar. 26. doi: 10.1073/pnas.1215870110. |
| 65 | yu2007_lm | 1 | Prostate | MET | Yu J, Yu J, Rhodes D R, Tomlins S A, Cao X, Chen G, Mehra R, Wang |
| | | | Cancer | | X, Ghosh D, Shah R B, Varambally S, Pienta K J, Chinnaiyan A M. A |
| | | | | | polycomb repression signature in metastatic prostate cancer predicts |
| | | | | | cancer outcome. Cancer Res. 2007 Nov. 15; 67(22): 10657-63. |
| 66 | zhang2016_basal | 1 | Prostate | Basal-like | Zhang D, Park D, Zhong Y, Lu Y, Rycaj K, Gong S, Chen X, Liu X, |
| | | | Cancer | | Chao H P, Whitney P, Calhoun-Davis T, Takata Y, Shen J, Iyer V R, |
| | | | | | Tang D G. Stem cell and neurogenic gene-expression profiles link |
| | | | | | prostate basal cells to aggressive prostate cancer. Nat Commun. 2016 |
| | | | | | Feb. 29; 7: 10798. doi: 10.1038/ncomms10798. PubMedPMID: |
| | | | | | 26924072; PubMed Central PMCID: PMC4773505. |

V refers to version.

What is claimed is:

1. A method of providing improved treatment benefit of non-metastatic castration resistant prostate cancer (nmCRPC) in a human male using apalutamide (APA) and an androgen deprivation therapy (ADT) (APA+ADT), said method comprising
administering a therapeutically effective amount of APA+ADT to the human male if a biological sample obtained from the human male is determined to have:
a) a luminal-like molecular subtype of prostate cancer;
b) a genomic classifier score of greater than about 0.6;
c) an increased expression of at least one signature of Class One co-regulated signatures;
d) an increased expression of at least one signature of Class Two co-regulated signatures;
e) a decreased expression of at least one signature of Class Three co-regulated signatures;
f) an increased expression of at least one signature of Class Four co-regulated signatures;
or a combination thereof.

2. A method of treating non-metastatic castration resistant prostate cancer (nmCRPC) in a human male, said method comprising
administering a therapeutically effective amount of apalutamide (APA) and a therapeutically effective amount of an androgen deprivation therapy (ADT) (APA+ADT) to the human male if a biological sample originated from the human male is determined to have:
a) a luminal-like molecular subtype of prostate cancer;
b) a genomic classifier score of greater than about 0.6;
c) an increased expression of at least one signature of Class One co-regulated signatures;
d) an increased expression of at least one signature of Class Two co-regulated signatures;
e) a decreased expression of at least one signature of Class Three co-regulated signatures;
f) an increased expression of at least one signature of Class Four co-regulated signatures;
or a combination thereof.

3. A method of predicting a human male having a non-metastatic castration resistant prostate cancer (nmCRPC) to have an improved benefit from administration of a therapeutically effective amount of apalutamide (APA) and a therapeutically effective amount of an androgen deprivation therapy (ADT) (APA+ADT), said method comprising
a) determining if a biological sample obtained from the human male has:
i) a luminal-like molecular subtype of prostate cancer;
ii) a genomic classifier score of greater than about 0.6;
iii) an increased expression level of at least one signature of the Class One, Class Two, and/or Class Four co-regulated signatures;
iv) a decreased expression level of at least one signature of the Class Three co-regulated signatures;
or a combination thereof, and
b) predicting that the human male to have an improved benefit from administration of the therapeutically effective amount of APA+ADT based on:
i) a luminal-like molecular subtype of prostate cancer;
ii) a genomic classifier score of greater than about 0.6;
iii) an increased expression level of at least one signature of the Class One, Class Two, and/or Class Four co-regulated signatures;
iv) a decreased expression level of at least one signature of the Class Three co-regulated signatures;
or a combination thereof.

4. A method of improving response to treating non-metastatic castration resistant prostate cancer (nmCRPC) in a human male using a combined administration of a therapeutically effective amount of apalutamide (APA) and a therapeutically effective amount of an androgen deprivation therapy (ADT) (APA+ADT), the method comprising
a) determining if a biological sample obtained from the human male has:
i) a luminal-like molecular subtype of prostate cancer;
ii) a genomic classifier score of greater than about 0.6;
iii) an increased expression level of at least one signature of the Class One, Class Two, and/or Class Four co-regulated signatures;
iv) a decreased expression level of at least one signature of the Class Three co-regulated signatures;
or a combination thereof, and
b) improving response to combined administration of the therapeutically effective amount of APA+ADT, based on:
i) a luminal-like molecular subtype of prostate cancer;
ii) a genomic classifier score of greater than about 0.6;
iii) an increased expression level of at least one signature of the Class One, Class Two, and/or Class Four co-regulated signatures;
iv) a decreased expression level of at least one signature of the Class Three co-regulated signatures;
or a combination thereof.

5. The method of any one of claims 1-4, wherein the human male has undergone a prostatectomy.

6. The method of any one of claims 1-5, wherein the biological sample is a prostate biopsy sample or a surgical tumor sample.

7. The method of any one of claims 1-5, wherein the biological sample is a primary prostate tumor sample.

8. The method of any one of claims 1-7, wherein metastasis-free survival (MFS) of combined administration of APA+ADT is improved by at least about 6 months relative to sole administration of ADT alone.

9. The method of any one of claims 1-8, wherein second progression-free survival (PFS2) of combined administration of APA+ADT is improved by at least about 6 months relative to sole administration of ADT alone.

10. The method of any one of claims 1-9, wherein the administering is by oral administration.

11. The method of any one of claims 1-10, wherein the biological sample is determined to have a luminal-like molecular subtype of prostate cancer.

12. The method of any one of claims 1-11, wherein the biological sample is determined to have a genomic classifier score of greater than 0.6.

13. The method of claim 12, wherein the genomic classifier is a 22-marker genomic classifier comprising, consisting of and/or consisting essentially of markers selected from the group consisting of LASP1, IQGAP3, NFIB, S1PR4, THBS2, ANO7, PCDH7, MYBPC1, EPPK1, TSBP, PBX1, NUSAP1, ZWILCH, UBE2C, CAMKC2N1, RABGAP1, PCAT-32, GYATL1P4/PCAT-80, TNFRSF19 and combinations thereof.

14. The method of claim 12 or 13, wherein the human male is determined to have a high risk of metastasis based on the genomic classifier score.

15. The method of any one of claims 1-14, wherein the biological sample is determined to have an increased expression of at least one signature of the Class One co-regulated signatures.

16. The method of claim 15, wherein the at least one signature of the Class One co-regulated signatures is selected from the group consisting of: agell2012_1, bibikova2007_1, bismar2006_1, bismar2017_1, cheville2008_1, cuzick2011_1, cuzick2011_lm_1, decipher_1, decipherv2_2, genomic_capras_1, genomic_gleason_grade_1, genomic_gleason_grade_2, glinsky2005_1, hallmark_mtorc1_signaling, hallmark_myc_targets_v1, hallmark_myc_targets_v2, klein2014_1, lapointe2004_1, larkin2012_1, long2014_1, nakagawa2008_1, non_organ_confined_1, normaltumor_1, pam50_luminalB, penney2011_1, penney2011_lm_1, ramaswamy2003_1, saa12007_1, saal2007_pten, sdms_1, singh2002_1, staging_epe_1, staging_lni_1, staging_svi_1, stephenson2005_1, talantov2010_1, varambally2005_1, wu2013_1, yu2007_1, and combinations thereof.

17. The method of claim 16, wherein the at least one signature of the Class One co-regulated signatures comprises genomic_gleason_grade_2.

18. The method of any one of claims 1-17, wherein the biological sample is determined to have an increased expression of at least one signature of the Class Two co-regulated signatures.

19. The method of claim 18, wherein the at least one signature of the Class Two co-regulated signatures is selected from the group consisting of: ar_related_pathway_ARv7, ar_related_pathway_glucocorticoid_receptor, aros_1, docetaxel_sens_1, ergmodel_1, glinsky2004_1, hallmark_adipogenesis, hallmark_androgen_response, hallmark_angiogenesis_Brauer2013, hallmark_angiogenesis_KeggVEGF, hallmark_angiogenesis_Liberzon2015, hallmark_angiogenesis_Masiero2013, hallmark_angiogenesis_Nolan2013, hallmark_angiogenesis_Uhlik2016, hallmark_apical_surface, hallmark_bile_acid_metabolism, hallmark_cholesterol_homeostasis, hallmark_dna_repair, hallmark_e2f_targets, hallmark_fatty_acid_metabolism, hallmark_g2m_checkpoint, hallmark_glycolysis, hallmark_hedgehog_signaling, hallmark_heme_metabolism, hallmark_mitotic_spindle, hallmark_notch_signaling, hallmark_oxidative_phosphorylation, hallmark_peroxisome, hallmark_pi3k_akt_mtor_signaling, hallmark_protein_secretion, hallmark_spermatogenesis, hallmark_unfolded_protein_response, hallmark_uv_response_dn, hallmark_xenobiotic_metabolism, immunophenoscore_1_CP, immunophenoscore_1_CTLA.4, immunophenoscore_1_IDOL immunophenoscore_1_LAG3, immunophenoscore_1_PD.1, immunophenoscore_1_PD.L2, immunophenoscore_1_Tem.CD4, immunophenoscore_1_TIGIT, kegg_mismatch_repair, kegg_non_homologous_end_joining, kegg_nucleotide_excision_repair, long2011_1, nelson_2016_AR_1, pam50_luminalA, pca_vs_mibc_1, race_1, ragnum2015_1, and combinations thereof.

20. The method of claim 19, wherein the at least one signature of the Class Two co-regulated signatures comprises hallmark_cholesterol_homeostasis.

21. The method of any one of claims 1-20, wherein the biological sample is determined to have a decreased expression of at least one signature of the Class Three co-regulated signatures.

22. The method of claim 21, wherein the at least one signature of the Class Three co-regulated signatures is selected from the group consisting of: ars_1, beltran2016_1, dasatinib_sens_1, estimate2013_2_purity, hallmark_apical_junction, hallmark_apoptosis, hallmark_coagulation, hallmark_epithelial_mesenchymal_transition, hallmark_estrogen_response_early, hallmark_estrogen_response_late, hallmark_hypoxia, hallmark_kras_signaling_dn, hallmark_myogenesis, hallmark_p53_pathway, hallmark_pancreas_beta_cells, hallmark_reactive_oxigen_species_pathway, hallmark_tgf_beta_signaling, hallmark_tnfa_signaling_via_nfkb, hallmark_uv_response_up, hallmark_wnt_beta_catenin_signaling, immunophenoscore_1_ICOS, immunophenoscore_1_MDSC, immunophenoscore_1_PD.L1, immunophenoscore_1_SC, immunophenoscore_1_TIM3, immunophenoscore_1_Treg, kegg_base_excision_repair, kegg_homologous_recombination, lotan2016_1, neg_ctrl_qc, nelson2016_1, pam50_basal, portos_1, portos_2, rbloss_1, smallcell_1, smallcell_2, smallcell_3, torresroca2009_1, zhang2016_basal_1, and combinations thereof.

23. The method of claim 22, wherein the at least one signature of the Class Three co-regulated signatures comprises beltran2016_1.

24. The method of any one of claims 1-23, wherein the biological sample is determined to have increased expression of at least one signature of the Class Four co-regulated signatures.

25. The method of claim 24, wherein the at least one signature of the Class Four co-regulated signatures is selected from the group consisting of: estimate2013_2_estimate, estimate2013_2_immune, estimate2013_2_stromal, hallmark allograft rejection, hallmark_angiogenesis, hallmark_complement, hallmark_IL2_JAK_STAT5_signaling, hallmark_IL6_JAK_STAT3_signaling, hallmark_inflammatory_response, hallmark_interferon_alpha_response, hallmark_interferon_gamma_response, hallmark_kras_signaling_up, immunophenoscore_1_Act.CD4, immunophenoscore_1_Act.CD8, immunophenoscore_1_B2M, immunophenoscore_1_CD27, immunophenoscore_1_EC, immunophenoscore_1_HLA.A, immunophenoscore_1_HLA.B, immunophenoscore_1_HLA.C, immunophenoscore1_HLA.DPA1, immunophenoscore_1_HLA.DPB1, immunophenoscore_1_HLA.E, immunophenoscore_1_HLA.F, immunophenoscore_1_IPS, immunophenoscore_1_IP S.raw, immunophenoscore_1_MHC, immunophenoscore_1_TAP1, immunophenoscore_1_TAP2, immunophenoscore_1_Tem.CD8, and combinations thereof.

26. The method of claim 25, wherein the at least one signature of the Class Four co-regulated signatures comprises hallmark_IL2_JAK_STAT5_signaling.
